(19) United States
(12) Patent Application Publication
(10) Pub. No.: US 2010/0104540 A1
(43) Pub. Date: Apr. 29, 2010

(54) METHODS AND COMPOSITIONS FOR TREATMENT OF FIBROSIS

(75) Inventors: Costica Aloman, North Attleboro, MA (US); Scott Friedman, Scarsdale, NY (US); Miriam Merad, New York, NY (US)

Correspondence Address: DARBY & DARBY P.C., P.O. BOX 770, Church Street Station, New York, NY, 10008-0770, US

(73) Assignee: Mount Sinai School of Medicine of New York University, New York, NY (US)

(21) Appl. No.: 12/570,871

(22) Filed: Sep. 30, 2009

Related U.S. Application Data
(60) Provisional application No. 61/108,462, filed on Oct. 24, 2008.

(51) Int. Cl. A61K 35/12; A61K 31/7088; A61K 38/19; A61K 38/18

(52) U.S. Cl. 424/93.7; 514/44 R; 514/12

(57) ABSTRACT

Treatment methods and compositions for the treatment of fibrosis are provided. In some embodiments, these methods include augmentation of dendritic cells for treatment of fibrosis. In some embodiments, fms-like tyrosine kinase 3 ligand (Flt3L) is used for the treatment of fibrosis and/or the augmentation of dendritic cells. In certain embodiments, the invention relates to methods for the treatment of fibrosis using Flt3L-expanded dendritic cells. In certain embodiments, the fibrosis is hepatic or pulmonary fibrosis.

### CROSS REFERENCE TO RELATED APPLICATIONS

The present application claims priority under 35 U.S.C. §119 to U.S. Provisional Application Ser. No. 61/108,462, filed Oct. 24, 2008, which is hereby incorporated by reference in its entirety.

### GOVERNMENT SPONSORED RESEARCH OR DEVELOPMENT

This invention was made in part in the course of research sponsored by the American Association for the Study of Liver Diseases. This agency may have certain rights in this invention.

### FIELD OF THE INVENTION

The present invention relates to treatment methods and compositions for the treatment of fibrosis. In certain embodiments, the invention relates to methods of augmenting dendritic cells for the treatment of fibrosis. The invention also relates to the use of fms-like tyrosine kinase 3 ligand (Flt3L) for the treatment of fibrosis and the augmentation of dendritic cells. In certain embodiments, the invention relates to methods for the treatment of fibrosis using Flt3L-expanded dendritic cells. In certain embodiments, the fibrosis is hepatic or pulmonary fibrosis.

### BACKGROUND OF THE INVENTION

Hepatic fibrosis, or scarring of the liver, is a wound-healing response that engages a range of cell types and mediators to encapsulate injury. Sustained signals associated with chronic liver disease caused by infection, drugs, metabolic disorders, or immune attack are required for significant fibrosis to accumulate, although even acute injury will activate mechanisms of fibrogenesis. Cirrhosis is the most advanced stage of fibrosis, and is associated with greater scarring than fibrosis alone, and with distortion of the liver parenchyma associated with septae and nodule formation, altered blood flow, and risk of liver failure. Evidence that fibrosis and even cirrhosis are reversible has intensified interest in understanding the regulation of matrix degradation and fibrosis resolution, in hopes that therapies might exploit those endogenous pathways that reverse disease [Friedman (2008) Gastroenterology; 134: 1655-1669].

A hallmark of fibrosis is the change in the composition of the extracellular matrix (ECM) within the subendothelial space of Disse. Over time, the subendothelial matrix composition changes from one comprised of type IV collagen, heparan sulfate proteoglycan, and laminin (the classic constituents of the basal lamina) to one rich in fibril-forming collagens, particularly types I and III. The progressive changes in ECM composition as fibrosis accumulates instigate several positive feedback pathways that further amplify fibrosis, including changes in the expression of integrins, activation of cellular matrix metalloproteases and enhanced density of ECM [Friedman (2008) Gastroenterology; 134: 1655-1669].

The elucidation of novel pathways of immune regulation in liver and their impact on fibrogenesis is critical to uncovering the basis of hepatic fibrosis. Even though the most prevalent liver diseases (e.g., hepatitis B virus (HBV) and hepatitis C virus (HCV)) are characterized by inflammatory infiltration and immune activation, current understanding in the art of how the immune system modulates hepatic fibrosis is limited.

Studies over the past two decades establish the hepatic stellate cell, a resident perisinusoidal cell in normal liver, as the major source of extracellular matrix following their activation into contractile myofibroblast [Albanis, E. and S. L. Friedman, (2001) Clin Liver Dis. 5:315-34; Friedman, S. L., et al., (1985) Proc Natl Acad Sci USA 82:8681-5; Bataller, R. and D. A. Brenner, (2005) J Clin Invest, 115:209-18]. As fibrosis advances, the collagenous bands typical of end-stage cirrhosis contain large numbers of activated stellate cells [Friedman (2008) Gastroenterology; 134: 1655-1669]. These cells progressively impede portal blood flow by both constricting individual sinusoids and by contracting the cirrhotic liver, mediated by pathways that allow interaction with the ECM. At the same time, stellate cell density and coverage of the sinusoidal lumen increases.

Initial links between immune function in liver and fibrogenic cells have recently been uncovered, including reports that hepatic stellate cells are professional antigen-presenting cells with the ability to take up, process, cross-present and initiate an immune response [Vinas, O., et al., (2003) Hepatology 38:919-29; Winau, F., et al., (2007) Immunity 26:117-29; Mehal, W. Z. and Friedman S. L. (2007) Liver Immunol. 2:99-109]. More recently, cells of the immune system have also been implicated in fibrosis pathogenesis, including, in particular, dendritic cells (DCs), natural killer (NK) cells and cytolytic T cells. Among these, DCs appear to play a ‘master role’ by controlling activity of NK and CD8⁺ T cells, as well as by secreting molecules that regulate matrix degradation.

DCs are professional antigen-presenting cells capable of capturing and processing antigens into immunogenic peptides that are subsequently presented along with products of the major histocompatibility complex (MHC) to T cells, thereby initiating an immune response. Upon antigen recognition and processing, DCs undergo a maturation process leading to increased expression of co-stimulatory molecules (CD80, CD86, CD40) and ability to stimulate T helper cells to initiate an immune response [Steinman, R. M. and H. Hemmi, (2006) Curr Top Microbiol Immunol 311:17-58; Steinman, R. M., (2006) Novartis Found Symp 279:101-9]. In addition to their role in initiating the immune response, DCs can also stimulate the development of tolerance. To accomplish these myriad functions, DCs secrete a range of cytokines that modulate the magnitude (IL-6, IL-1β) and the type of adaptive immune response (IL-12, INFγ, TNFα predispose to Tₕ1, and IL-10, IL-4 to Tₕ2 responses).

It is now known that a major pathway of DC maturation is through ligation of certain receptors expressed on DCs known as Toll-like receptors (TLRs). TLRs recognize conserved molecular patterns expressed on pathogens and thereby facilitate recognition of infection by the innate immune system. A number of cells, including DCs and hepatic stellate cells, have been shown to express TLR4, the receptor for bacterial lipopolysaccharide (LPS). TLR4 may play an important role in disease processes such as fibrosis, since endogenous ligands present in fibrosis, such as high-mobility group box 1, biglycan, and heparan sulfate, also may trigger TLR4 signaling [Friedman (2008) Gastroenterology; 134: 1655-1669], leading to DC activation and maturation.

After undergoing maturation, DCs activate natural killer (NK) cells under the influence of IL-15, which is also secreted by stellate cells [Winau, F., et al., (2007) Immunity 26:117-29; Lucas, M., et al., (2007) Immunity 26:503-17]. Concurrently, DC number and function are modulated by NK cells [Guan, H., et al., (2007) J Immunol 179:590-6; Pan, P. Y., et al., (2004) J Immunol 172:4779-89; He, Y., et al., (2000) Hum Gene Ther 11:547-54]. Previous studies indicate that NK cells in turn have an antifibrotic effect by promoting apoptosis of stellate cells via a TRAIL-mediated mechanism [Melhem, A., et al., (2006) J Hepatol 45:60-71; Gao, B. et al. (2008) J Hepatol 47:729-736; Jeong, W. I. et al. (2008) Gastroenterology 134:248-258]. This effect is restricted to activated but not quiescent stellate cells, since only activated stellate cells express the NK cell activating-receptor NKG2D [Radaeva S. et al. (2006) Gastroenterology 130:435-452]. Furthermore, IL-15, secreted by stellate cells [Winau, F., et al., (2007) Immunity 26:117-29], stimulates cytolytic effector function and facilitates the survival of CD8⁺ memory T cells, which, in contrast to NK cells, can promote fibrosis [Friedman (2008) Gastroenterology 134: 1655-1669]. As noted, however, CD8+ T cell responses are under the control of DCs and NK cells.

A major determinant of progressive fibrosis is the failure to degrade the increased fibril-forming, or interstitial, scar matrix, a process for which matrix metalloproteinases (MMPs) are critical. Stellate cells are thought to be the main source of MMP-2, MMP-9 and MMP-3, as well as the interstitial collagenase, MMP-13 (the rodent equivalent of human MMP-1). MMP-1 is the main protease that can degrade type I collagen, the principal collagen in fibrotic liver, and thus may play an important role in fibrosis resolution. Importantly, DCs also have the potential to play a role in modulating fibrosis, since it has been shown that DCs express, produce and secrete functionally active MMPs, including MMP-1, MMP-2, MMP-3, and MMP-9, as well as MMP inhibitors, such as the tissue inhibitors of metalloproteinases (TIMP) TIMP-1 and TIMP-2 [13] Kouwenhoven, M., et al., (2002) J Neuroimmunol 126:161-71. Recently, it was also shown that MMP-9 secreted by DCs has an important role for DCs migration from the periphery to lymph nodes [Yen, J. H., T. Khayrullina, and D. Ganea, (2008) Blood 111:260-70]. Presently, it remains unknown whether hepatic DCs have similar features, however.

It has been difficult to treat hepatic fibrosis and related diseases in humans, in part because it is difficult to acquire sufficient numbers of cells, such as immune cells, from a patient in order to perform studies that would help determine new methods of treatment. One cannot simply transfer cells from another source into a patient, (1) because it is difficult obtaining adequate supplies of such cells, and (2), problems associated with immune rejection of transferred, heterologous cells, as well potential transmission of infections prevent such approaches. Thus, at present, there is a need for methods for treating hepatic fibrosis and related diseases in humans.

### SUMMARY OF THE INVENTION

In certain embodiments, the present invention provides a method for treating fibrosis which involves administering to a mammal or a patient in need of such treatment an effective amount for treating fibrosis of Flt3L. In yet another embodiment, the invention provides a method for treating fibrosis which involves administering to a patient in need of such treatment a plasmid carrying the nucleic acid sequence of Flt3L, wherein the plasmid is administered in an amount that is effective for yielding expression of Flt3L in said patient; and wherein the Flt3L is expressed in an amount that is effective for the treatment of fibrosis.

In a certain other embodiment, the present invention provides a method for treating fibrosis which involves administering to a patient in need of such treatment an effective amount for treating fibrosis of a cell expressing and secreting Flt3L, wherein the Flt3L is secreted in an amount effective for the treatment of fibrosis. In still another embodiment, the present invention provides a method for treating fibrosis which involves administering to a mammal or patient in need of such treatment an effective amount for treating fibrosis of a dendritic cell or a dendritic cell expanded from CD34⁺ progenitor cells treated with Flt3L. In certain other embodiments, the dendritic cell may be expanded from bone marrow cells using any suitable cytokine useful for inducing differentiation of bone marrow cells into dendritic cells.

In yet another embodiment, the present invention provides a method for treating fibrosis which involves augmenting the number of dendritic cells in a patient in need of such treatment, wherein the augmented number of dendritic cells is effective for the treatment of fibrosis. In certain embodiments, the number of dendritic cells is augmented in the patient by administering an effective amount for augmenting the dendritic cells of Flt3L. In certain embodiments, the number of dendritic cells is augmented in the patient by administering an effective amount for augmenting the dendritic cells of another cytokine or growth factor suitable for augmenting dendritic cells. In certain other embodiments, the number of dendritic cells is augmented in the patient by administering a cell expressing and secreting an effective amount for treating fibrosis of Flt3L. In yet other embodiments, the number of dendritic cells is augmented in the patient by administering a plasmid carrying the nucleic acid sequence of Flt3L, wherein the plasmid is administered in an amount that is effective for yielding expression of Flt3L in said patient; and wherein the Flt3L is expressed in an amount that is effective for augmenting the number of dendritic cells in said patient

In certain embodiments, the present invention provides methods for the treatment of fibrosis involving administering to a patient in need of such treatment dendritic cells expanded in vitro. In certain embodiments, the dendritic cells may be expanded from bone marrow cells in vitro. In certain other embodiments, the dendritic cells may be expanded from CD34⁺ positive progenitor cells or any other source of stem cells useful for being differentiated into dendritic cells in vitro. In certain embodiments, the dendritic cells are expanded using Flt3L. In certain other embodiments, the dendritic cells are expanded using another cytokine or growth factor suitable for expanding progenitor cells in vitro.

In certain other embodiments, the present invention provides a pharmaceutical formulation including Flt3L and a pharmaceutical carrier. In a certain other embodiment, a pharmaceutical formulation of the invention includes a dendritic cell expanded from CD34⁺ progenitor cells treated with Flt3L and, optionally, Flt3L. In still another embodiment, a pharmaceutical formulation provided by the present invention includes Flt3L, and, optionally, a dendritic cell, or a dendritic cell expanded from CD34⁺ progenitor cells treated with Flt3L, and further includes another cytokine or growth factor. In another embodiment, a pharmaceutical formulation provided by the present invention, further contains a pharmaceutical carrier. In any of the above embodiments, Flt3L may be substituted with another cytokine or growth factor useful for the expansion of dendritic cells in vivo or in vitro.

In another embodiment, the present invention provides a method for treating fibrosis which involves administering to a patient in need of such treatment a plasmid having the nucleic acid sequence of Flt3L, wherein the plasmid is administered in an amount that is effective for yielding expression of Flt3L in said patient; and wherein the Flt3L is expressed in an amount that is effective for the treatment of fibrosis.

In a certain embodiment, the present invention provides a method for the treatment of fibrosis which involves administering to a patient in need of such treatment an effective amount for treating fibrosis of a pharmaceutical formulation and a pharmaceutical carrier provided by the invention.

In yet another embodiment, the present invention provides a method for the treatment of fibrosis afflicting an organ or tissue selected from the group consisting of pancreas, lung, heart, nervous system, bone marrow, lymph nodes, endomyocardium, and retroperitoneum, which involves administering to a patient in need of such treatment an effective amount for treating fibrosis of a pharmaceutical formulation provided in the present invention.

In still another embodiment, the present invention provides a method for the treatment of a disease or condition that is a member selected from the group consisting of cirrhosis, diffuse parenchymal lung disease, post-vasectomy pain syndrome, tuberculosis, sickle-cell anemia, rheumatoid arthritis, progressive massive fibrosis, idiopathic pulmonary fibrosis, injection fibrosis, renal fibrosis, myelofibrosis, cardiac fibrosis, pancreatic fibrosis, skin fibrosis, scleroderma, intestinal fibrosis or strictures, and mediastinal fibrosis, which involves administering to a patient in need of such treatment an effective amount for treating the disease or condition of a pharmaceutical formulation provided in the present invention.

In any of the embodiments described herein, the patient or mammal may be a human.

In any of the embodiments described herein, Flt3L may be isolated or recombinant protein, a biologically active polypeptide fragment of Flt3L, or a mutant or variant of Flt3L.

In certain of the embodiments described above, the present invention provides methods for the treatment of hepatic or pulmonary fibrosis. In certain of the embodiments described herein, the present invention provides methods for the treatment of fibrosis, wherein the fibrosis is afflicting any of the organs or tissues selected from the group consisting of liver, pancreas, lung, heart, nervous system, skin, kidneys, bone marrow, lymph nodes, endomyocardium, and retroperitoneum, and wherein the method of treatment may involve administering to a mammal or a patient in need of such treatment Flt3L, or a dendritic cell, or a dendritic cell expanded from CD34+ progenitor cells treated with Flt3L or another suitable cytokine or growth factor. In yet other embodiments, a method for treating fibrosis afflicting any of the organs or tissues described above involves administering to a mammal or a patient in need of such treatment Flt3L and another cytokine or growth factor.

### BRIEF DESCRIPTION OF THE DRAWINGS

FIG. 1 is a graph showing the fold change over untreated DCs of MMP-9, MMP-10, MMP-14, TIMP-2 and MMP-13 mRNA expression in LPS-treated DCs isolated from Flt3L-treated mice.

FIG. 2A is a Western blot showing MMP-9 protein levels in the culture supernatants of DCs isolated from Flt3L-treated mice following 12, 24, or 36 hours of culture with or without LPS.

FIG. 2B is a Western blot showing MMP-9 protein levels in the cell lysates of DCs isolated from Flt3L-treated mice following 12, 24, or 36 hours of culture with or without LPS.

FIG. 3A shows liver sections stained with Sirius Red to identify the presence of collagen in CCL4-treated mice treated without or with Flt3L (+Flt3L DC Expansion).

FIG. 3B is a graph quantitating the amount of fibrosis resolution in CCL4-treated mice that were not treated with Flt3L (spontaneous resolution) and in CCL4-treated mice also treated with Flt3L (+Flt3L DC expansion).

FIG. 4A shows levels of stellate cell activation by immunohistochemistry for α-smooth muscle actin in liver sections of CCL4-treated mice treated without (CCL4) or with Flt3L (+Flt3L DC expansion).

FIG. 4B is a graph quantitating the percent of cells per field of view that stain positive for α-smooth muscle actin in the livers of CCL4-treated mice treated without (CCL4) or with Flt3L (+Flt3L DC expansion).

FIG. 5 is a Western blot showing the protein expression of MMP-9 and GAPDH in the livers of CCL4-treated mice that were not treated with Flt3L (CCL4), and in CCL4-treated mice that were also treated with Flt3L (CCL4+Flt3L), 3 days after the last CCL4 dose.

FIG. 6 shows expression of MMP-9 protein by immunofluorescence staining of liver sections from CCL4-treated mice treated with (+Flt3L) or without (CCL4) Flt3L.

FIG. 7 is a graph quantitating the amount of collagen assessed by morphometry after Sirius Red staining at 4, 8, and 12 days after the last CCL4 dose, expressed as a percent of total area within the field of view.

FIG. 8 is a graph quantitating the amount of collagen from the livers of CD11c-DTR transgenic mice treated with (DC depletion) or without (Non-depleted) diphtheria toxin to deplete liver DCs during fibrosis resolution, 4 days after last CCL4 dose.

FIG. 9 is graph quantitating the expression of α-smooth muscle actin protein by immunohistochemistry morphometry in the livers of CD11c-DTR transgenic mice treated with (DC depletion) or without (Non-depleted) diphtheria toxin to deplete liver DCs during fibrosis resolution 4 days after last CCL4 dose.

FIG. 10A shows liver sections stained with Sirius Red to identify the presence of collagen in the livers of CCL4-treated mice following transfer of high dose (HD) DCs, low dose (LD) DCs, or no transfer of DCs (CCL4 only), and in the livers of untreated mice (normal (untreated)).

FIG. 108 is a graph quantitating the percent Sirius Red positive liver cells per area in the livers of CCL4-treated mice following transfer of high dose (HD) DCs, low dose (LD) DCs, or no transfer of DCs (CCL4 only), and in the livers of untreated mice (normal (untreated)).

FIG. 11A shows staining of α-smooth muscle actin in the livers of CCL4-treated mice following transfer of high dose (HD) DCs, low dose (LD) DCs, or no transfer of DCs (CCL4 only), and in the livers of untreated mice (normal (untreated)).

FIG. 11B is a graph quantitating the staining of α-smooth muscle actin in the livers of CCL4-treated mice following transfer of high dose (HD) DCs, low dose (LD) DCs, or no transfer of DCs (CCL4 only), and in the livers of untreated mice (normal (untreated)).

FIG. 12 is a graph showing the number of dendritic cells in the liver 12, 36, or 60 hours after administration of DT.

### DETAILED DESCRIPTION

The following descriptions and definitions are provided for clarity and illustrative purposes only, and are not intended to limit the scope of the invention.

The present methods encompass in part a technique for the treatment of fibrosis using fms-like tyrosine kinase 3 ligand (Flt3L). The results of Examples 1 to 4 demonstrate that the methods of the present invention result in an increased rate of fibrosis regression in a mouse model of CCL4-induced liver fibrosis following treatment with Flt3L.

In certain embodiments, the present invention provides a method for treating fibrosis in which systemic treatment with Flt3L results in regression of fibrosis. As shown in the present Examples, Flt3L treatment leads to decreased collagen in the liver and decreased numbers of activated stellate cells in the liver. In certain embodiments, the methods of the invention allow for the expansion of a patient's own dendritic cells in vivo by administering Flt3L. The administration of Flt3L directly to a patient can be exploited to fully expand the population of resting DCs in lymphoid organs and liver [Maraskovsky, E., et al., (1996) J Exp Med 184:1953-62; Shurin, G. V., et al., (2004) Exp Gerontol 39:339-48; Gregory, S. H., et al., (2001) Cytokine 13:202-8], and has already been incorporated in human trials of cancer immunotherapy [Fong, L., et al., (2001) Proc Natl Acad Sci USA 98:8809-14; Disis, M. L., et al., (2002) Blood 99:2845-50].

In other embodiments, the present invention provides methods for obtaining a large number of a patient's own dendritic cells (DCs) using Flt3L-mediated expansion of CD34+ progenitor cells or bone marrow cells in vitro to make large numbers of DCs, and transferring these Flt3L-expanded DCs to the same or a different patient, which enhances recovery from fibrosis. In another embodiment, the invention provides methods for expanding a patient's own population of DCs by administering a cell secreting Flt3L to the patient. These methods are useful for treating fibrosis.

In certain embodiments, Flt3L is administered as a protein, a protein fragment thereof, or a mutant or variant of Flt3L. In other embodiments, Flt3L is administered using hydrodynamic gene therapy, or as a cell secreting endogenous Flt3L or engineered to secrete exogenous Flt3L.

In certain embodiments, the present invention relates to a method for the treatment of liver fibrosis using DCs expanded with Flt3L. The Examples show that transfer of Flt3L-expanded dendritic cells provide beneficial results in a murine model of liver fibrosis. Murine models of hepatic (liver) fibrosis closely mimic characteristics of human hepatic fibrosis and provide a useful tool for understanding human hepatic fibrosis as well as fibrosis affecting other organs and tissues. The terms “liver” and “hepatic” are used interchangeably herein.

In certain other embodiments, the present invention is also useful for the treatment of fibrosis in other organs and tissues, including, for example, pancreas, lung, heart, nervous system, skin, kidneys, bone marrow, lymph nodes, endomyocardium, and retroperitoneum. Diseases associated with fibrosis in these organs and tissues include, but are not limited to, diffuse parenchymal lung disease, post-vasectomy pain syndrome, tuberculosis, sickle-cell anemia, rheumatoid arthritis, progressive massive fibrosis, idiopathic pulmonary fibrosis, renal fibrosis, myelofibrosis, cardiac fibrosis, pancreatic fibrosis, skin fibrosis, scleroderma, intestinal fibrosis or strictures, and mediastinal fibrosis. Fibrosis in all of these organs and tissues is characterized by the formation of excess fibrous connective tissue and can benefit from treatment according to the methods provided in the present invention.

In particular, pulmonary fibrosis, characterized by excessive deposition of fibrotic tissue in the pulmonary interstitium, may also be treated by methods according to the present invention. In certain embodiments, Flt3L is administered to a mammal having pulmonary fibrosis. Administration of Flt3L leads to increased fibrotic regression in the lung, i.e. a lessening of the excessive amount of fibrotic tissue. In certain other embodiments, Flt3L-expanded DCs are transferred to a mammal having pulmonary fibrosis. In yet other embodiments, the Flt3L-expanded DCs are derived from the mammal's CD34⁺ precursor cells. In yet other embodiments, the mammal is a human patient.

It has presently been discovered that Flt3L-expanded liver DCs upregulate MMPs in vitro following exposure to lipopolysaccharide (LPS). MMPs are essential to the degradation of ECM, which is an important process for fibrosis regression. TLR ligands, such as LPS, initiate an inflammatory response in DCs, and thus represent useful adjuvants for determining how DCs respond in a disease state such as hepatic fibrosis, which is also associated with inflammatory responses [Friedman (2008) Gastroenterology; 134: 1655-1669]. Furthermore, it is presently shown that TIMP-2 mRNA is downregulated in liver DCs, further suggesting that DCs contribute to increased fibrosis regression, in part through down-regulating inhibitors of MMPs.

An aspect of the present invention concerns the therapeutic application of Flt3L to treat hepatic fibrosis. In the course of developing the methods of the present invention, a mouse model of hepatic fibrosis was utilized. In this model, hepatic fibrosis was produced in adult mice by administering CCL4 for 8-12 weeks. Herein, these mice are referred to as “CCL4 mice.” The CCL4 mice develop a liver condition having the hallmarks of human fibrosis, including increased collagen in the liver and increased numbers of activated hepatic stellate cells. The CCL4 mice were tested for the rate of fibrosis regression with or without treatment with Flt3L. The data show that Flt3L increases the rate of fibrosis regression, which has not been previously shown. Hence, this is an unexpected action of Flt3L treatment in vivo. The methods and outcomes associated with CCL4-induced hepatic fibrosis in a rodent model of hepatic fibrosis are described in detail in Proctor, E. et al. (1982) 83:1183-1190, which is hereby incorporated by reference in its entirety.

It has also been discovered that DCs in CCL4 mice treated with Flt3L have increased expression of MMP-9 protein. MMPs are important in fibrosis regression, indicating that DCs likely play a direct role in breakdown of the ECM and in fibrosis regression. In another set of experiments, it was discovered that CCL4 mice receiving transferred Flt3L-expanded DCs have increased rates of fibrosis regression, indicating that Flt3L-expanded DCs can mediate fibrosis regression upon transfer into a patient having hepatic fibrosis. The role of Flt3L-expanded DCs in fibrosis regression has not been previously shown, and thus, is an unexpected action of Flt3L treated DCs in vivo.

In has also been shown that DCs are critical to fibrosis regression using B6.FVB-Tg(Itgax-DTR/EGFP)57Lan/J (“CD11c-DTR”) transgenic mice. Normal mice do not express diphtheria toxin (DT) receptor and are unaffected by exposure to DT. CD11c-DTR transgenic mice express the human DT receptor (DTR) under the control of the CD11c promoter, which is expressed specifically and constitutively on DCs. Thus, DCs in CD11c-DTR transgenic mice specifically express the DTR and, upon exposure to DT, rapidly undergo apoptosis, thereby depleting the DT-treated mouse of DCs. Not all classes of DCs are depleted, however; only the classical DCs that express high level of MHC class II are depleted (“activated DCs”). The number of, another subset of DCs, plasmacytoid DCs, is not changed by DT administration [see Probst, H. C., et al., (2005) Clin Exp Immunol. 14: 398-404; Probst, H. C. and M. van den Broek, (2005) J. Immunol. 174: 3920-4; Bennett, C. L. and B. E. Clausen (2007) Trends Immunol. 28:525-31]. Thus, this transgenic mouse system represent a useful method to assess the specific role of classical DCs in hepatic fibrosis, and may also be extrapolated for fibrosis affecting other organs, since fibrosis in other organs has a similar phenotype. In CD11c-DTR transgenic mice treated with CCL4 (“CCL4 CD11c-DTR transgenic mice”), DT treatment and depletion of DCs results in decreased rates of fibrosis regression and decreased levels of MMPs in the liver, further indicating that DCs play a critical role in resolution of fibrosis.

Methods of the present invention can be used in any condition where it would be beneficial for the reducing of excessive amounts of fibrotic tissue in an organ or tissue.

Flt3L is a cytokine that, when administered systemically, can increase the numbers of circulating DCs more than 40-fold, and human DCs stimulated by administration of Flt3L have been shown to be functional in vitro [Disis, M L et al. (2002) Blood. 99: 2845-2850]. As used herein, the term “Flt3L” refers to a genus of polypeptides that are described in U.S. Pat. No. 5,554,512, incorporated herein by reference. A human Flt3L cDNA was deposited with the American Type Culture Collection, Rockville, Md., USA (ATCC) on Aug. 6, 1993 and assigned accession number ATCC 69382. The deposit was made under the terms of the Budapest Treaty. Flt3L can be made according to the methods described in the documents cited above.

The full-length human Flt3L protein has been described and has protein accession number NP_{—}004110 (SEQ ID NO: 1). The mouse Flt3L protein has also been described and has protein accession number NP_{—}034359 (SEQ ID NO: 3). Coding sequences for Flt3L include accession numbers NM_{—}004119 (human, SEQ ID NO: 2) and NM_{—}010229 (murine, SEQ ID NO: 4). Any active fragments of Flt3L proteins, as well as full-length Flt3L proteins, variants, and mutants of Flt3L are also contemplated in the present invention.

During hepatic fibrosis, decreased portal blood flow, induced by both constricting individual sinusoids and by contracting of the cirrhotic liver, leads to loss of liver function and, ultimately, can lead to patient death. While recent studies have suggested that fibrosis and late-stage fibrosis (cirrhosis) can be reversible, methods of achieving reversal of the disease in humans are not well known. One possible therapeutic approach would be to administer DCs to induce regression of fibrosis; however, presently, this approach is not feasible because DCs are rare cells in humans constituting less than 1% of circulating white blood cells [Disis, M L et al. (2002) Blood. 99: 2845-2850]. Moreover, the transfer of DCs to a recipient from an exogenous source is not ideal, because the recipient's immune system would attack and deplete the foreign, transferred DCs before they could provide any therapeutic benefit to the recipient.

The present invention involves methods that allow for the expansion of a patient's own dendritic cell population for the treatment of hepatic fibrosis. Specifically, the invention uses Flt3L to expand a patient's DC population in vivo or in vitro. Also contemplated in the present invention are other methods suitable for the expansion of dendritic cells in vivo or in vitro, such as culturing dendritic cells with other cytokines or growth factors that have a similar effect on dendritic cells as Flt3L. These methods have many advantages, including increased regression of liver fibrosis, as measured by decreased levels of collagen in fibrotic livers, decreased numbers of activated stellate cells (the cells associated with fibrosis), and increased expression of MMPs by DCs (enzymes which are critical to the breakdown of ECM, leading to fibrosis regression).

The full-length protein sequences of human MMPs have been described, and have protein accession numbers: NP_{—}002412 (MMP-1; SEQ ID NO: 6), NP_{—}004985 (MMP-9; SEQ ID NO: 8), NP_{—}002416 (MMP-10; SEQ ID NO: 10), and NP_{—}004986 (MMP-14; SEQ ID NO: 12). The coding sequences for human MMPs have also been described, and have gene accession numbers: NM_{—}002421 (MMP-1; SEQ ID NO: 5), NM_{—}004994 (MMP9; SEQ ID NO: 7), NM_{—}002425 (MMP 10; SEQ ID NO: 9), NM_{—}004995 (MMP14; SEQ ID NO: 11). The protein and coding sequences for human TIMP-2 are also described and have protein accession number NP_{—}003246 (SEQ ID NO: 14) and gene accession number NM_{—}003255 (SEQ ID NO: 13). The protein and coding sequences for human TIMP-1 are also described and have protein accession number NP_{—}003245 (SEQ ID NO: 43) and gene accession number NM_{—}003254 (SEQ ID NO: 44).

The full-length protein sequences of murine (mus musculus) MMPs have been described, and have protein accession numbers: NP_{—}038627 (MMP-9; SEQ ID NO: 16), NP_{—}062344 (MMP-10; SEQ ID NO: 18), NP_{—}032633 (MMP-13; SEQ ID NO: 20), and NP_{—}032634 (MMP-14; SEQ ID NO: 22). The coding sequences for murine MMPs have also been described, and have gene accession numbers: NM_{—}013599 (MMP-9; SEQ ID NO: 15), NM_{—}019471 (MMP-10; SEQ ID NO: 17), NM_{—}008607 (MMP-13; SEQ ID NO: 19), and NM_{—}008608 (MMP-14; SEQ ID NO: 21). The protein and coding sequences for murine TIMP-2 are also described and have protein accession number NP_{—}035724 (SEQ ID NO: 24) and gene accession number NM_{—}011594 (SEQ ID NO: 23). The protein and coding sequences for murine TIMP-1 are also described and have protein accession number NP_{—}001037849 (SEQ ID NO: 45) and gene accession number NM_{—}001044384 (SEQ ID NO: 46).

Moreover, the invention provides for the use of an effective amount of Flt3L to increase or mobilize the numbers of intermediate cells in vivo, for example, in the patient's peripheral blood or spleen. While the invention relates to the generation of large numbers of such downstream and intermediate cells (e.g., myeloid cells, monocytic cells, macrophages and NK cells) from CD34⁺ cells using Flt3L, the focus is particularly on dendritic cells. By increasing the quantity of the patient's dendritic cells, such cells may themselves be used to treat hepatic fibrosis. Flt3L may be used; therefore, to increase the numbers of dendritic cells in vivo to increase the rate of regression of liver fibrosis.

In certain embodiments, the Flt3L may be isolated protein or recombinant protein. In some embodiments, an effective amount of isolated or recombinant Flt3L protein may be administered to a mammal or a patient by any suitable means of administration.

The invention further provides for using combination therapy to enhance a patient's recovery from hepatic fibrosis. Such combination therapy includes administering Flt3L and one or more therapeutic reagents or growth factors, such as, e.g., GM-CSF or M-CSF in amounts sufficiently effective to increase the rate of fibrotic regression. Alternatively, Flt3L may be used to differentiate a patient's CD34⁺ progenitor cells into DCs in vitro. These in vitro generated DCs may then be administered to the patient in order to treat hepatic fibrosis without generating an immune response associated with transplant rejection, since the transferred DCs are derived from the patient's own cells.

For the growth and culture of dendritic cells, a variety of growth and culture media can be used, and the composition of such media can be readily determined by a person having ordinary skill in the art. Suitable growth media are solutions containing nutrients or metabolic additives, and include those that are serum-depleted or serum-based. Representative examples of growth media are RPMI, TC 199, Iscoves modified Dulbecco's medium [Iscove, et al., (1978) J. Exp. Med. 147:923], DMEM, Fischer's, alpha medium, NCTC, F-10, Leibovitz's L-15, MEM and McCoy's. Particular examples of nutrients that will be readily apparent to the skilled artisan include, serum albumin, transferrin, lipids, cholesterol, a reducing agent such as 2-mercaptoethanol or monothioglycerol, pyruvate, butyrate, and a glucocorticoid such as hydrocortisone 2-hemisuccinate. More particularly, the standard media includes an energy source, vitamins or other cell-supporting organic compounds, a buffer such as HEPES or Tris, which acts to stabilize the pH of the media, and various inorganic salts. Particular reference is made to PCT Publication No. WO 95/00632, wherein a variety of serum-free cellular growth media is described; such disclosure is incorporated herein by reference.

For any of the ex vivo methods of the invention, peripheral blood progenitor cells (PBPC) and peripheral blood stem cells (PBSC) are collected using apheresis procedures known in the art. See, for example, Bishop et al. (1994) Blood. 83:610 616]. Briefly, PBPC and PBSC are collected using conventional devices, for example, a Haemonetics® Model V50 apheresis device (Haemonetics, Braintree, Mass.). Four-hour collections are performed typically no more than five times weekly until, for example, approximately 6.5×10⁸ mononuclear cells (MNC)/kg patient are collected. The cells are suspended in standard media and then centrifuged to remove red blood cells and neutrophils. Cells located at the interface between the two phases (also known in the art as the buffy coat) are withdrawn and resuspended in HBSS. The suspended cells are predominantly mononuclear and a substantial portion of the cell mixture are early stem cells. The resulting stem cell suspension then can be contacted with biotinylated anti-CD34 monoclonal antibodies or other cell-binding means. The contacting period is maintained for a sufficient time to allow substantial interaction between the anti-CD34 monoclonal antibodies and the CD34 antigens on the stem cell surface. Typically, times of at least one hour are sufficient. The cell suspension then is brought into contact with the isolating means provided in the kit.

The isolating means can comprise a column packed with avidin-coated beads. Such columns are well known in the art, see Berenson, et al. (1986) J. Cell Biochem. 10D:239. The column is washed with a PBS solution to remove unbound material. Target stem cells can be released from the beads and from anti-CD34 monoclonal antibody using conventional methods. The stem cells obtained in this manner can be frozen in a controlled rate freezer (e.g., Cryo-Med®, Mt. Clemens, Mich.), then stored in the vapor phase of liquid nitrogen. Ten percent dimethylsulfoxide can be used as a cryoprotectant. After all collections from the donor have been made, the stem cells are thawed and pooled. Aliquots containing stem cells, growth medium, such as McCoy's 5A medium, 0.3% agar, and at least one of the expansion factors: recombinant human GM-CSF, IL-3, recombinant human Flt3L, and recombinant human GM-CSF/IL-3 fusion molecules (PIXY321) at concentrations of approximately 200-U/mL, are cultured and expanded at 37° C. in 5% CO₂ in fully humidified air for 14 days. Optionally, human IL-1α or IL-4 may be added to the cultures. Certain combinations of expansion factors comprising Flt3L plus, e.g., either IL-3 or a GM-CSF/IL-3 fusion protein are also contemplated.

For in vivo administration to a patient, such as a mammal, e.g, a human patient, dendritic cells of the present invention may be administered by parenteral route. The term “parenteral” includes subcutaneous injections, intravenous, intramuscular, intracisternal injection, or infusion techniques. The dendritic cells may be administered in any suitable preparation. For injection or infusion techniques, the dendritic cells of the invention may be suspended in any suitable injection buffer, such as, but not limited to PBS or PBS containing anti-coagulants.

The compositions of the invention containing dendritic cells will typically contain an effective amount of dendritic cells, alone, or in combination with an effective amount of any other active material, e.g., Flt3 ligand, GM-CSF, or M-CSF. Effective amounts, or dosages, and desired concentrations of dendritic cells contained in the compositions may vary depending upon many factors, including the intended use, patient's body weight and age, and route of administration. The suitable route of administration of dendritic cells is parenteral. Preliminary doses can be determined according to animal tests, and the scaling of dosages for human administration can be performed according to art-accepted practices. Keeping the above description in mind, typical dosages (effective amounts) of dendritic cells for administration to a patient may range from 1×10³ to 1×10⁸ cells per dose, although more or less cells may be used. Preferably the number of dendritic cells ranges from 1×10⁴ to 1×10⁸, more preferably from 1×10⁵ to 1×10⁸, still more preferably from 1×10⁶ to 1×10⁸, and most preferably from 1×10⁶ to 1×10⁷. However, other ranges are possible, depending on a patient's response. The number and frequency of doses may also be determined based on the patient's response to administration of the composition, e.g., if the patient's symptoms improve and/or if the patient tolerates administration of the composition without adverse reaction; in some patients, a single dose is sufficient, other patients may receive a weekly, biweekly, or monthly administration of the dendritic cell-containing composition of the invention. The duration of treatment will depend upon the patient's response to treatment, i.e., if the patient's condition improves. For example, if the patient has liver fibrosis, improvement in liver function may be determined e.g. by blood tests or other routine methods in the art, and dosing and duration of treatment may be scaled based on the patient's individual response to treatment.

In compositions of the invention containing Flt3L, dendritic cells, and/or cells secreting Flt3L, one or more additional growth factors or cytokines, e.g., GM-CSF or M-CSF may be included in effective amounts in the composition or coadministered with the composition by any suitable route and method of administration. The amount of the additional growth factor or cytokine is typically in the range of from about 10 μg/kg to about 100 μg/kg. A preferred dose range is on the order of about 10 μg/kg to about 20 μg/kg.

Flt3L can be administered topically, parenterally, or by inhalation. These compositions will typically contain an effective amount of the Flt3L, alone or in combination with an effective amount of any other active material, e.g., those described above. Effective amounts, or dosages, and desired concentrations of Flt3L, contained in the compositions may vary depending upon many factors, including the intended use, patient's body weight and age, and route of administration. Preliminary doses can be determined according to animal tests, and the scaling of dosages for human administration can be performed according to art-accepted practices. Keeping the above description in mind, typical dosages of Flt3L may range from about 10 μg/kg to about 100 μg/kg. A preferred dose range is on the order of about 10 μg/kg to about 20 μg/kg. In certain embodiments, a patient may receive, for example, 20 μg/kg of Flt3L per day subcutaneously for 14 days each month [see Disis, M L et al. (2002) Blood. 99: 2845-2850].

In certain embodiments, Flt3L can be administered using cells that are engineered to express and secrete Flt3L protein. The Flt3L-expressing cell may be administered by any suitable route to a patient in order to treat hepatic fibrosis. For example, B16 melanoma cells transduced with retroviral vector containing the Flt3L DNA sequence may be injected subcutaneously to deliver a continuous, systemic supply of Flt3L protein. Mice injected with the retrovirally transduced Flt3L B16 melanoma cells have dramatic alterations in hematopoiesis, with total white blood cell counts reaching up to 17,000 (×10⁻³/ml) by day 14 after injection [see, Mach, N. et al. (2000) Cancer Research. 60:3239-3246]. In yet other embodiments, any type of cell that is engineered to secrete Flt3L protein may be used as described above.

In still other embodiments, Flt3L can be administered using hydrodynamic gene therapy, or naked DNA gene transfer. Using this method, naked DNA can be delivered to cells in vivo and results in gene expression. Recent studies have shown that naked plasmid DNA (pDNA) can be delivered efficiently to cells in vivo either via electroporation, or by intravascular delivery, and has great prospects for gene therapy [Herweijer, H. and Wolff, J. A. (2003) Gene Therapy; 10:453-458]. Studies have shown that tail vein pDNA delivery is a simple and effective method for transfecting liver cells in mice and rats. The tail vein drains into the vena cava. Delivery of a large bolus may result in a liquid volume in the vena cava that is too large for the heart to handle rapidly. The fluids back up and end up predominantly in the liver, resulting in gene transfer. Several groups have found that the optimal volume is about 10% of the body weight of a mouse or rat. The delivery time is approximately between 5 and 7 seconds in the mouse and 15-20 seconds in the rat. Tail vein (or hydrodynamic gene delivery) results in very high levels of gene transfer. Typically, 10-15% of the hepatocytes are transfected in mouse liver following injection of 10 μg DNA, but levels up to 40% have been reported, one day after gene delivery. It is easy to regulate the level of gene expression in the recipient by adjusting the amount of pDNA that is administered to the recipient [Herweijer, H. and Wolff, J. A. (2003) Gene Therapy; 10:453-458].

While the liver is the organ that is predominantly transfected by this hydrodynamic gene therapy approach, the liver may also be used as a site of ectopic expression for secreted proteins (such as, e.g., Flt3L). Thus, the method of the present invention is useful for treating diseases at other sites or organs in the body, in addition to liver, since injection of pDNA containing the nucleic acid sequence of Flt3L can result in Flt3L being secreted and disseminated systemically. Gene transfer efficiencies similar to those reported for rodents have been reported in larger animals and mammals, including rabbits, dogs and monkeys. Thus, this method is also expected to be useful in humans [Herweijer, H. and Wolff, J. A. (2003) Gene Therapy; 10:453-458].

Other methods of gene transfer that are contemplated in the present invention include intravascular delivery of pDNA or direct injection of pDNA into skeletal muscle. Intravascular delivery results in the dissemination of the gene throughout the tissue, since the vascular system accesses every cell. Vascular delivery may be systemic or regional in which injections are into specific vessels that supply a target or tissue. For example, pDNA containing Flt31, may be injected directly into the portal vein, the hepatic vein, or the bile duct in mice and rats, in order to obtain efficient transgene expression in hepatocytes. Such injections can be done via catheters in humans as well, making this a relatively simple procedure for use in humans [Herweijer, H. and Wolff, J. A. (2003) Gene Therapy; 10:453-458].

The approach for hydrodynamic gene therapy described in the Examples section herein has been described in detail in He, Y., et al. (2000) Hum Gene Ther. 11:547-54, which is herein incorporated by reference in its entirety. Briefly, the plasmids pNGVL-hFLex along with a control plasmid, may be obtained from the National Gene Vector Laboratory (University of Michigan, Ann Arbor, Mich.). The plasmid pNGVL-hFLex consists of the pNGVL eukaryotic gene expression plasmid into which the extracellular domain (amino acids 1-182) of human Flt3L, including the secretion signal, is inserted downstream of the cytomegalovirus (CMV) promoter and intron. It has been reported that in vitro transfection of HEK 293 cells with this construct results in significant levels of human Flt3L in both cell lysates and the supernatants of transfected cells, indicating that human Flt3L expressed from the pNGVL-hFLex plasmid can be secreted [see, He, Y., et al. (2000) Hum Gene Ther. 11:547-54]. When mice are injected with 10 μg of the pNGVL-hFLex plasmid, serum level of 1.12±0.23 μg/ml of human Flt3L is detected 4 hours after injection, and a peak serum level of 39.12±12.78 μg/ml is detected after 24 hours. Serum levels of human Flt3L are maintained above 1 μg/ml up to day 6 and then decrease dramatically by day 8. Using this method, increased number of DCs as well as NK cells in the lymph nodes and spleen can be observed beginning on day 5, peaking between days 8 and 12, and returning to normal numbers by day 20. The DCs and NK cells are functional, as has been shown by mixed leukocyte reactions and lysis of YAC-1 cells, respectively.

In yet other embodiments, viral vectors, derived from viruses such as, but not limited to Adeno-associated virus, adenoviruses, lentivirus, herpes simplex virus, Sendai virus, retroviruses, DNA viruses, and mutants of any of the above, may be used to administer Flt3L to a mammal (i.e., for gene transfer). Adeno-associated virus (AAV) is particularly attractive for gene transfer because it does not induce any pathogenic response and can integrate into the host cellular chromosome [Kotin et al. (1990) Proc. Natl. Acad. Sci. USA, 87:2211-2215). AAV, a parvovirus, is a ubiquitous virus (antibodies are present in 85% of the U.S. human population) that has not been linked to any disease. The AAV terminal repeats (TRs) are the only essential cis-components for the chromosomal integration [Muzyczka and McLaughin (1988) Current Communications in Molecular Biology: Viral Vectors, Glzman and Hughes Eds., Cold Spring Harbor Laboratory Press, Cold Spring Harbor, N.Y., pp. 39-44]. These TRs are reported to have promoter activity [Flotte et al. (1993) Proc. Natl, Acad. Sci. USA, 90(22):10613-10617]. They may promote efficient gene transfer from the cytoplasm to the nucleus or increase the stability of plasmid DNA and enable longer-lasting gene expression [Bartlett et al. (1996) Cell Transplant., 5(3):411-419]. AAV-based plasmids have been shown to drive higher and longer transgene expression than the identical plasmids lacking the TRs of AAV in most cell types (Philip et al., 1994; Shafron et al., 1998; Wang et al., 1999). The benefits and methods associated with AAV for use in gene therapy are described in detail in U.S. Pat. No. 7,342,111 to Lewin et al., and is hereby incorporated by reference in its entirety.

In still other embodiments, any method for delivering DNA to a recipient that is known in the art is an acceptable form of delivery of Flt3L gene. For example, naked DNA may be covered in lipids or cationic lipids, such as in a micelle or liposome before injection into a recipient. DNA may be complexed with polymers, such as poly(ethylene glycol) to form polyplexes. In other embodiments, liposomes and inactivated virus, such as HIV or influenza virus may be combined in a virosome with the DNA to be transferred.

Pharmaceutical Compositions, Formulations and Administration

While it is possible to use a composition provided by the present invention for therapy as is, it may be preferable to administer it in a pharmaceutical formulation, e.g., in admixture with a suitable pharmaceutical excipient, diluent, or carrier selected with regard to the intended route of administration and standard pharmaceutical practice. Accordingly, in one aspect, the present invention provides a pharmaceutical composition or formulation comprising at least one active composition of the invention, or a pharmaceutically acceptable derivative thereof, in association with a pharmaceutically acceptable excipient, diluent, and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. In one embodiment, the active ingredient (e.g., Flt3L, dendritic cells, and/or cells secreting Flt3L) can be delivered in a vesicle, including as a liposome (see Langer, Science, 1990; 249:1527-1533; Treat et al., in Liposomes in the Therapy of Infectious Disease and Cancer, Lopez-Berestein and Fidler (eds.), Liss: New York, pp. 353-365 (1989); Lopez-Berestein, ibid., pp. 317-327; see generally ibid.).

For in vivo administration to humans, the compositions of the invention, such as those containing effective amounts of Flt3L, dendritic cells, and/or cells secreting Flt3L, can be formulated according to known methods used to prepare pharmaceutically useful compositions. The Flt3L, dendritic cells, and/or cells secreting Flt3L can be combined in admixture, either as the sole active material or with other known active materials, as described supra (e.g., GM-CSF or M-CSF), with pharmaceutically suitable diluents (e.g., Tris-HCl, acetate, phosphate), preservatives (e.g., Thimerosal, benzyl alcohol, parabens), emulsifiers, solubilizers, adjuvants and/or carriers. Suitable carriers and their formulations are described in Remington's Pharmaceutical Sciences, 16th ed. 1980, Mack Publishing Co. In addition, such compositions can contain Flt3L complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Such compositions will influence the physical state, solubility, stability, rate of in vivo release, and rate of in vivo clearance of Flt3L.

The effective amounts of compounds, compositions including pharmaceutical formulations of the present invention include doses that partially or completely achieve the desired therapeutic, prophylactic, and/or biological effect. In a specific embodiment, an effective amount of Flt3L, dendritic cells, and/or cells secreting Flt3L administered to a patient having fibrosis, e.g., liver fibrosis, is effective for reducing or curing the fibrosis in the patient. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

When formulated in a pharmaceutical composition or formulation, a therapeutic compound of the present invention can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice, which is incorporated herein by reference to the extent of teaching such derivatives. Preferred pharmaceutically acceptable derivatives are salts, solvates, esters, carbamates, and phosphate esters. Particularly preferred pharmaceutically acceptable derivatives are salts, solvates, and esters. Most preferred pharmaceutically acceptable derivatives are salts and esters.

In accordance with the present invention there may be employed conventional molecular biology, microbiology, protein expression and purification, antibody, and recombinant DNA techniques within the skill of the art. Such techniques are explained fully in the literature. See, e.g., Sambrook et al. (2001) Molecular Cloning: A Laboratory Manual. 3^{rd} ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Son, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.; Nucleic Acid Hybridization, Hames & Higgins eds. (1985); Transcription And Translation, Hames & Higgins, eds. (1984); Animal Cell Culture Freshney, ed. (1986); Immobilized Cells And Enzymes, IRL Press (1986); Perbal, A Practical Guide Molecular Cloning (1984); and Harlow and Lane. Antibodies: A Laboratory Manual (Cold Spring Harbor Laboratory Press: 1988).

The electronic version of the sequence listing containing SEQ ID NOs: 1-46 is hereby incorporated by reference in its entirety.

As used herein, the term “antigen presenting cell” refers to a cell that has the ability to present peptide or lipid antigen on surface major histocompatibility complex (MHC) molecules.

The term “growth factor” can be a naturally occurring, endogenous or exogenous protein, or recombinant protein, capable of stimulating cellular proliferation and/or cellular differentiation.

As used herein, the term “Flt3L” refers to a genus of polypeptides that bind and complex independently with Flt3 receptor found on progenitor and stem cells and other hematopoietic cells. The term “Flt3L” encompasses proteins having the amino acid sequence set forth in SEQ ID NO:1 or the amino acid sequence set forth in SEQ ID NO: 3, as well as those proteins having a high degree of similarity or a high degree of identity with the amino acid sequence set forth in SEQ ID NO:1 or the amino acid sequence set forth in SEQ ID NO: 3, and which proteins are biologically active and bind the Flt3 receptor. In addition, the term refers to biologically active gene products of the DNA of SEQ ID NO: 2 or SEQ ID NO: 4. Further encompassed by the term “Flt3L” are the membrane-bound proteins (which include an intracellular region, a membrane region, and an extracellular region), and soluble or truncated proteins which comprise primarily the extracellular portion of the protein, retain biological activity and are capable of being secreted.

The term “biologically active” as it refers to Flt3L, means that the Flt3L is capable of binding to Flt3 receptor. Alternatively, “biologically active” means the Flt3L is capable of transducing a stimulatory signal to the cell through the membrane-bound Flt3 receptor.

The procedure for “ex vivo expansion” of hematopoietic stem and progenitor cells is described in U.S. Pat. No. 5,199,942, incorporated herein by reference. Briefly, the term means a method comprising: (1) collecting CD34⁺ hematopoietic stem and progenitor cells from a patient from peripheral blood harvest or bone marrow fexplants; and (2) expanding such cells ex vivo. In addition to the cellular growth factors described in U.S. Pat. No. 5,199,942, other factors such as Flt3L, IL-1, IL-3, c-kit ligand, can be used.

As used herein, the terms “fibrosis regression”, “fibrotic regression”, “fibrosis resolution” and the like mean an improvement in any stage of the disease state encompassed by “hepatic fibrosis” or “cirrhosis”, including but not limited to decreased levels of collagen in the liver and/or decreased numbers of activated hepatic stellate cells, myofibroblasts, or other mesenchymal cells whether derived from within the liver or extra-hepatic sites.

As used herein, the term “gene transfer” refers to the transfer of genetic material to an organism.

The term “gene therapy” refers to the insertion of genes into an individual's cells and/or tissues to treat a disease. In certain embodiments, a mammal or patient may be administered an effective amount of a plasmid or viral vector containing the nucleic acid sequence of Flt3L to treat fibrosis. An effective amount of a viral vector or plasmid is defined herein as an amount of the viral vector or plasmid that, upon administration to a patient or mammal, results in the expression of an effective amount for treating fibrosis of Flt3L.

### Expression Construct

By “expression construct” is meant a nucleic acid sequence comprising a target nucleic acid sequence or sequences whose expression is desired, operatively associated with expression control sequence elements which provide for the proper transcription and translation of the target nucleic acid sequence(s) within the chosen host cells. Such sequence elements may include a promoter and a polyadenylation signal. The “expression construct” may further comprise “vector sequences”. By “vector sequences” is meant any of several nucleic acid sequences established in the art which have utility in the recombinant DNA technologies of the invention to facilitate the cloning and propagation of the expression constructs including (but not limited to) plasmids, cosmids, phage vectors, viral vectors, and yeast artificial chromosomes.

Expression constructs of the present invention may comprise vector sequences that facilitate the cloning and propagation of the expression constructs. A large number of vectors, including plasmid and fungal vectors, have been described for replication and/or expression in a variety of eukaryotic and prokaryotic host cells. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest.

### Express and Expression

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cells genetic machinery. The gene or sequence may include nonfunctional sequences or sequences with no known function. A host cell that receives and expresses introduced DNA or RNA has been “transformed” and is a “transformant” or a “clone”. The DNA or RNA introduced to a host cell can come from any source, including cells of the same genus or species as the host cell, or cells of a different genus or species.

### Expression System

The term “expression system” means a host cell and compatible vector under suitable conditions, e.g., for the expression of a protein coded for by foreign DNA carried by the vector and introduced to the host cell.

### Gene or Structural Gene

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A coding sequence is “under the control of” or “operatively associated with” expression control sequences in a cell when RNA polymerase transcribes the coding sequence into RNA, particularly mRNA, which is then trans-RNA spliced (if it contains introns) and translated into the protein encoded by the coding sequence.

The term “expression control sequence” refers to a promoter and any enhancer or suppression elements that combine to regulate the transcription of a coding sequence. In a preferred embodiment, the element is an origin of replication.

### Heterologous

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. For example, the present invention includes chimeric DNA molecules that comprise a DNA sequence and a heterologous DNA sequence which is not part of the DNA sequence. A heterologous expression regulatory element is such an element that is operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed. In certain embodiments, heterologous is used to describe a cell that is transferred from one individual to another individual, and is therefore, not isolated from the recipient of the transferred cell.

### Homologous

The term “homologous” as used in the art commonly refers to the relationship between nucleic acid molecules or proteins that possess a “common evolutionary origin,” including nucleic acid molecules or proteins within superfamilies (e.g., the immunoglobulin superfamily) and nucleic acid molecules or proteins from different species (Reeck et al., Cell 1987; 50: 667). Such nucleic acid molecules or proteins have sequence homology, as reflected by their sequence similarity, whether in terms of substantial percent similarity or the presence of specific residues or motifs at conserved positions.

### Host Cell

The term “host cell” means any cell of any organism that is selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or one or more cells in a non-human animal (e.g., a transgenic animal or a transiently transfected animal). Suitable host cells include but are not limited to Streptomyces species and E. coli.

### Treating or Treatment

“Treating” or “treatment” of a state, disorder or condition includes:

(1) preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or

(2) inhibiting the state, disorder or condition, i.e., arresting, reducing or delaying the development of the disease or a relapse thereof (in case of maintenance treatment) or at least one clinical or sub-clinical symptom thereof; or

(3) relieving the disease, i.e., causing regression of the state, disorder or condition or at least one of its clinical or sub-clinical symptoms.

The benefit to a subject to be treated is either statistically significant or at least perceptible to the patient or to the physician.

### Patient or Subject

“Patient” or “subject” refers to mammals and includes human and veterinary subjects.

### Therapeutically Effective Amount

A “therapeutically effective amount” means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

### About or Approximately

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise stated, the term ‘about’ means within an acceptable error range for the particular value.

### Dosage

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level.

### Carrier

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

### Isolated

As used herein, the term “isolated” means that the referenced material is removed from the environment in which it is normally found. Thus, an isolated biological material can be free of cellular components, i.e., components of the cells in which the material is found or produced. Isolated nucleic acid molecules include, for example, a PCR product, an isolated mRNA, a cDNA, or a restriction fragment. Isolated nucleic acid molecules also include, for example, sequences inserted into plasmids, cosmids, artificial chromosomes, and the like. An isolated nucleic acid molecule is preferably excised from the genome in which it may be found, and more preferably is no longer joined to non-regulatory sequences, non-coding sequences, or to other genes located upstream or downstream of the nucleic acid molecule when found within the genome. An isolated protein may be associated with other proteins or nucleic acids, or both, with which it associates in the cell, or with cellular membranes if it is a membrane-associated protein.

### Mutant

As used herein, the terms “mutant” and “mutation” refer to any detectable change in genetic material (e.g., DNA) or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. As used herein, the term “mutating” refers to a process of creating a mutant or mutation.

### Nucleic Acid Hybridization

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3rd ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washing in 0.1×SSC/0.1% SDS at 68° C. (where 1×SSC is 0.15M NaCl, 0.15M Na citrate) or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos)). Accordingly, the term “high stringency hybridization” refers to a combination of solvent and temperature where two strands will pair to form a “hybrid” helix only if their nucleotide sequences are almost perfectly complementary (see Molecular Biology of the Cell, Alberts et al., 3rd ed., New York and London: Garland Publ., 1994, Ch. 7).

Conditions of intermediate or moderate stringency (such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2nd ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3).

As used herein, the term “standard hybridization conditions” refers to hybridization conditions that allow hybridization of sequences having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any desired nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are of about the same length as the particular desired nucleic acid.

### Nucleic Acid Molecule

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

### Orthologs

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity. The present invention encompasses all orthologs of the desired protein.

### Operatively Associated

By “operatively associated with” is meant that a target nucleic acid sequence and one or more expression control sequences (e.g., promoters) are physically linked so as to permit expression of the polypeptide encoded by the target nucleic acid sequence within a host cell.

### Percent Sequence Similarity or Percent Sequence Identity

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al., J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al., Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/on the WorldWideWeb. Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, Mass.; available at accelrys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the invention) is using a Blossum 62 scoring matrix with a gap open penalty of 12, a gap extend penalty of 4, and a frameshift gap penalty of 5.

In addition to the cDNA sequences encoding various desired proteins, the present invention further provides polynucleotide molecules comprising nucleotide sequences having certain percentage sequence identities to any of the aforementioned sequences. Such sequences preferably hybridize under conditions of moderate or high stringency as described above, and may include species orthologs.

### Substantially Similar

The term “substantially similar” means a variant amino acid sequence preferably that is at least 80% identical to a native amino acid sequence, most preferably at least 90% identical.

### Variant

The term “variant” may also be used to indicate a modified or altered gene, DNA sequence, enzyme, cell, etc., i.e., any kind of mutant.

### Kits

In one embodiment, the invention relates to a kit comprising an effective amount of a pharmaceutical formulation comprising Flt3L, and is useful for the treatment of hepatic fibrosis and is packaged in a manner suitable for administration to a patient. In another embodiment, the invention relates to a kit comprising an effective amount of a pharmaceutical formulation comprising a Flt3L-secreting cell, and is useful for the treatment of hepatic fibrosis and is packaged in a manner suitable for administration to a patient. In yet another embodiment, the invention relates to a kit comprising an effective amount of a pharmaceutical formulation comprising a dendritic cell, and is useful for the treatment of hepatic fibrosis and is packaged in a manner suitable for administration to a patient. in some embodiments the dendritic cells are derived from CD34+ progenitor cells or bone marrow cells that have been expanded with Flt3L. In certain embodiments, the kits also include instructions teaching one or more of the methods described herein.

The abbreviations in the specification correspond to units of measure, techniques, properties or compounds as follows: “min” means minutes, “h” means hour(s), “μL” means microliter(s), “mL” means milliliter(s), “mM” means millimolar, “M” means molar, “μl” means microliter(s); “mmole” means millimole(s), “kb” means kilobase, “bp” means base pair(s), a.a. means “amino acid(s)”, and “IU” means International Units. “Polymerase chain reaction” is abbreviated PCR; “Reverse transcriptase polymerase chain reaction” is abbreviated RT-PCR; quantitative reverse transcriptase polymerase chain reaction is abbreviated “qPCR”, “Sodium dodecyl sulfate” is abbreviated SDS. “Flt3L” means fms-like tyrosine kinase 3 ligand, “DC” means dendritic cell, and “MMP” means matrix metalloproteinase.

### EXAMPLES

### Materials and Methods

The following describes the materials and methods employed in Examples 1-4.

### Fibrosis Model

For induction of hepatic fibrosis in mice, CCL4 (Sigma-Aldrich, St. Louis, Mo.) was administered twice weekly at a concentration of 150-200 μl of 10% CCL4 per 100 g (mouse weight) in a 1:1 ratio with olive oil for 8-12 weeks duration in Balb/c or C57BL/6 mice, purchased from Charles River Laboratories (Wilmington, Mass.). This method is a very well validated approach that simulates the fibrogenic/fibrolysis process in humans, including, but not limited to, increased collagen in the liver and increased numbers of activated hepatic stellate cells, myofibroblasts or other fibrogenic mesenchymal cells. [See, e.g., Proctor E. and Chatamra, K. (1982) 83:1183-1190; Zhou, X. et al. (2004) 126:1795-1808; Iredale, J. P. et al. (1998) J. Clin Invest 102:538-549].

DCs Augmentation Using Flt3L Dc Expansion

For DC expansion, two approaches were used. In the first approach, hydrodynamic gene delivery of Flt3L expression plasmid was used. The Flt3L expression plasmid was a gift from Dr. Jack Wands at Brown University (Providence, R.I.). This approach has been described in detail in He, Y., et al., (2000) Hum Gene Ther 11:547-54. Briefly, the plasmid pNGVL-hFLex was obtained from the National Gene Vector Laboratory (University of Michigan, Ann Arbor, Mich.). Ten (10) μg of the pNGVL-hFLex plasmid DNA diluted in 10 ml of sterilized 0.9% NaCl solution was injected into mice through their tail vein over 10 seconds, using a 27½-gauge needle. In experiments conducted in CCL4 mice, 10 μg of the pNGVL-hFLex plasmid DNA diluted in 10 ml of sterilized 0.9% NaCl solution was injected into the CCL4 mice through their tail vein over 10 seconds, using a 27½-gauge needle one time before the last injection of CCL4. To confirm successful expansion of DCs using this method, splenocytes were isolated 10-14 days after injection of the Flt3L plasmid, stained with a fluorescent antibody specific for DEC-205, which is expressed on DCs, and analyzed by immunofluorescence to determine the percentage of DCs present in the spleen.

In the second approach, placement of melanoma cells that permanently express and secrete Flt3L was used to deliver a continuous source of systemic Flt3L. This method provides an inexpensive and rapid method of augmentation of all DC populations (classical and plasmacytoid DCs) [Gregory, S. H., et al., (2001) Cytokine 13:202-8]. The method is adapted from the method described in detail in Mach et al. (2000) Cancer Research. 60:3239-3246. In this method, B16-F10 melanoma cells are retrovirally transduced with a recombinant retrovirus engineered to have the gene for Flt3L. For the generation of the recombinant retrovirus, total RNA was obtained from C57B16 spleens using TRIzol® Reagent (Life Technologies, Inc., Grand Island, N.Y.) according to the manufacturer's instructions. Next, cDNA was synthesized using oligo-dT primers and MMLV reverse transcriptase (Life Technologies, Inc.). A PCR was performed to obtain cDNA encoding murine Flt3L. The primers used were: sense strand 5′ CATATCATGACAGTGCTGGCGCCAGCC (SEQ ID NO: 25) and antisense strand 5′ GTAAGGATCCTAGGGATGGGAGGGGAGG (SEQ ID NO: 26), derived from the published sequence [see, Lyman S. D., et al. (1993) Cell. 75:1157-1167]. The sense strand primer incorporates a BspHI restriction site upstream of the initiator ATG, and the antisense primer incorporates a BamHI restriction site downstream of the termination codon. The conditions of the PCR were: 30 cycles of 96° C. for 30 s, 50° C. for 50 s, and 72° C. for 3 min. The 711-bp amplified fragment was sequenced to confirm the integrity of the cDNA, digested with BspHI and BamHI, and subcloned into pMFG, as described previously [Dranoff G., et al. (1993). Proc. Natl. Acad. Sci. USA. 90:3539-3543]. The pMFG vector uses the MMLV long terminal repeat sequences to generate both a full-length viral RNA (for encapsidation into viral particles) and a subgenomic RNA that is responsible for expression of inserted sequences.

Following preparation of the retroviral vectors, the B16-F10 melanoma cells (a gift from Dr. Stephen Gregory (Department of Medicine, Brown University)) were prepared. B16-F10 melanoma cells (syngeneic to C57B16 mice) were maintained in DMEM containing 10% (vol/vol) FCS and penicillin/streptomycin. B16 cells were infected in the presence of polybrene (Sigma Chemical Co., St. Louis, Mo.), and unselected populations were used for study, as described previously [Dranoff G., et al. (1993). Proc. Natl. Acad. Sci. USA. 90:3539-3543]. The proportion of tumor cells transduced with the retroviral vector (which contains no selectable marker) was determined by Southern analysis. By 14 days after injection, a nearly 100-fold increase in DC numbers is observed. This method elicits comparable effects on hematopoietic populations as the injections of recombinant Flt3L protein [Maraskovsky E., et al. (1996) J. Exp. Med. 184:1953-1961].

### Isolation of DCs and In Vitro Culture

For in vitro studies of DCs and for experiments in which DCs were transferred to recipient mice (see, e.g. Example 4), DCs were isolated from Flt3L-treated donor mice. Balb/C or C57BL/6 donor mice (Charles River Laboratories) underwent DCs augmentation using Flt3L expressing plasmid administration or were transduced with melanoma cells expressing Flt3L. Donor mice were sacrificed and livers and spleens were harvested 10-14 days after the induction of augmentation. Livers or spleens from individual mice were homogenized to form a cell suspension. Lymphocytes were separated from the cell suspensions using a Percoll® (Sigma-Aldrich) density gradient. Next, the hepatocytes or splenocytes were depleted of NK, T, and B cells by magnetic negative cell selection. Specifically, the cells were labeled with biotinylated anti-NK1.1, anti-CD 19, and anti-CD3 antibodies (eBioscience™, San Diego, Calif.) each used at a concentration of 1 to 200, followed by incubation with MACS® streptavidin-coated beads (Miltenyi Biotec, Inc., Auburn, Calif.), as per the manufacturer's instructions. DCs were then positively selected from the NK, T, and B cell-depleted lymphocytes cell suspension using MACS® CD11c Microbeads magnetic cell sorting kit (Miltenyi Biotec, Inc.) according to the manufacturer's instructions. Briefly, the cells were incubated in cell labeling buffer (PBS+2 mM EDTA) and mouse IgG (diluted 1:5) to block nonspecific binding of the CD11c Microbeads. Next, cells were incubated for 15 minutes on ice with the CD11c Microbeads. Next, the cells were washed three times with 10 times volume of labeling buffer, resuspended in separation buffer (PBS+2 mM EDTA+0.5% bovine serum albumin) and then positively selected on a magnetic column.

The purified DCs were used for DCs transfer or for the assessment of MMP production in vitro. For the in vitro experiments, the DCs were cultured in 12-well low adherence culture plates (Fisher Scientific, Pittsburgh, Pa.) at a concentration of 10⁷/well in HEPES-buffered medium containing 10% fetal calf serum with or without 100 ng/ml lipopolysaccharide (LPS) isolated from E. coli (Sigma-Aldrich). 24-48 hours later the cultured DCs were harvested and used for protein and total mRNA extraction.

DC Depletion in CD11c-DTR Transgenic Mice

For DC depletion, B6.FVB-Tg(Itgax-DTR/EGFP)57Lan/J (CD11c-DTR transgenic mice, The Jackson Laboratory, Bar Harbor, Me.) were given one intravenous dose of DT (4 ng/g) by tail vein injection. Following administration of DT, DC depletion was confirmed by flow cytometry in DT-treated or control mice. 12, 36, or 60 hours after DT injection, the livers were isolated from the treated or control mice and homogenized to form a cell suspension. Hepatocytes were isolated using a Percoll® density gradient, incubated with Fc block (eBioscience) for 15-30 min at 4° C. and stained with PE-conjugated anti-CD11c and APC-conjugated anti-MHC class II antibodies (eBiosciences) for 30 minutes in the dark. The excess antibody was washed with PBS and the purity of the CD11c+ cells were analyzed by flow cytometry.

### DCs Transfer to CCL4 Mice

Expanded DCs isolated from Flt3L-treated donor mice were transferred to recipient mice that were treated with CCL4 to induce liver fibrosis. The DCs were purified from the spleens of donor mice using CD11c magnetic beads and NK/B/T cell depletion, as described above (See “Isolation of DCs”). The purity of the isolated DCs was assessed by flow cytometry and was higher than 99%. 10⁶ isolated DCs were incubated with Fc block (eBioscience) for 15-30 min at 4° C. The cells were stained with biotinylated-NK1.1, CD11c-PE-Cy7, CD45-APC-Cy7, MHCII-APC (eBioscience) for 30 in the dark. The samples were washed with PBS and stained with streptavidin-PE (eBioscience) for 30 min. The excess antibody was washed with PBS and the purity of the CD11c+ cells were analyzed by flow cytometry. One group of mice received 20×10⁶ isolated DCs (high dose-HD DCs), the second group received 5×10⁶ DCs (low dose-LD DCs) and the third group received only saline injection as a control. Purified DCs were resuspended in 200 μl cold sterile HBSS (Fisher) and administered I.V. in the tail vein. The amount of fibrosis and stellate cell activation was assessed 4 days after the DCs transfer.

### Analysis of Protein Expression by Western Blot

Purified DCs or liver samples were used for protein extraction and the extracted protein was analyzed for MMP-9 expression by Western blot analysis. Proteins were collected with Roche complete lysis M supplemented with Roche complete protease inhibitor cocktail (Roche, Nutley, N.J.) Pre-cast 10% NuPAGE® Bis-Tris gels (Invitrogen Corporation, Carlsbad, Calif.) were used to run protein at 200 volts for 1 hour, and were then transferred on PVDF membranes for 2 hours at 200 mAmp with Bio-Rad transfer system (Hercules, Calif.). The membranes were blocked in PBS+0.1% Tween 20 (PBST)+1% BSA for 1 hour at room temperature, and then incubated with a rabbit anti-MMP-9 antibody (Chemicon., Pittsburgh, Pa.) diluted 1:2,000 overnight at 4° C. with gentle rotation. The blots were then washed 3 times 15 mins each with PBST at room temperature on a shaker. The blots were then incubated with ECL™ substrate anti-rabbit IgG, horseradish peroxidase linked secondary antibody (GE Healthcare) diluted 1:5,000 in PBST-1% BSA for 1 hour at room temperature. Membranes were washed 3 times 15 mins each in PBST. Membranes were developed with Chemiluminescent HRP Substrate (Millipore, Temecula, Calif.), using Blue Basic Autorad film (ISC BioExpress, Kaysville, Utah).

Analysis of mRNA Gene Expression by qPCR

The RNA was isolated from whole liver using the RNeasy Mini Kit (Qiagen, Germantown, Md.). The cDNAs were synthesized using Sprint™ RT complete products (Clontech, Mountain View, Calif.) in a 20 μl reaction from total RNA extracted from the liver by incubating at 42° C. for 60 min and terminating at 70° C. for 10 min. The transcription level of MMPs and TIMPs was tested by quantitative PCR (qPCR) on the LightCycler™ 480 real-time PCR system (Roche) using the FastStart SYBR Green Master(Rox) (Roche) in a 10 μl reaction volume with a 45 cycle amplification. All samples were analyzed in triplicate and used only when less than a 5 cycle difference was found. Every primer set was diluted to 25 μM and the results were normalized to GAPDH.

Primer sequences used in the analyses were as follows:

| | |
|---|---|
| MMP-13 Reverse: | |
| GGCTCTGAATGGTTATGACATTCTG | (SEQ ID NO: 27) |
| MMP-13 Forward: | |
| AGAGGGTCTTCCCCGTGTTCT, | (SEQ ID NO: 28) |
| MMP-10 Reverse: | |
| CGTGCTGACTGAATCAAAGGAC, | (SEQ ID NO: 29) |
| MMP-10 Forward: | |
| CCTGTGTTGTCTGTCTCTCCAAGA, | (SEQ ID NO: 30) |
| MMP-9 Reverse: | |
| GGAAAGTCACACGCCAGAAGA, | (SEQ ID NO: 31) |
| MMP-9 Forward: | |
| AGCGTCATTCGCGTGGATA, | (SEQ ID NO: 32) |
| TIMP-1 Reverse: | |
| CTGAGAGTACGCCAGGGAAC, | (SEQ ID NO: 33) |
| TIMP-1 Forward: | |
| CATGGAAAGCCTCTGTGGAT, | (SEQ ID NO: 34) |
| TIMP-2 Reverse: | |
| CTCAGAGTACGCCAGGGAAC, | (SEQ ID NO: 35) |
| TIMP-2 Forward: | |
| GTCCATCCAGAGGCACTCAT, | (SEQ ID NO: 36)) |
| MMP-14 Reverse: | |
| CAATGGGCATTGGGTATCC, | (SEQ ID NO: 37) |
| MMP-14 Forward: | |
| AACTTTGACACCGTGGCCA, | (SEQ ID NO: 38) |
| MMP-2 Reverse: | |
| AAGGACCGGTTTATTTGGCG | (SEQ ID NO: 39) |
| MMP-2 Forward: | |
| CTCCCCCGATGCTGATACTG | (SEQ ID NO: 40) |
| GAPDH Reverse: | |
| GATCTCGCTCCTGGAAGATG | (SEQ ID NO: 41) |
| GAPDH Forward: | |
| CAATGACCCCTTCATTGACC | (SEQ ID NO: 42) |

wherein, “Forward” indicates the primer used for amplifying the desired region of RNA from the 5′ end of the region and “Reverse” indicates the primer for amplifying the desired region of RNA from the 3′ end of the region.
MMP-9 staining

For MMP-9 immunofluorescence staining and co-staining with CD11c, slides were fixed in cold acetone and rehydrated in PBS-T and then blocked in PBS with 1% BSA for 1 hour. The MMP-9 primary antibody (Santa Cruz Biotechnology® Inc., Santa Cruz, Calif.) was diluted 1:25, added to the slides and incubated 1 hour at room temperature. Slides were washed 3× in PBS and incubated with the Alexa Fluor® 488 donkey anti-goat secondary antibody (Invitrogen™ Corporation) diluted 1:400 and incubated at room temperature for 1 hour. The slides were washed 3× in PBS and incubated with anti-CD11c antibody (eBioscience) diluted 1:10 for 1 hour at room temperature. The slides were washed 3× in PBS and incubated with Alexa Fluor® 568 goat anti-hamster secondary antibody (Invitrogen) diluted 1:400. Slides were washed 3× with PBS and mounted with ProLong Gold antifade reagent with DAPI (Invitrogen). Images were taken with Zeiss Axiophot 2 microscope (Carl Zeiss Ltd, United Kingdom).

### Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Liver DCs are an Important Source of MMPs

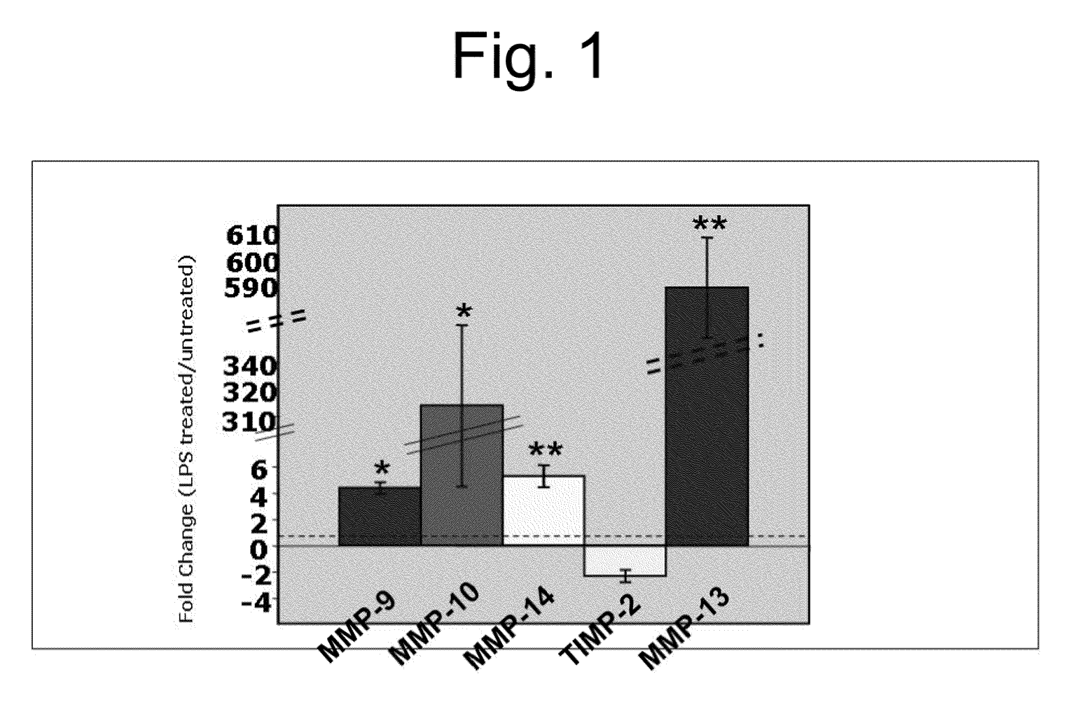

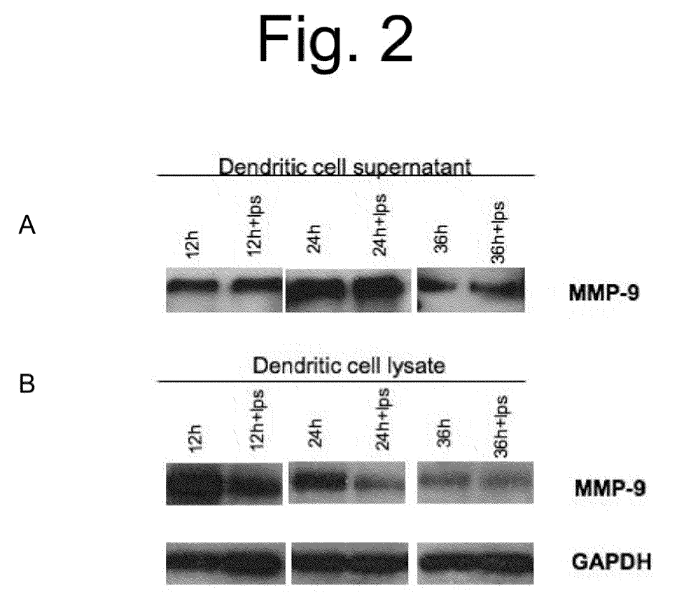

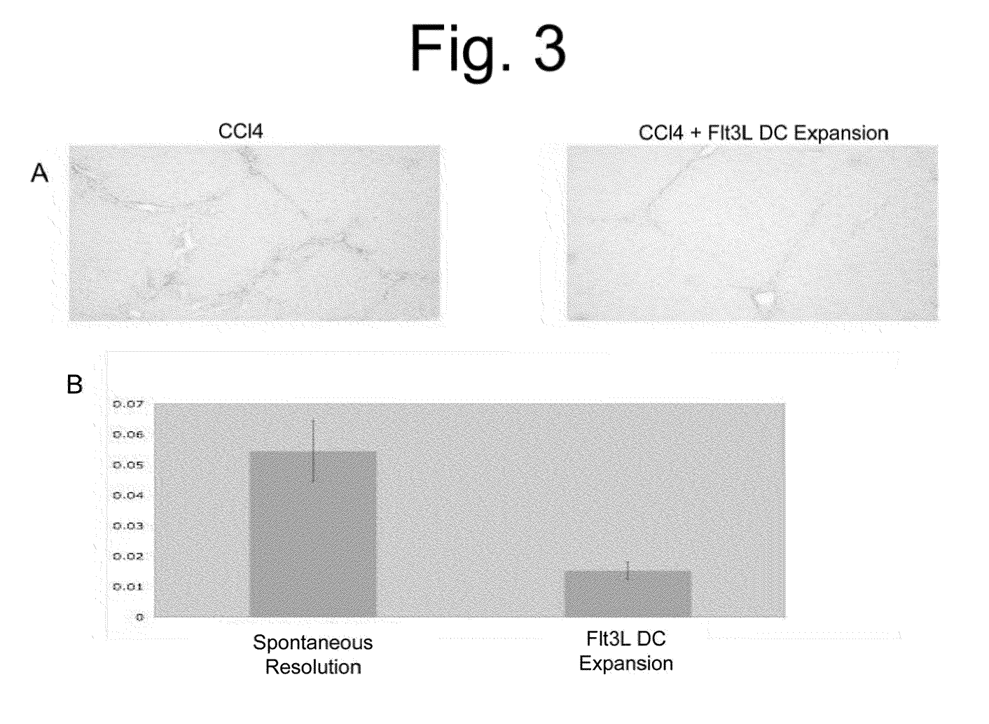

In order to determine whether DCs might play an important role in fibrosis regression, potentially through the production of MMPs, the mRNA expression levels of MMPs in Flt3L-expanded DCs were determined following culture in the presence or absence of LPS, as described above. Following LPS stimulation of Flt3L-expanded DCs, mRNA expression levels of MMP-9, MMP-10, MMP-13, and MMP-14 were upregulated (FIG. 1). Furthermore, the mRNA expression level of TIMP-2 was decreased during DC activation (FIG. 1). The data were normalized to the expression of the housekeeping gene GAPDH. Because MMP-9 has been previously shown to be involved in the migration of DCs, the expression levels of MMP-9 protein in the culture supernatants and in DC cell lysates were evaluated by Western blot analysis. Following LPS stimulation, MMP-9 protein levels decreased in the DC cell lysates over time (FIG. 2B) and increased in the culture supernatant over the same time (FIG. 2A), strongly suggesting an active secretion of MMP-9 during LPS-induced DCs maturation.

### Example 2

### Flt3L DC Expansion Increases Fibrosis Regression and is Associated with Increased MMP-9 Expression in the Liver

Liver fibrosis was induced by administering CCL4 to mice for 8 weeks. Following treatment with CCL4, CCL4-treated mice (CCL4 mice) exhibit the hallmarks of hepatic fibrosis. After the last dose of CCL4, DCs were augmented using Flt3L melanoma cells placed subcutaneously in the CCL4 mice or by injecting the Flt3L expressing. The effect of DC augmentation by Flt3L (CCL4+Flt3L DC Expansion) on fibrosis regression was assessed by staining of collagen in the liver with Sirius Red (Sigma-Aldrich) and compared to Sirius Red staining of liver sections from CCL4 mice that did not receive Flt3L (CCL4) (FIG. 3A). Three (3) days after the last CCL4 dose there was significantly less collagen in the liver sections from the group that had been treated with Flt3L (Flt3L DC Expansion) compared to the group that was not treated with Flt3L (Spontaneous Resolution), confirming that there was increased fibrosis resolution in the Flt3L-treated group (FIG. 3B).

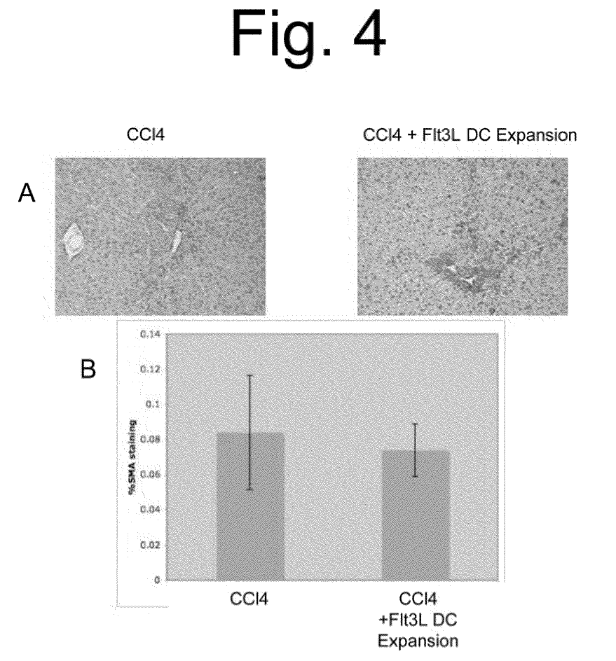

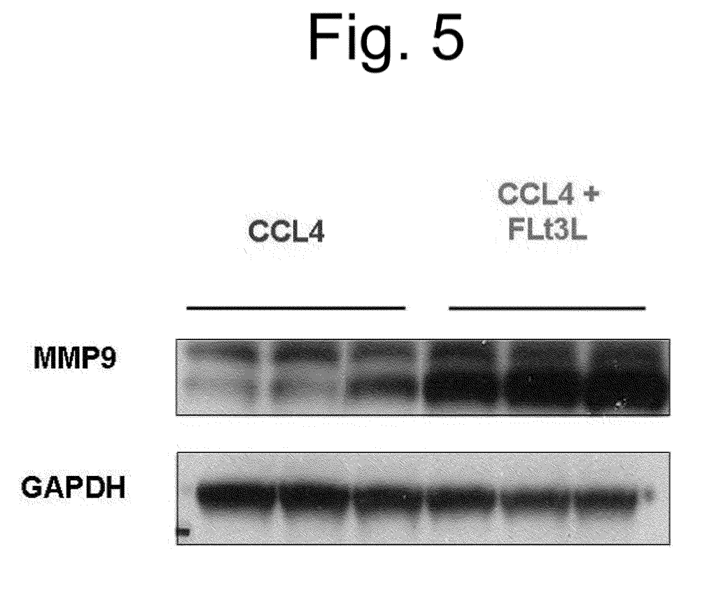

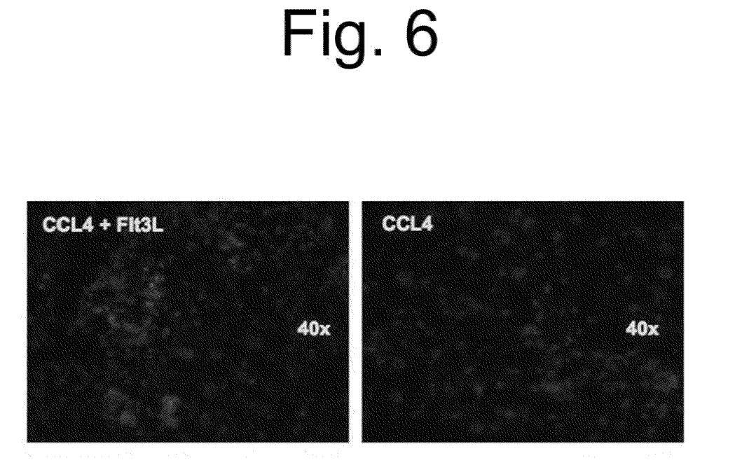

To investigate the mechanism underlying this observation, the level of stellate cell activation was evaluated by staining for α-smooth muscle actin in CCL4 mice (CCL4) and in CCL4 mice that received Flt3L to expand DCs (CCL4+Flt3L DC Expansion) (FIG. 4A). Although there was a trend toward a low level of activation of stellate cells in the expanded group, as indicated by the graph in FIG. 4B, this trend did not reach statistical significance. However, at this early time point during fibrosis regression, the Flt3L-induced expansion of DCs (CCL4+Flt3L) was associated with increased expression of MMP-9 protein compared to the non-expanded group (CCL4), as determined by Western blot and immunofluorescence (FIGS. 5 and 6, respectively). For Western blot analysis, GAPDH was used as a loading control.

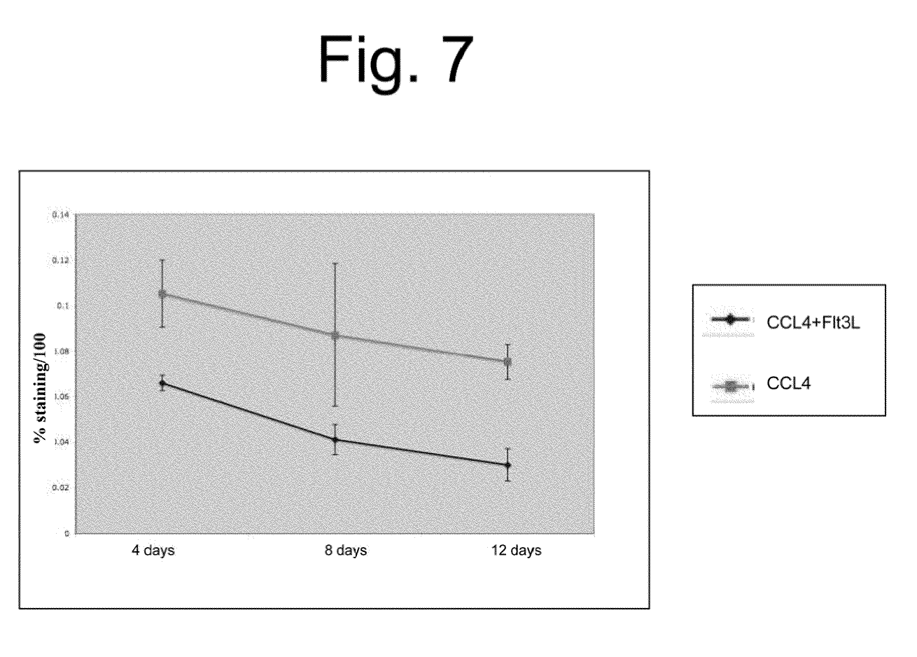

To determine whether the same effect was present in long-term-induced fibrosis (i.e., “mature” collagen) the same experiments were repeated after 15 weeks of CCL4 administration. The same effect on fibrosis resolution was observed, as determined by Sirius Red staining of collagen 4, 8 and 12 days after CCL4 discontinuation (FIG. 7), indicating that Flt3L-induced fibrosis regression occurs in mice having disease characteristics that mimic acute fibrosis and in mice having disease characteristics that mimic chronic fibrosis. In FIG. 7, the results are expressed as a percent of total area within the field of view (e.g., a value of 0.1 indicates that 10% of the cells in the microscopic field (at 10× power) stained positive for collagen with Sirius Red.

### Example 3

### DC Depletion Slows Down Fibrosis Regression

Using CD11c-DTR transgenic mice, it was determined whether depletion of DCs is associated with a decreased rate of fibrosis resolution. Following administration of DT, DC depletion was confirmed by flow cytometry in DT-treated or control mice. 12, 36, or 60 hours after DT injection, the livers were isolated from the treated or control mice and stained with anti-CD11c and anti-MHC class II antibodies. As shown in FIG. 12, the number of MHC class II high, CD11c+DCs in the liver was significantly reduced 12 hours after administration of DT (FIG. 12). Moreover, administration of DT was followed 24-36 hours later by the depletion of MHC class II-high, CD11c+DCs at levels 20-30% from baseline.

Fibrosis was induced in the CD11c-DTR transgenic mice using the above-described CCL4-induced model of fibrosis. After 12 weeks of administration of CCL4, the mice were separated into two groups: one group received one dose of DT (4 ng/g) (DC Depleted) one day after the last CCL4 dose and the second group received only saline solution (Non-depleted). The effect of DC depletion on fibrosis regression was assessed 4 days after the last CCL4 dose (3 days after the DT dose).

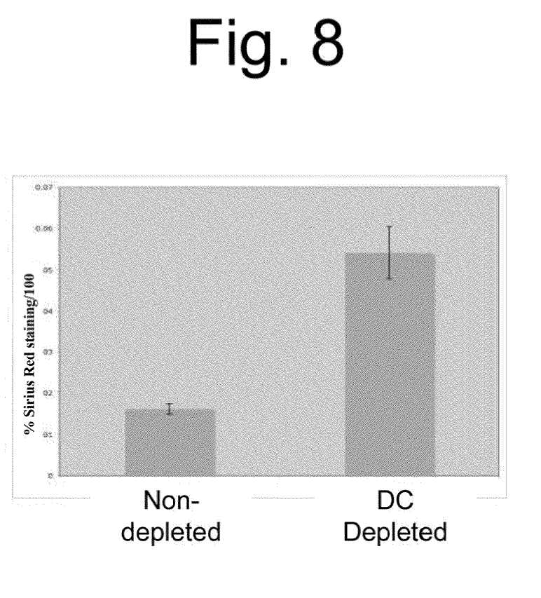

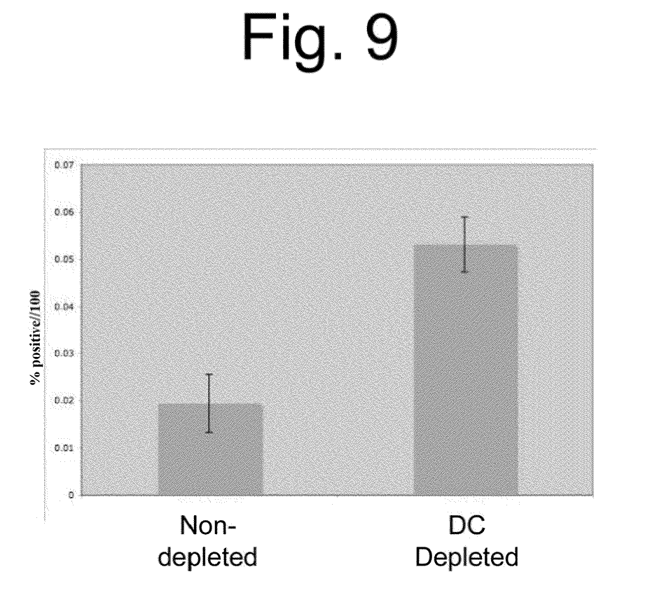

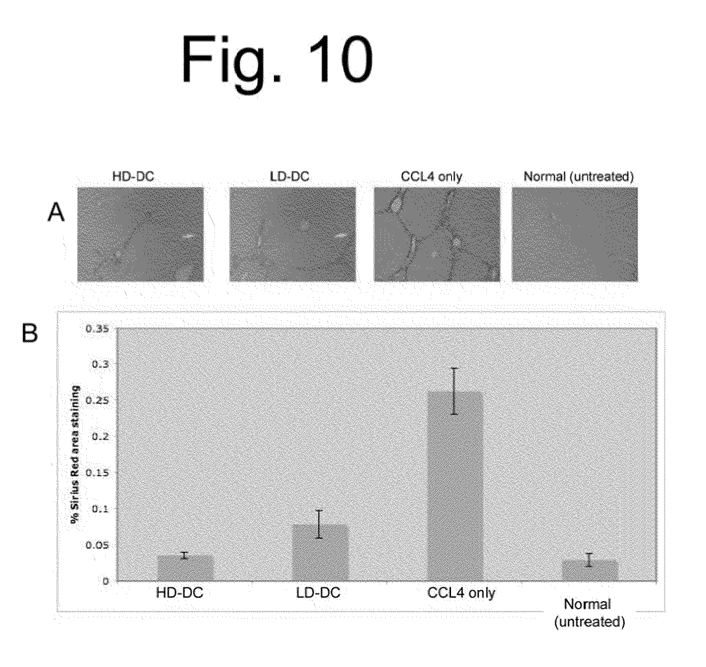

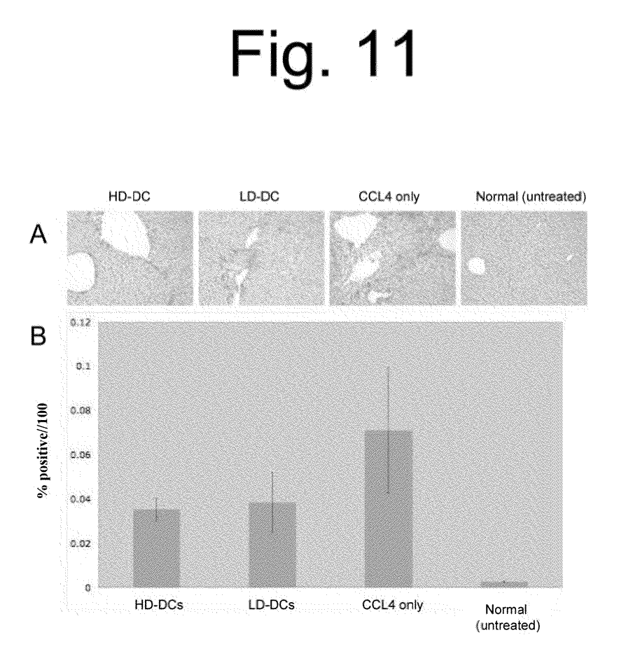

Sirius Red staining for collagen of liver sections from treated mice showed significantly less fibrosis in non-depleted mice compared to DT-treated, DC-depleted mice (FIG. 8). Moreover, there were significantly more activated stellate cells in DC-depleted mice, as measured by α-smooth muscle actin staining, further demonstrating the importance of classical DCs in clearance of activated stellate cells after discontinuation of CCL4 (FIG. 9).

### Example 4

### Accelerated Fibrosis Regression after Flt3L-Induced DCs Expansion is the Result of a Specific DC Effect

Because the FLt3L treatment is associated not only with DC expansion but also with other non-DC populations, including NK and NKT cells, in the next set of experiments it was determined whether specific transfer of DCs in Balb/c mice with CCL4-induced liver fibrosis would simulate the same effect. Recipient mice were treated with CCL4 for 15 weeks to induce long-term fibrosis. In the donor mice, DCs were expanded and purified from the spleen using CD11c magnetic beads and NK/B/T cell depletion, as described above. The purity of the purified DCs was assessed by flow cytometry and was greater than 99%. The mice were then split into 3 groups 2 days after the last CCL4 dose. One group received 20×10⁶ DCs (high dose (HD)-DCs), the second group received 5×10⁶ DCs (low dose-(LD)-DC) and the third group received only saline injection (CCL4 only). The amount of fibrosis and stellate cell activation was assessed 4 days after the DCs transfer.

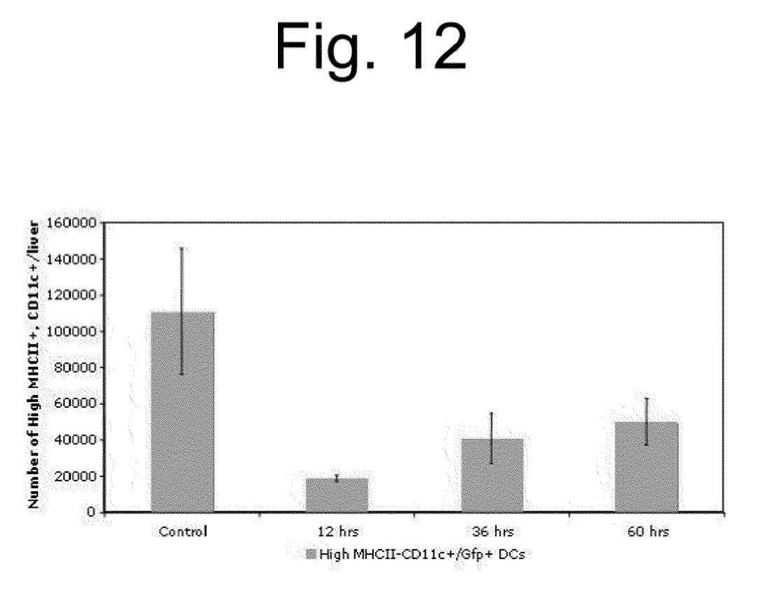

It was found that DC transfer accelerated fibrosis regression in both HD-DC and LD-DC groups compared with the CCL4 only group (spontaneous resolution). Sirius Red staining of liver sections (images shown in FIG. 10A) from HD-DC and LD-DC groups had decreased levels of collagen compared to the CCL4 only group (quantitation shown in FIG. 10B). Shown as a control, normal (untreated) mice had very low levels of collagen. (FIG. 10B) HD-DC and LD-DC transfer was also associated with low levels of α-smooth muscle actin staining compared to the CCL4 only group (FIGS. 11A and 11B). These experiments demonstrate that there is a direct role for DCs in fibrosis resolution in Flt3L-treated mice.

### Summary

The present invention demonstrates that Flt3L-induced augmentation of DCs accelerates liver fibrosis regression in the CCL4 induced model of liver fibrosis. The effect is associated at early time points during resolution with increased MMP-9 levels. Based on the effect of DC transfer experiments, the accelerated fibrosis resolution is the specific result of Flt3L-induced augmentation of DCs. Furthermore, depletion of classical DCs in CD11c-DTR transgenic mice is associated with a persistent activation of stellate cells and a slow fibrosis resolution, confirming the importance of DCs for fibrosis resolution. Therefore, Flt3L treatment and DC augmentation may provide a new approach for accelerating fibrosis regression in humans.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

While the compositions and methods of this invention have been described in terms of specific embodiments, it will be apparent to those of skill in the art that variations may be applied to the compositions and methods and in the steps or in the sequence of steps of the method described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope of the invention as defined by the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

### SEQUENCES

### Methods and Compositions for Treatment of Fibrosis

### Inventors

### Costica Aloman, Scott Friedman, Miriam Merad

### ALL PRIMER SEQUENCES (SEQ ID NO: 25-42) ARE FOR AMPLIFYING MURINE GENES:

| SEQ ID NO: 1 | |
|---|---|
| NP 004110, Human Flt3 ligand | |
| 1 | mpalardggq lpllvvfsam ifgtitnqdl pvikcvlinh knndssvgks ssypmvsesp |
| 61 | edlgcalrpq ssgtvyeaaa vevdvsasit lqvivdapgn isclwvfkhs slncqphfdl |
| 121 | qnrgvvsmvi lkmtetqage yllfiqseat nytilftvsi rntllytlrr pyfrkmenqd |
| 181 | alvcisesvp epivewvlcd sqgesckees pavvkkeekv lhelfgtdir ccarnelgre |
| 241 | ctrlftidln qtpqttlpql flkvgeplwi rckavhvnhg fgltwelenk aleegnyfam |
| 301 | stystnrtmi rilfafvssv arndtgyytc ssskhpsqsa lvtivekgfi natnssedye |
| 361 | idqyeefcfs vrfkaypqir ctwtfsrksf pceqkgldng ysiskfcnhk hqpgeyifha |
| 421 | enddaqftkm ftlnirrkpq vlaeasasqa scfsdgyplp swtwkkcsdk spncteeite |
| 481 | gvwnrkanrk vfgqwvssst lnmseaikgf lvkccaynsl gtscetilln spgpfpfiqd |
| 541 | nisfyatigv cllfivvltl lichkykkqf ryesqlqmvq vtgssdneyf yvdfreyeyd |
| 601 | lkwefprenl efgkvlgsga fgkvmnatay gisktgvsiq vavkmlkeka dsserealms |
| 661 | elkmmtqlgs henivnllga ctlsgpiyli feyccygdll nylrskrekf hrtwteifke |
| 721 | hnfsfyptfq shpnssmpgs revqihpdsd qisglhgnsf hsedeieyen qkrleeeedl |
| 781 | nvltfedllc fayqvakgme flefkscvhr dlaarnvlvt hgkvvkicdf glardimsds |
| 841 | nyvvrgnarl pvkwmapesl fegiytiksd vwsygillwe ifslgvnpyp gipvdanfyk |
| 901 | liqngfkmdq pfyateeiyi imqscqafds rkrpsfpnlt sflgcqlada eeamyqnvdg |
| 961 | rvsecphtyq nrrpfsremd lgllspqaqv eds |
| SEQ ID NO: 2 | |
| NM 004119, Human Flt3 ligand | |
| 1 | acctgcagcg cgaggcgcgc cgctccaggc ggcatcgcag ggctgggccg gcgcggcctg |
| 61 | gggaccccgg gctccggagg ccatgccggc gttggcgcgc gacggcggcc agctgccgct |
| 121 | gctcgttgtt ttttctgcaa tgatatttgg gactattaca aatcaagatc tgcctgtgat |
| 181 | caagtgtgtt ttaatcaatc ataagaacaa tgattcatca gtggggaagt catcatcata |
| 241 | tcccatggta tcagaatccc cggaagacct cgggtgtgcg ttgagacccc agagctcagg |
| 301 | gacagtgtac gaagctgccg ctgtggaagt ggatgtatct gcttccatca cactgcaagt |
| 361 | gctggtcgac gccccaggga acatttcctg tctctgggtc tttaagcaca gctccctgaa |
| 421 | ttgccagcca cattttgatt tacaaaacag aggagttgtt tccatggtca ttttgaaaat |
| 481 | gacagaaacc caagctggag aatacctact ttttattcag agtgaagcta ccaattacac |
| 541 | aatattgttt acagtgagta taagaaatac cctgctttac acattaagaa gaccttactt |
| 601 | tagaaaaatg gaaaaccagg acgccctggt ctgcatatct gagagcgttc cagagccgat |
| 661 | cgtggaatgg gtgctttgcg attcacaggg ggaaagctgt aaagaagaaa gtccagctgt |
| 721 | tgttaaaaag gaggaaaaag tgcttcatga attatttggg acggacataa ggtgctgtgc |
| 781 | cagaaatgaa ctgggcaggg aatgcaccag gctgttcaca atagatctaa atcaaactcc |
| 841 | tcagaccaca ttgccacaat tatttcttaa agtaggggaa cccttatgga taaggtgcaa |
| 901 | agctgttcat gtgaaccatg gattcgggct cacctgggaa ttagaaaaca aagcactcga |
| 961 | ggagggcaac tactttgaga tgagtaccta ttcaacaaac agaactatga tacggattct |
| 1021 | gtttgctttt gtatcatcag tggcaagaaa cgacaccgga tactacactt gttcctcttc |
| 1081 | aaagcatccc agtcaatcag ctttggttac catcgtagaa aagggattta taaatgctac |
| 1141 | caattcaagt gaagattatg aaattgacca atatgaagag ttttgttttt ctgtcaggtt |
| 1201 | taaagcctac ccacaaatca gatgtacgtg gaccttctct cgaaaatcat ttccttgtga |
| 1261 | gcaaaagggt cttgataacg gatacagcat atccaagttt tgcaatcata agcaccagcc |
| 1321 | aggagaatat atattccatg cagaaaatga tgatgcccaa tttaccaaaa tgttcacgct |
| 1381 | gaatataaga aggaaacctc aagtgctcgc agaagcatcg gcaagtcagg cgtcctgttt |
| 1441 | ctcggatgga tacccattac catcttggac ctggaagaag tgttcagaca agtctcccaa |
| 1501 | ctgcacagaa gagatcacag aaggagtctg gaatagaaag gctaacagaa aagtgtttgg |
| 1561 | acagtgggtg tcgagcagta ctctaaacat gagtgaagcc ataaaagggt tcctggtcaa |
| 1621 | gtgctgtgca tacaattccc ttggcacatc ttgtgagacg atccttttaa actctccagg |
| 1681 | ccccttccct ttcatccaag acaacatctc attctatgca acaattggtg tttgtctcct |
| 1741 | cttcattgtc gttttaaccc tgctaatttg tcacaagtac aaaaagcaat ttaggtatga |
| 1801 | aagccagcta cagatggtac aggtgaccgg ctcctcagat aatgagtact tctacgttga |
| 1861 | tttcagagaa tatgaatatg atctcaaatg ggagtttcca agagaaaatt tagagtttgg |
| 1921 | gaaggtacta ggatcaggtg cttttggaaa agtgatgaac gcaacagctt atggaattag |
| 1981 | caaaacagga gtctcaatcc aggttgccgt caaaatgctg aaagaaaaag cagacagctc |
| 2041 | tgaaagagag gcactcatgt cagaactcaa gatgatgacc cagctgggaa gccacgagaa |
| 2101 | tattgtgaac ctgctggggg cgtgcacact gtcaggacca atttacttga tttttgaata |
| 2161 | ctgttgctat ggtgatcttc tcaactatct aagaagtaaa agagaaaaat ttcacaggac |
| 2221 | ttggacagag attttcaagg aacacaattt cagtttttac cccactttcc aatcacatcc |
| 2281 | aaattccagc atgcctggtt caagagaagt tcagatacac ccggactcgg atcaaatctc |
| 2341 | agggcttcat gggaattcat ttcactctga agatgaaatt gaatatgaaa accaaaaaag |
| 2401 | gctggaagaa gaggaggact tgaatgtgct tacatttgaa gatcttcttt gctttgcata |
| 2461 | tcaagttgcc aaaggaatgg aatttctgga atttaagtcg tgtgttcaca gagacctggc |
| 2521 | cgccaggaac gtgcttgtca cccacgggaa agtggtgaag atatgtgact ttggattggc |
| 2581 | tcgagatatc atgagtgatt ccaactatgt tgtcaggggc aatgcccgtc tgcctgtaaa |
| 2641 | atggatggcc cccgaaagcc tgtttgaagg catctacacc attaagagtg atgtctggtc |
| 2701 | atatggaata ttactgtggg aaatcttctc acttggtgtg aatccttacc ctggcattcc |
| 2761 | ggttgatgct aacttctaca aactgattca aaatggattt aaaatggatc agccatttta |
| 2821 | tgctacagaa gaaatataca ttataatgca atcctgctgg gcttttgact caaggaaacg |
| 2881 | gccatccttc cctaatttga cttcgttttt aggatgtcag ctggcagatg cagaagaagc |
| 2941 | gatgtatcag aatgtggatg gccgtgtttc ggaatgtcct cacacctacc aaaacaggcg |
| 3001 | acctttcagc agagagatgg atttggggct actctctccg caggctcagg tcgaagattc |
| 3061 | gtagaggaac aatttagttt taaggacttc atccctccac ctatccctaa caggctgtag |
| 3121 | attaccaaaa caagattaat ttcatcacta aaagaaaatc tattatcaac tgctgcttca |
| 3181 | ccagactttt ctctagaagc tgtctgcgtt tactcttgtt ttcaaaggga cttttgtaaa |
| 3241 | atcaaatcat cctgtcacaa ggcaggagga gctgataatg aactttattg gagcattgat |
| 3301 | ctgcatccaa ggccttctca ggctggcttg agtgaattgt gtacctgaag tacagtatat |
| 3361 | tcttgtaaat acataaaaca aaagcatttt gctaaggaga agctaatatg attttttaag |
| 3421 | tctatgtttt aaaataatat gtaaattttt cagctattta gtgatatatt ttatgggtgg |
| 3481 | gaataaaatt tctactacag aattgcccat tattgaatta tttacatggt ataattaggg |
| 3541 | caagtcttaa ctggagttca cgaaccccct gaaattgtgc acccatagcc acctacacat |
| 3601 | tccttccaga gcacgtgtgc ttttacccca agatacaagg aatgtgtagg cagctatggt |
| 3661 | tgtcacagcc taagatttct gcaacaacag gggttgtatt gggggaagtt tataatgaat |
| 3721 | aggtgttcta ccataaagag taatacatca cctagacact ttggcggcct tcccagactc |
| 3781 | agggccagtc agaagtaaca tggaggatta gtattttcaa taaagttact cttgtcccca |
| 3841 | caaaaaaa |
| SEQ ID NO: 3 | |
| NP 034359, Murine Flt3 ligand | |
| 1 | mralaqrsdr rllllvvlsv miletvtnqd lpvikcvlis henngssagk pssyrmvrgs |
| 61 | pedlqcaprr qsegtvyeaa tvevaesgsi tlqvqlatpg dlsclwvfkh sslgcqphfd |
| 121 | lqnrgivsma ilnvtetqag eyllhiqsea anytvlftvn vrdtqlyvlr rpyfrkmenq |
| 181 | dallcisegv peptvewvlc sshresckee gpavvrkeek vlhelfgtdi rccarnalgr |
| 241 | ectklftidl nqapqstlpq lflkvgeplw irckaihvnh gfgltweled kaleegayfe |
| 301 | mstystnrtm irillafvss vgrndtgyyt cssskhpsqs alvtilekgf inatssqeey |
| 361 | eidpyekfcf svrfkaypri rctwifsqas fpceqrgled gysiskfcdh knkpgeyify |
| 421 | aenddaqftk mftlnirkkp qvlanasasq ascssdgypl pswtwkkcsd kspncteeip |
| 481 | egvwnkkanr kvfgqwvsss tlnmseagkg llvkccayns mgtscetifl nspgpfpfiq |
| 541 | dnisfyatig lclpfivvli vlichkykkq fryesqlqmi qvtgpldney fyvdfrdyey |
| 601 | dlkwefpren lefgkvlgsg afgrvmnata ygisktgvsi qvavkmlkek adscekealm |
| 661 | selkmmthlg hhdnivnllg actlsgpvyl ifeyccygdl lnylrskrek fhrtwteifk |
| 721 | ehnfsfyptf qahsnssmpg srevqlhppl dqlsgfngnl ihsedeieye nqkrlaeeee |
| 781 | edlnvltfed llcfayqvak gmeflefksc vhrdlaarnv lvthgkvvki cdfglardil |
| 841 | sdssyvvrgn arlpvkwmap eslfegiyti ksdvwsygil lweifslgvn pypgipvdan |
| 901 | fykliqsgfk meqpfyateg iyfvmqscwa fdsrkrpsfp nltsflgcql aeaeeamyqn |
| 961 | mggnvpehps iyqnrrplsr eagseppspq aqvkihgers |
| SEQ ID NO: 4 | |
| NM 010229, Murine Flt3 ligand | |
| 1 | gggcacgtgg gatcggctgc agcactgcgc cagttcagcc cgcctagcag cgagcggccg |
| 61 | cggcctctgg agagaggttc ctccccctct gctctgcacc agtccgaggg aatctgtggt |
| 121 | cagtgacgcg catccttcag cgagccacct gcagcccggg gcgcgccgct gggaccgcat |
| 181 | cacaggctgg gccggcggcc tggctaccgc gcgctccgga ggccatgcgg gcgttggcgc |
| 241 | agcgcagcga ccggcggctg ctgctgcttg ttgttttgtc agtaatgatt cttgagaccg |
| 301 | ttacaaacca agacctgcct gtgatcaagt gtgttttaat cagtcatgag aacaatggct |
| 361 | catcagcggg aaagccatca tcgtaccgaa tggtgcgagg atccccagaa gacctccagt |
| 421 | gtgccccgag gcgccagagt gaagggacgg tatatgaagc ggccaccgtg gaggtggccg |
| 481 | agtctgggtc catcaccctg caagtgcagc tcgccacccc aggggacctt tcctgcctct |
| 541 | gggtctttaa gcacagctcc ctgggctgcc agccgcactt tgatttacaa aacagaggaa |
| 601 | tcgtttccat ggccatcttg aacgtgacag agacccaggc aggagaatac ctactccata |
| 661 | ttcagagcga agccgccaac tacacagtac tgttcacagt gaatgtaaga gatacacagc |
| 721 | tgtacgtgct aagaagacct tactttagga agatggaaaa ccaggacgca ctgctctgca |
| 781 | tctccgaggg tgttccagag cccactgtgg agtgggtgct ctgcagctcc cacagggaaa |
| 841 | gctgtaaaga agaaggccct gctgttgtca gaaaggagga aaaggtactt catgagttgt |
| 901 | tcggaacaga catcagatgc tgtgctagaa atgcactggg ccgcgaatgc accaagctgt |
| 961 | tcaccataga tctaaaccag gctcctcaga gcacactgcc ccagttattc ctgaaagtgg |
| 1021 | gggaaccctt gtggatcagg tgtaaggcca tccatgtgaa ccatggattc gggctcacct |
| 1081 | gggagctgga agacaaagcc ctggaggagg gcagctactt tgagatgagt acctactcca |
| 1141 | caaacaggac catgattcgg attctcttgg cctttgtgtc ttccgtggga aggaacgaca |
| 1201 | ccggatatta cacctgctct tcctcaaagc accccagcca gtcagcgttg gtgaccatcc |
| 1261 | tagaaaaagg gtttataaac gctaccagct cgcaagaaga gtatgaaatt gacccgtacg |
| 1321 | aaaagttctg cttctcagtc aggtttaaag cgtacccacg aatccgatgc acgtggatct |
| 1381 | tctctcaagc ctcatttcct tgtgaacaga gaggcctgga ggatgggtac agcatatcta |
| 1441 | aattttgcga tcataagaac aagccaggag agtacatatt ctatgcagaa aatgatgacg |
| 1501 | cccagttcac caaaatgttc acgctgaata taagaaagaa acctcaagtg ctagcaaatg |
| 1561 | cctcagccag ccaggcgtcc tgttcctctg atggctaccc gctaccctct tggacctgga |
| 1621 | agaagtgttc ggacaaatct cccaattgca cggaggaaat cccagaagga gtttggaata |
| 1681 | aaaaggctaa cagaaaagtg tttggccagt gggtgtcgag cagtactcta aatatgagtg |
| 1741 | aggccgggaa agggcttctg gtcaaatgct gtgcgtacaa ttctatgggc acgtcttgcg |
| 1801 | aaaccatctt tttaaactca ccaggcccct tccctttcat ccaagacaac atctccttct |
| 1861 | atgcgaccat tgggctctgt ctccccttca ttgttgttct cattgtgttg atctgccaca |
| 1921 | aatacaaaaa gcaatttagg tacgagagtc agctgcagat gatccaggtg actggccccc |
| 1981 | tggataacga gtacttctac gttgacttca gggactatga atatgacctt aagtgggagt |
| 2041 | tcccgagaga gaacttagag tttgggaagg tcctggggtc tggcgctttc gggagggtga |
| 2101 | tgaacgccac ggcctatggc attagtaaaa cgggagtctc aattcaggtg gcggtgaaga |
| 2161 | tgctaaaaga gaaagctgac agctgtgaaa aagaagctct catgtcggag ctcaaaatga |
| 2221 | tgacccacct gggacaccat gacaacatcg tgaatctgct gggggcatgc acactgtcag |
| 2281 | ggccagtgta cttgattttt gaatattgtt gctatggtga cctcctcaac tacctaagaa |
| 2341 | gtaaaagaga gaagtttcac aggacatgga cagagatttt taaggaacat aatttcagtt |
| 2401 | tttaccctac tttccaggca cattcaaatt ccagcatgcc tggttcacga gaagttcagt |
| 2461 | tacacccgcc cttggatcag ctctcagggt tcaatgggaa tttaattcat tctgaagatg |
| 2521 | agattgaata tgaaaaccag aagaggctgg cagaagaaga ggaggaagat ttgaacgtgc |
| 2581 | tgacgtttga agacctcctt tgctttgcgt accaagtggc caaaggcatg gaattcctgg |
| 2641 | agttcaagtc gtgtgtccac agagacctgg cagccaggaa tgtgttggtc acccacggga |
| 2701 | aggtggtgaa gatctgtgac tttggactgg cccgagacat cctgagcgac tccagctacg |
| 2761 | tcgtcagggg caacgcacgg ctgccggtga agtggatggc acctgagagc ttatttgaag |
| 2821 | ggatctacac aatcaagagt gacgtctggt cctacggcat ccttctctgg gagatatttt |
| 2881 | cactgggtgt gaacccttac cctggcattc ctgtcgacgc taacttctat aaactgattc |
| 2941 | agagtggatt taaaatggag cagccattct atgccacaga agggatatac tttgtaatgc |
| 3001 | aatcctgctg ggcttttgac tcaaggaagc ggccatcctt ccccaacctg acttcatttt |
| 3061 | taggatgtca gctggcagag gcagaagaag cgatgtatca gaacatgggt ggcaacgtcc |
| 3121 | cagaacatcc atccatctac caaaacaggc ggcccctcag cagagaggca ggctcagagc |
| 3181 | cgccatcgcc acaggcccag gtgaagattc acggagaaag aagttagcga ggaggccttg |
| 3241 | gaccccgcca ccctagcagg ctgtagacca cagagccaag attagcctcg cctctgagga |
| 3301 | agcgccctac aggccgttgc ttcgctggac ttttctctag atgctgtctg ccattactcc |
| 3361 | aaagtgactt ctataaaatc aaacctctcc tcgcacaggt gggagagcca ataatgagac |
| 3421 | ttgttggtga gcccgcctac cctggggggc ctttccaggc cccccaggct tgaggggaaa |
| 3481 | gccatgtatc tgaaatatag tatattcttg taaatacgtg aaacaaacca aacccgtttt |
| 3541 | ttgctaaggg aaagctaaat atgattttta aaaatctatg ttttaaaata ctatgtaact |
| 3601 | ttttcatcta tttagtgata tattttatgg atggaaataa actttctact gtagaaaaaa |
| 3661 | aaaa |
| SEQ ID NO: 5 | |
| NM 002421 Human MMP-1 | |
| 1 | gggatattgg agtagcaaga ggctgggaag ccatcactta ccttgcactg agaaagaaga |
| 61 | caaaggccag tatgcacagc tttcctccac tgctgctgct gctgttctgg ggtgtggtgt |
| 121 | ctcacagctt cccagcgact ctagaaacac aagagcaaga tgtggactta gtccagaaat |
| 181 | acctggaaaa atactacaac ctgaagaatg atgggaggca agttgaaaag cggagaaata |
| 241 | gtggcccagt ggttgaaaaa ttgaagcaaa tgcaggaatt ctttgggctg aaagtgactg |
| 301 | ggaaaccaga tgctgaaacc ctgaaggtga tgaagcagcc cagatgtgga gtgcctgatg |
| 361 | tggctcagtt tgtcctcact gaggggaacc ctcgctggga gcaaacacat ctgacctaca |
| 421 | ggattgaaaa ttacacgcca gatttgccaa gagcagatgt ggaccatgcc attgagaaag |
| 481 | ccttccaact ctggagtaat gtcacacctc tgacattcac caaggtctct gagggtcaag |
| 541 | cagacatcat gatatctttt gtcaggggag atcatcggga caactctcct tttgatggac |
| 601 | ctggaggaaa tcttgctcat gcttttcaac caggcccagg tattggaggg gatgctcatt |
| 661 | ttgatgaaga tgaaaggtgg accaacaatt tcagagagta caacttacat cgtgttgcgg |
| 721 | ctcatgaact cggccattct cttggactct cccattctac tgatatcggg gctttgatgt |
| 781 | accctagcta caccttcagt ggtgatgttc agctagctca ggatgacatt gatggcatcc |
| 841 | aagccatata tggacgttcc caaaatcctg tccagcccat cggcccacaa accccaaaag |
| 901 | cgtgtgacag taagctaacc tttgatgcta taactacgat tcggggagaa gtgatgttct |
| 961 | ttaaagacag attctacatg cgcacaaatc ccttctaccc ggaagttgag ctcaatttca |
| 1021 | tttctgtttt ctggccacaa ctgccaaatg ggcttgaagc tgcttacgaa tttgccgaca |
| 1081 | gagatgaagt ccggtttttc aaagggaata agtactgggc tgttcaggga cagaatgtgc |
| 1141 | tacacggata ccccaaggac atctacagct cctttggctt ccctagaact gtgaagcata |
| 1201 | tcgatgctgc tctttctgag gaaaacactg gaaaaaccta cttctttgtt gctaacaaat |
| 1261 | actggaggta tgatgaatat aaacgatcta tggatccagg ttatcccaaa atgatagcac |
| 1321 | atgactttcc tggaattggc cacaaagttg atgcagtttt catgaaagat ggatttttct |
| 1381 | atttctttca tggaacaaga caatacaaat ttgatcctaa aacgaagaga attttgactc |
| 1441 | tccagaaagc taatagctgg ttcaactgca ggaaaaattg aacattacta atttgaatgg |
| 1501 | aaaacacatg gtgtgagtcc aaagaaggtg ttttcctgaa gaactgtcta ttttctcagt |
| 1561 | catttttaac ctctagagtc actgatacac agaatataat cttatttata cctcagtttg |
| 1621 | catatttttt tactatttag aatgtagccc tttttgtact gatataattt agttccacaa |
| 1681 | atggtgggta caaaaagtca agtttgtggc ttatggattc atataggcca gagttgcaaa |
| 1741 | gatcttttcc agagtatgca actctgacgt tgatcccaga gagcagcttc agtgacaaac |
| 1801 | atatcctttc aagacagaaa gagacaggag acatgagtct ttgccggagg aaaagcagct |
| 1861 | caagaacaca tgtgcagtca ctggtgtcac cctggatagg caagggataa ctcttctaac |
| 1921 | acaaaataag tgttttatgt ttggaataaa gtcaaccttg tttctactgt ttt |
| SEQ ID NO: 6 | |
| NP 002412 Human MMP-1 preprotein | |
| 1 | mhsfppllll lfwgvvshsf patletqeqd vdlvqkylek yynlkndgrq vekrrnsgpv |
| 61 | veklkqmqef fglkvtgkpd aetlkvmkqp rcgvpdvaqf vltegnprwe qthltyrien |
| 121 | ytpdlpradv dhaiekafql wsnvtpltft kvsegqadim isfvrgdhrd nspfdgpggn |
| 181 | lahafqpgpg iggdahfded erwtnnfrey nlhrvaahel ghslglshst digalmypsy |
| 241 | tfsgdvqlaq ddidgiqaiy grsqnpvqpi gpqtpkacds kltfdaitti rgevmffkdr |
| 301 | fymrtnpfyp evelnfisvf wpqlpnglea ayefadrdev rffkgnkywa vqgqnvlhgy |
| 361 | pkdiyssfgf prtvkhidaa lseentgkty ffvankywry deykrsmdpg ypkmiahdfp |
| 421 | gighkvdavf mkdgffyffh gtrqykfdpk tkriltlqka nswfncrkn |
| SEQ ID NO: 7 | |
| NM 004994 Human MMP-9 | |
| 1 | agacacctct gccctcacca tgagcctctg gcagcccctg gtcctggtgc tcctggtgct |
| 61 | gggctgctgc tttgctgccc ccagacagcg ccagtccacc cttgtgctct tccctggaga |
| 121 | cctgagaacc aatctcaccg acaggcagct ggcagaggaa tacctgtacc gctatggtta |
| 181 | cactcgggtg gcagagatgc gtggagagtc gaaatctctg gggcctgcgc tgctgcttct |
| 241 | ccagaagcaa ctgtccctgc ccgagaccgg tgagctggat agcgccacgc tgaaggccat |
| 301 | gcgaacccca cggtgcgggg tcccagacct gggcagattc caaacctttg agggcgacct |
| 361 | caagtggcac caccacaaca tcacctattg gatccaaaac tactcggaag acttgccgcg |
| 421 | ggcggtgatt gacgacgcct ttgcccgcgc cttcgcactg tggagcgcgg tgacgccgct |
| 481 | caccttcact cgcgtgtaca gccgggacgc agacatcgtc atccagtttg gtgtcgcgga |
| 541 | gcacggagac gggtatccct tcgacgggaa ggacgggctc ctggcacacg cctttcctcc |
| 601 | tggccccggc attcagggag acgcccattt cgacgatgac gagttgtggt ccctgggcaa |
| 661 | gggcgtcgtg gttccaactc ggtttggaaa cgcagatggc gcggcctgcc acttcccctt |
| 721 | catcttcgag ggccgctcct actctgcctg caccaccgac ggtcgctccg acggcttgcc |
| 781 | ctggtgcagt accacggcca actacgacac cgacgaccgg tttggcttct gccccagcga |
| 841 | gagactctac acccaggacg gcaatgctga tgggaaaccc tgccagtttc cattcatctt |
| 901 | ccaaggccaa tcctactccg cctgcaccac ggacggtcgc tccgacggct accgctggtg |
| 961 | cgccaccacc gccaactacg accgggacaa gctcttcggc ttctgcccga cccgagctga |
| 1021 | ctcgacggtg atggggggca actcggcggg ggagctgtgc gtcttcccct tcactttcct |
| 1081 | gggtaaggag tactcgacct gtaccagcga gggccgcgga gatgggcgcc tctggtgcgc |
| 1141 | taccacctcg aactttgaca gcgacaagaa gtggggcttc tgcccggacc aaggatacag |
| 1201 | tttgttcctc gtggcggcgc atgagttcgg ccacgcgctg ggcttagatc attcctcagt |
| 1261 | gccggaggcg ctcatgtacc ctatgtaccg cttcactgag gggcccccct tgcataagga |
| 1321 | cgacgtgaat ggcatccggc acctctatgg tcctcgccct gaacctgagc cacggcctcc |
| 1381 | aaccaccacc acaccgcagc ccacggctcc cccgacggtc tgccccaccg gaccccccac |
| 1441 | tgtccacccc tcagagcgcc ccacagctgg ccccacaggt cccccctcag ctggccccac |
| 1501 | aggtcccccc actgctggcc cttctacggc cactactgtg cctttgagtc cggtggacga |
| 1561 | tgcctgcaac gtgaacatct tcgacgccat cgcggagatt gggaaccagc tgtatttgtt |
| 1621 | caaggatggg aagtactggc gattctctga gggcaggggg agccggccgc agggcccctt |
| 1681 | ccttatcgcc gacaagtggc ccgcgctgcc ccgcaagctg gactcggtct ttgaggagcg |
| 1741 | gctctccaag aagcttttct tcttctctgg gcgccaggtg tgggtgtaca caggcgcgtc |
| 1801 | ggtgctgggc ccgaggcgtc tggacaagct gggcctggga gccgacgtgg cccaggtgac |
| 1861 | cggggccctc cggagtggca gggggaagat gctgctgttc agcgggcggc gcctctggag |
| 1921 | gttcgacgtg aaggcgcaga tggtggatcc ccggagcgcc agcgaggtgg accggatgtt |
| 1981 | ccccggggtg cctttggaca cgcacgacgt cttccagtac cgagagaaag cctatttctg |
| 2041 | ccaggaccgc ttctactggc gcgtgagttc ccggagtgag ttgaaccagg tggaccaagt |
| 2101 | gggctacgtg acctatgaca tcctgcagtg ccctgaggac tagggctccc gtcctgcttt |
| 2161 | ggcagtgcca tgtaaatccc cactgggacc aaccctgggg aaggagccag tttgccggat |
| 2221 | acaaactggt attctgttct ggaggaaagg gaggagtgga ggtgggctgg gccctctctt |
| 2281 | ctcacctttg ttttttgttg gagtgtttct aataaacttg gattctctaa cctttaaaaa |
| 2341 | aaaaaaaaaa aaaaaaaaaa aaaaaaaaaa aaaaaaaaaa aaaaaaa |
| SEQ ID NO: 8 | |
| NP 004985 Human MMP-9 Preprotein | |
| 1 | mslwqplvlv llvlgccfaa prqrqstlvl fpgdlrtnlt drqlaeeyly rygytrvaem |
| 61 | rgeskslgpa llllqkqlsl petgeldsat lkamrtprcg vpdlgrfqtf egdlkwhhhn |
| 121 | itywiqnyse dlpravidda farafalwsa vtpltftrvy srdadiviqf gvaehgdgyp |
| 181 | fdgkdgllah afppgpgiqg dahfdddelw slgkgvvvpt rfgnadgaac hfpfifegrs |
| 241 | ysacttdgrs dglpwcstta nydtddrfgf cpserlytqd gnadgkpcqf pfifqgqsys |
| 301 | acttdgrsdg yrwcattany drdklfgfcp tradstvmgg nsagelcvfp ftflgkeyst |
| 361 | ctsegrgdgr lwcattsnfd sdkkwgfcpd qgyslflvaa hefghalgld hssvpealmy |
| 421 | pmyrftegpp lhkddvngir hlygprpepe prppttttpq ptapptvcpt gpptvhpser |
| 481 | ptagptgpps agptgpptag pstattvpls pvddacnvni fdaiaeignq lylfkdgkyw |
| 541 | rfsegrgsrp qgpfliadkw palprkldsv feerlskklf ffsgrqvwvy tgasvlgprr |
| 601 | ldklglgadv aqvtgalrsg rgkmllfsgr rlwrfdvkaq mvdprsasev drmfpgvpld |
| 661 | thdvfqyrek ayfcqdrfyw rvssrselnq vdqvgyvtyd ilqcped |
| SEQ ID NO: 9 | |
| NM 002425 Human MMP10 | |
| 1 | aaagaaggta agggcagtga gaatgatgca tcttgcattc cttgtgctgt tgtgtctgcc |
| 61 | agtctgctct gcctatcctc tgagtggggc agcaaaagag gaggactcca acaaggatct |
| 121 | tgcccagcaa tacctagaaa agtactacaa cctcgaaaag gatgtgaaac agtttagaag |
| 181 | aaaggacagt aatctcattg ttaaaaaaat ccaaggaatg cagaagttcc ttgggttgga |
| 241 | ggtgacaggg aagctagaca ctgacactct ggaggtgatg cgcaagccca ggtgtggagt |
| 301 | tcctgacgtt ggtcacttca gctcctttcc tggcatgccg aagtggagga aaacccacct |
| 361 | tacatacagg attgtgaatt atacaccaga tttgccaaga gatgctgttg attctgccat |
| 421 | tgagaaagct ctgaaagtct gggaagaggt gactccactc acattctcca ggctgtatga |
| 481 | aggagaggct gatataatga tctctttcgc agttaaagaa catggagact tttactcttt |
| 541 | tgatggccca ggacacagtt tggctcatgc ctacccacct ggacctgggc tttatggaga |
| 601 | tattcacttt gatgatgatg aaaaatggac agaagatgca tcaggcacca atttattcct |
| 661 | cgttgctgct catgaacttg gccactccct ggggctcttt cactcagcca acactgaagc |
| 721 | tttgatgtac ccactctaca actcattcac agagctcgcc cagttccgcc tttcgcaaga |
| 781 | tgatgtgaat ggcattcagt ctctctacgg acctccccct gcctctactg aggaacccct |
| 841 | ggtgcccaca aaatctgttc cttcgggatc tgagatgcca gccaagtgtg atcctgcttt |
| 901 | gtccttcgat gccatcagca ctctgagggg agaatatctg ttctttaaag acagatattt |
| 961 | ttggcgaaga tcccactgga accctgaacc tgaatttcat ttgatttctg cattttggcc |
| 1021 | ctctcttcca tcatatttgg atgctgcata tgaagttaac agcagggaca ccgtttttat |
| 1081 | ttttaaagga aatgagttct gggccatcag aggaaatgag gtacaagcag gttatccaag |
| 1141 | aggcatccat accctgggtt ttcctccaac cataaggaaa attgatgcag ctgtttctga |
| 1201 | caaggaaaag aagaaaacat acttctttgc agcggacaaa tactggagat ttgatgaaaa |
| 1261 | tagccagtcc atggagcaag gcttccctag actaatagct gatgactttc caggagttga |
| 1321 | gcctaaggtt gatgctgtat tacaggcatt tggatttttc tacttcttca gtggatcatc |
| 1381 | acagtttgag tttgacccca atgccaggat ggtgacacac atattaaaga gtaacagctg |
| 1441 | gttacattgc taggcgagat agggggaaga cagatatggg tgtttttaat aaatctaata |
| 1501 | attattcatc taatgtatta tgagccaaaa tggttaattt ttcctgcatg ttctgtgact |
| 1561 | gaagaagatg agccttgcag atatctgcat gtgtcatgaa gaatgtttct ggaattcttc |
| 1621 | acttgctttt gaattgcact gaacagaatt aagaaatact catgtgcaat aggtgagaga |
| 1681 | atgtattttc atagatgtgt tattacttcc tcaataaaaa gttttatttt gggcctgttc |
| 1741 | ctt |
| SEQ ID NO: 10 | |
| NP 002416 Human MMP10 preprotein | |
| 1 | mmhlaflvll clpvcsaypl sgaakeedsn kdlaqqylek yynlekdvkq frrkdsnliv |
| 61 | kkiqgmqkfl glevtgkldt cdtlevmrkpr cgvpdvghfs sfpgmpkwrk thltyrivny |
| 121 | tpdlprdavd saiekalkvw eevtpltfsr lyegeadimi sfavkehgdf ysfdgpghsl |
| 181 | ahayppgpgl ygdihfddde kwtedasgtn lflvaahelg hslglfhsan tealmyplyn |
| 241 | sftelaqfrl sqddvngiqs lygpppaste eplvptksvp sgsempakcd palsfdaist |
| 301 | lrgeylffkd ryfwrrshwn pepefhlisa fwpslpsyld aayevnsrdt vfifkgnefw |
| 361 | airgnevqag yprgihtlgf pptirkidaa vsdkekkkty ffaadkywrf densqsmeqg |
| 421 | fprliaddfp gvepkvdavl qafgffyffs gssqfefdpn armvthilks nswlhc |
| SEQ ID NO: 11 | |
| NM 004995 Human MMP14- | |
| 1 | cagaccccag ttcgccgact aagcagaaga aagatcaaaa accggaaaag aggagaagag |
| 61 | caaacaggca ctttgaggaa caatcccctt taactccaag ccgacagcgg tctaggaatt |
| 121 | caagttcagt gcctaccgaa gacaaaggcg ccccgaggga gtggcggtgc gaccccaggg |
| 181 | cgtgggcccg gccgcggagc ccacactgcc cggctgaccc ggtggtctcg gaccatgtct |
| 241 | cccgccccaa gacccccccg ttgtctcctg ctccccctgc tcacgctcgg caccgcgctc |
| 301 | gcctccctcg gctcggccca aagcagcagc ttcagccccg aagcctggct acagcaatat |
| 361 | ggctacctgc ctcccgggga cctacgtacc cacacacagc gctcacccca gtcactctca |
| 421 | gcggccatcg ctgccatgca gaagttttac ggcttgcaag taacaggcaa agctgatgca |
| 481 | gacaccatga aggccatgag gcgcccccga tgtggtgttc cagacaagtt tggggctgag |
| 541 | atcaaggcca atgttcgaag gaagcgctac gccatccagg gtctcaaatg gcaacataat |
| 601 | gaaatcactt tctgcatcca gaattacacc cccaaggtgg gcgagtatgc cacatacgag |
| 661 | gccattcgca aggcgttccg cgtgtgggag agtgccacac cactgcgctt ccgcgaggtg |
| 721 | ccctatgcct acatccgtga gggccatgag aagcaggccg acatcatgat cttctttgcc |
| 781 | gagggcttcc atggcgacag cacgcccttc gatggtgagg gcggcttcct ggcccatgcc |
| 841 | tacttcccag gccccaacat tggaggagac acccactttg actctgccga gccttggact |
| 901 | gtcaggaatg aggatctgaa tggaaatgac atcttcctgg tggctgtgca cgagctgggc |
| 961 | catgccctgg ggctcgagca ttccagtgac ccctcggcca tcatggcacc cttttaccag |
| 1021 | tggatggaca cggagaattt tgtgctgccc gatgatgacc gccggggcat ccagcaactt |
| 1081 | tatgggggtg agtcagggtt ccccaccaag atgccccctc aacccaggac tacctcccgg |
| 1141 | ccttctgttc ctgataaacc caaaaacccc acctatgggc ccaacatctg tgacgggaac |
| 1201 | tttgacaccg tggccatgct ccgaggggag atgtttgtct tcaaggagcg ctggttctgg |
| 1261 | cgggtgagga ataaccaagt gatggatgga tacccaatgc ccattggcca gttctggcgg |
| 1321 | ggcctgcctg cgtccatcaa cactgcctac gagaggaagg atggcaaatt cgtcttcttc |
| 1381 | aaaggagaca agcattgggt gtttgatgag gcgtccctgg aacctggcta ccccaagcac |
| 1441 | attaaggagc tgggccgagg gctgcctacc gacaagattg atgctgctct cttctggatg |
| 1501 | cccaatggaa agacctactt cttccgtgga aacaagtact accgtttcaa cgaagagctc |
| 1561 | agggcagtgg atagcgagta ccccaagaac atcaaagtct gggaagggat ccctgagtct |
| 1621 | cccagagggt cattcatggg cagcgatgaa gtcttcactt acttctacaa ggggaacaaa |
| 1681 | tactggaaat tcaacaacca gaagctgaag gtagaaccgg gctaccccaa gtcagccctg |
| 1741 | agggactgga tgggctgccc atcgggaggc cggccggatg aggggactga ggaggagacg |
| 1801 | gaggtgatca tcattgaggt ggacgaggag ggcggcgggg cggtgagcgc ggctgccgtg |
| 1861 | gtgctgcccg tgctgctgct gctcctggtg ctggcggtgg gccttgcagt cttcttcttc |
| 1921 | agacgccatg ggacccccag gcgactgctc tactgccagc gttccctgct ggacaaggtc |
| 1981 | tgacgcccac cgccggcccg cccactccta ccacaaggac tttgcctctg aaggccagtg |
| 2041 | gcagcaggtg gtggtgggtg ggctgctccc atcgtcccga gccccctccc cgcagcctcc |
| 2101 | ttgcttctct ctgtcccctg gctggcctcc ttcaccctga ccgcctccct ccctcctgcc |
| 2161 | ccggcattgc atcttcccta gataggtccc ctgagggctg agtgggaggg cggccctttc |
| 2221 | cagcctctgc ccctcagggg aaccctgtag ctttgtgtct gtccagcccc atctgaatgt |
| 2281 | gttgggggct ctgcacttga aggcaggacc ctcagacctc gctggtaaag gtcaaatggg |
| 2341 | gtcatctgct ccttttccat cccctgacat accttaacct ctgaactctg acctcaggag |
| 2401 | gctctgggca ctccagccct gaaagcccca ggtgtaccca attggcagcc tctcactact |
| 2461 | ctttctggct aaaaggaatc taatcttgtt gagggtagag accctgagac agtgtgaggg |
| 2521 | ggtggggact gccaagccac cctaagacct tgggaggaaa actcagagag ggtcttcgtt |
| 2581 | gctcagtcag tcaagttcct cggagatctg cctctgcctc acctacccca gggaacttcc |
| 2641 | aaggaaggag cctgagccac tggggactaa gtgggcagaa gaaacccttg gcagccctgt |
| 2701 | gcctctcgaa tgttagcctt ggatggggct ttcacagtta gaagagctga aaccaggggt |
| 2761 | gcagctgtca ggtagggtgg ggccggtggg agaggcccgg gtcagagccc tgggggtgag |
| 2821 | cctgaaggcc acagagaaag aaccttgccc aaactcaggc agctggggct gaggcccaaa |
| 2881 | ggcagaacag ccagaggggg caggagggga ccaaaaagga aaatgaggac gtgcagcagc |
| 2941 | attggaaggc tggggccggg caggccaggc caagccaagc agggggccac agggtgggct |
| 3001 | gtggagctct caggaagggc cctgaggaag gcacacttgc tcctgttggt ccctgtcctt |
| 3061 | gctgcccagg cagcgtggag gggaagggta gggcagccag agaaaggagc agagaaggca |
| 3121 | cacaaacgag gaatgagggg cttcacgaga ggccacaggg cctggctggc cacgctgtcc |
| 3181 | cggcctgctc accatctcag tgaggggcag gagctggggc tcgcttaggc tgggtccacg |
| 3241 | cttccctggt gccagcaccc ctcaagcctg tctcaccagt ggcctgccct ctcgctcccc |
| 3301 | cacccagccc acccattgaa gtctccttgg gccaccaaag gtggtggcca tggtaccggg |
| 3361 | gacttgggag agtgagaccc agtggaggga gcaagaggag agggatgtcg ggggggtggg |
| 3421 | gcacggggta ggggaaatgg ggtgaacggt gctggcagtt cggctagatt tctgtcttgt |
| 3481 | ttgttttttt gttttgttta atgtatattt ttattataat tattatatat gaattccaaa |
| 3541 | aaaaaaaaaa aaaaaaaa |
| SEQ ID NO: 12 | |
| NP 004986 Human MMP-14 preprotein | |
| 1 | mspaprpprc lllplltlgt alaslgsaqs ssfspeawlq qygylppgdl rthtqrspqs |
| 61 | lsaaiaamqk fyglqvtgka dadtmkamrr prcgvpdkfg aeikanvrrk ryaiqglkwq |
| 121 | hneitfciqn ytpkvgeyat yeairkafrv wesatplrfr evpyayireg hekqadimif |
| 181 | faegfhgdst pfdgegglfa hayfpgpnig gdthfdsaep wtvrnedlng ndiflvavhe |
| 241 | lghalglehs sdpsaimapf yqwmdtenfv lpdddrrgiq qlyggesgfp tkmppqprtt |
| 301 | srpsvpdkpk nptygpnicd gnfdtvamlr gemfvfkerw fwrvrnnqvm dgypmpigqf |
| 361 | wrglpasint ayerkdgkfv ffkgdkhwvf deaslepgyp khikelgrgl ptdkidaalf |
| 421 | wmpngktyff rgnkyyrfne elravdseyp knikvwegip esprgsfmgs devftyfykg |
| 481 | nkywkfnnqk lkvepgypks alrdwmgcps ggrpdegtee eteviiievd eegggavsaa |
| 541 | avvlpvllll lvlavglavf ffrrhgtprr llycqrslld kv |
| SEQ ID NO: 13 | |
| NM 003255 Human TIMP-2 | |
| 1 | cgcagcaaac acatccgtag aaggcagcgc ggccgccgag aaccgcagcg ccgctcgccc |
| 61 | gccgcccccc accccgccgc cccgcccggc gaattgcgcc ccgcgcccct cccctcgcgc |
| 121 | ccccgagaca aagaggagag aaagtttgcg cggccgagcg gggcaggtga ggagggtgag |
| 181 | ccgcgcggga ggggcccgcc tcggccccgg ctcagccccc gcccgcgccc ccagcccgcc |
| 241 | gccgcgagca gcgcccggac cccccagcgg cggcccccgc ccgcccagcc ccccggcccg |
| 301 | ccatgggcgc cgcggcccgc accctgcggc tggcgctcgg cctcctgctg ctggcgacgc |
| 361 | tgcttcgccc ggccgacgcc tgcagctgct ccccggtgca cccgcaacag gcgttttgca |
| 421 | atgcagatgt agtgatcagg gccaaagcgg tcagtgagaa ggaagtggac tctggaaacg |
| 481 | acatttatgg caaccctatc aagaggatcc agtatgagat caagcagata aagatgttca |
| 541 | aagggcctga gaaggatata gagtttatct acacggcccc ctcctcggca gtgtgtgggg |
| 601 | tctcgctgga cgttggagga aagaaggaat atctcattgc aggaaaggcc gagggggacg |
| 661 | gcaagatgca catcaccctc tgtgacttca tcgtgccctg ggacaccctg agcaccaccc |
| 721 | agaagaagag cctgaaccac aggtaccaga tgggctgcga gtgcaagatc acgcgctgcc |
| 781 | ccatgatccc gtgctacatc tcctccccgg acgagtgcct ctggatggac tgggtcacag |
| 841 | agaagaacat caacgggcac caggccaagt tcttcgcctg catcaagaga agtgacggct |
| 901 | cctgtgcgtg gtaccgcggc gcggcgcccc ccaagcagga gtttctcgac atcgaggacc |
| 961 | cataagcagg cctccaacgc ccctgtggcc aactgcaaaa aaagcctcca agggtttcga |
| 1021 | ctggtccagc tctgacatcc cttcctggaa acagcatgaa taaaacactc atcccatggg |
| 1081 | tccaaattaa tatgattctg ctcccccctt ctccttttag acatggttgt gggtctggag |
| 1141 | ggagacgtgg gtccaaggtc ctcatcccat cctccctctg ccaggcacta tgtgtctggg |
| 1201 | gcttcgatcc ttgggtgcag gcagggctgg gacacgcggc ttccctccca gtccctgcct |
| 1261 | tggcaccgtc acagatgcca agcaggcagc acttagggat ctcccagctg ggttagggca |
| 1321 | gggcctggaa atgtgcattt tgcagaaact tttgagggtc gttgcaagac tgtgtagcag |
| 1381 | gcctaccagg tccctttcat cttgagaggg acatggccct tgtttcctgc agcttccacg |
| 1441 | cctctgcact ccctgcccct ggcaagtgct cccatcgccc cggtgcccac catgagctcc |
| 1501 | cagcacctga ctccccccac atccaagggc agcctggaac cagtggctag ttcttgaagg |
| 1561 | agccccatca atcctattaa tcctcagaat tccagtggga gcctccctct gagccttgta |
| 1621 | gaaatgggag cgagaaaccc cagctgagct gcgttccagc ctcagctgag tctttttggt |
| 1681 | ctgcacccac ccccccaccc cccccccccc gcccacatgc tccccagctt gcaggaggaa |
| 1741 | tcggtgaggt cctgtcctga ggctgctgtc cggggccggt ggctgccctc aaggtccctt |
| 1801 | ccctagctgc tgcggttgcc attgcttctt gcctgttctg gcatcaggca cctggattga |
| 1861 | gttgcacagc tttgctttat ccgggcttgt gtgcagggcc cggctgggct ccccatctgc |
| 1921 | acatcctgag gacagaaaaa gctgggtctt gctgtgccct cccaggctta gtgttccctc |
| 1981 | cctcaaagac tgacagccat cgttctgcac ggggctttct gcatgtgacg ccagctaagc |
| 2041 | atagtaagaa gtccagccta ggaagggaag gattttggag gtaggtggct ttggtgacac |
| 2101 | actcacttct ttctcagcct ccaggacact atggcctgtt ttaagagaca tcttattttt |
| 2161 | ctaaaggtga attctcagat gataggtgaa cctgagttgc agatatacca acttctgctt |
| 2221 | gtatttctta aatgacaaag attacctagc taagaaactt cctagggaac tagggaacct |
| 2281 | atgtgttccc tcagtgtggt ttcctgaagc cagtgatatg ggggttagga taggaagaac |
| 2341 | tttctcggta atgataagga gaatctcttg tttcctccca cctgtgttgt aaagataaac |
| 2401 | tgacgatata caggcacatt atgtaaacat acacacgcaa tgaaaccgaa gcttggcggc |
| 2461 | ctgggcgtgg tcttgcaaaa tgcttccaaa gccaccttag cctgttctat tcagcggcaa |
| 2521 | ccccaaagca cctgttaaga ctcctgaccc ccaagtggca tgcagccccc atgcccaccg |
| 2581 | ggacctggtc agcacagatc ttgatgactt ccctttctag ggcagactgg gagggtatcc |
| 2641 | aggaatcggc ccctgcccca cgggcgtttt catgctgtac agtgacctaa agttggtaag |
| 2701 | atgtcataat ggaccagtcc atgtgatttc agtatataca actccaccag acccctccaa |
| 2761 | cccatataac accccacccc tgttcgcttc ctgtatggtg atatcatatg taacatttac |
| 2821 | tcctgtttct gctgattgtt tttttaatgt tttggtttgt ttttgacatc agctgtaatc |
| 2881 | attcctgtgc tgtgtttttt attacccttg gtaggtatta gacttgcact tttttaaaaa |
| 2941 | aaggtttctg catcgtggaa gcatttgacc cagagtggaa cgcgtggcct atgcaggtgg |
| 3001 | attccttcag gtctttcctt tggttctttg agcatctttg ctttcattcg tctcccgtct |
| 3061 | ttggttctcc agttcaaatt attgcaaagt aaaggatctt tgagtaggtt cggtctgaaa |
| 3121 | ggtgtggcct ttatatttga tccacacacg ttggtctttt aaccgtgctg agcagaaaac |
| 3181 | aaaacaggtt aagaagagcc gggtggcagc tgacagagga agccgctcaa ataccttcac |
| 3241 | aataaatagt ggcaatatat atatagttta agaaggctct ccatttggca tcgtttaatt |
| 3301 | tatatgttat gttctaagca cagctctctt ctcctatttt catcctgcaa gcaactcaaa |
| 3361 | atatttaaaa taaagtttac attgtagtta ttttcaaatc tttgcttgat aagtattaag |
| 3421 | aaatattgga cttgctgccg taatttaaag ctctgttgat tttgtttccg tttggatttt |
| 3481 | tgggggaggg gagcactgtg tttatgctgg aatatgaagt ctgagacctt ccggtgctgg |
| 3541 | gaacacacaa gagttgttga aagttgacaa gcagactgcg catgtctctg atgctttgta |
| 3601 | tcattcttga gcaatcgctc ggtccgtgga caataaacag tattatcaaa gagaaaaaaa |
| 3661 | aaaaaaaaaa |
| SEQ ID NO: 14 | |
| NP 003246 Human TIMP-2 precursor | |
| 1 | mgaaartlrl algllllatl lrpadacscs pvhpqqafcn advvirakav sekevdsgnd |
| 61 | iygnpikriq yeikqikmfk gpekdiefiy tapssavcgv sldvggkkey liagkaegdg |
| 121 | kmhitlcdfi vpwdtlsttq kkslnhryqm gceckitrcp mipcyisspd eclwmdwvte |
| 181 | kninghqakf facikrsdgs cawyrgaapp kqefldiedp |
| SEQ ID NO: 15 | |
| NM 013599 Murine MMP-9 | |
| 1 | ctcaccatga gtccctggca gcccctgctc ctggctctcc tggctttcgg ctgcagctct |
| 61 | gctgcccctt accagcgcca gccgactttt gtggtcttcc ccaaagacct gaaaacctcc |
| 121 | aacctcacgg acacccagct ggcagaggca tacttgtacc gctatggtta cacccgggcc |
| 181 | gcccagatga tgggagagaa gcagtctcta cggccggctt tgctgatgct tcagaagcag |
| 241 | ctctccctgc cccagactgg tgagctggac agccagacac taaaggccat tcgaacacca |
| 301 | cgctgtggtg tcccagacgt gggtcgattc caaaccttca aaggcctcaa gtgggaccat |
| 361 | cataacatca catactggat ccaaaactac tctgaagact tgccgcgaga catgatcgat |
| 421 | gacgccttcg cgcgcgcctt cgcggtgtgg ggcgaggtgg cacccctcac cttcacccgc |
| 481 | gtgtacggac ccgaagcgga cattgtcatc cagtttggtg tcgcggagca cggagacggg |
| 541 | tatcccttcg acggcaagga cggccttctg gcacacgcct ttccccctgg cgccggcgtt |
| 601 | cagggagatg cccatttcga cgacgacgag ttgtggtcgc tgggcaaagg cgtcgtgatc |
| 661 | cccacttact atggaaactc aaatggtgcc ccatgtcact ttcccttcac cttcgaggga |
| 721 | cgctcctatt cggcctgcac cacagacggc cgcaacgacg gcacgccttg gtgtagcaca |
| 781 | acagctgact acgataagga cggcaaattt ggtttctgcc ctagtgagag actctacacg |
| 841 | gagcacggca acggagaagg caaaccctgt gtgttcccgt tcatctttga gggccgctcc |
| 901 | tactctgcct gcaccactaa aggccgctcg gatggttacc gctggtgcgc caccacagcc |
| 961 | aactatgacc aggataaact gtatggcttc tgccctaccc gagtggacgc gaccgtagtt |
| 1021 | gggggcaact cggcaggaga gctgtgcgtc ttccccttcg tcttcctggg caagcagtac |
| 1081 | tcttcctgta ccagcgacgg ccgcagggat gggcgcctct ggtgtgcgac cacatcgaac |
| 1141 | ttcgacactg acaagaagtg gggtttctgt ccagaccaag ggtacagcct gttcctggtg |
| 1201 | gcagcgcacg agttcggcca tgcactgggc ttagatcatt ccagcgtgcc ggaagcgctc |
| 1261 | atgtacccgc tgtatagcta cctcgagggc ttccctctga ataaagacga catagacggc |
| 1321 | atccagtatc tgtatggtcg tggctctaag cctgacccaa ggcctccagc caccaccaca |
| 1381 | actgaaccac agccgacagc acctcccact atgtgtccca ctatacctcc cacggcctat |
| 1441 | cccacagtgg gccccacggt tggccctaca ggcgccccct cacctggccc cacaagcagc |
| 1501 | ccgtcacctg gccctacagg cgccccctca cctggcccta cagcgccccc tactgcgggc |
| 1561 | tcttctgagg cctctacaga gtctttgagt ccggcagaca atccttgcaa tgtggatgtt |
| 1621 | tttgatgcta ttgctgagat ccagggcgct ctgcatttct tcaaggacgg ttggtactgg |
| 1681 | aagttcctga atcatagagg aagcccatta cagggcccct tccttactgc ccgcacgtgg |
| 1741 | ccagccctgc ctgcaacgct ggactccgcc tttgaggatc cgcagaccaa gagggttttc |
| 1801 | ttcttctctg gacgtcaaat gtgggtgtac acaggcaaga ccgtgctggg ccccaggagt |
| 1861 | ctggataagt tgggtctagg cccagaggta acccacgtca gcgggcttct cccgcgtcgt |
| 1921 | ctcgggaagg ctctgctgtt cagcaagggg cgtgtctgga gattcgactt gaagtctcag |
| 1981 | aaggtggatc cccagagcgt cattcgcgtg gataaggagt tctctggtgt gccctggaac |
| 2041 | tcacacgaca tcttccagta ccaagacaaa gcctatttct gccatggcaa attcttctgg |
| 2101 | cgtgtgagtt tccaaaatga ggtgaacaag gtggaccatg aggtgaacca ggtggacgac |
| 2161 | gtgggctacg tgacctacga cctcctgcag tgcccttgaa ctagggctcc ttctttgctt |
| 2221 | caaccgtgca gtgcaagtct ctagagacca ccaccaccac caccacacac aaaccccatc |
| 2281 | cgagggaaag gtgctagctg gccaggtaca gactggtgat ctcttctaga gactgggaag |
| 2341 | gagtggaggc aggcagggct ctctctgccc accgtccttt cttgttggac tgtttctaat |
| 2401 | aaacacggat ccccaacctt ttccagctac tttagtcaat cagcttatct gtagttgcag |
| 2461 | atgcatccga gcaagaagac aactttgtag ggtggattct gaccttttat ttttgtgtgg |
| 2521 | cgtctgagaa ttgaatcagc tggcttttgt gacaggcact tcaccggcta aaccacctct |
| 2581 | cccgactcca gcccttttat ttattatgta tgaggttatg ttcacatgca tgtatttaac |
| 2641 | ccacagaatg cttactgtgt gtcgggcgcg gctccaaccg cgtcataaat attaaggtat |
| 2701 | tcagttgccc ctactggaag gtattatgta actatttctc tcttacattg gagaacacca |
| 2761 | ccgagctatc cactcatcaa acatttattg agagcatccc tagggagcca ggctctctac |
| 2821 | tgggcgttag ggacagaaat gttggttctt ccttcaagga ttgctcagag attctccgtg |
| 2881 | tcctgtaaat ctgctgaaac cagaccccag actcctctct ctcccgagag tccaactcac |
| 2941 | tcactgtggt tgctggcagc tgcagcatgc gtatacagca tgtgtgctag agaggtagag |
| 3001 | ggggtctgtg cgttatggtt caggtcagac tgtgtcctcc aggtgagatg acccctcagc |
| 3061 | tggaactgat ccaggaagga taaccaagtg tcttcctggc agtctttttt aaataaatga |
| 3121 | ataaatgaat atttacttaa aaaaaaaaaa aaaaaaaaaa aaaaaaaaaa aaaaaaaaaa |
| 3181 | aaaaa |
| SEQ ID NO: 16 | |
| NP 038627 Murine MMP-9 | |
| 1 | mspwqpllla llafgcssaa pyqrqptfvv fpkdlktsnl tdtqlaeayl yrygytraaq |
| 61 | mmgekqslrp allmlqkqls lpqtgeldsq tlkairtprc gvpdvgrfqt fkglkwdhhn |
| 121 | itywiqnyse dlprdmidda farafavwge vapltftrvy gpeadiviqf gvaehgdgyp |
| 181 | fdgkdgllah afppgagvqg dahfdddelw slgkgvvipt yygnsngapc hfpftfegrs |
| 241 | ysacttdgrn dgtpwcstta dydkdgkfgf cpserlyteh gngegkpcvf pfifegrsys |
| 301 | acttkgrsdg yrwcattany dqdklygfcp trvdatvvgg nsagelcvfp fvflgkqyss |
| 361 | ctsdgrrdgr lwcattsnfd tdkkwgfcpd qgyslflvaa hefghalgid hssvpealmy |
| 421 | plysylegfp lnkddidgiq ylygrgskpd prppatttte pqptapptmc ptipptaypt |
| 481 | vgptvgptga pspgptssps pgptgapspg ptapptagss easteslspa dnpcnvdvfd |
| 541 | aiaeiqgalh ffkdgwywkf lnhrgsplqg pfltartwpa lpatldsafe dpqtkrvfff |
| 601 | sgrqmwvytg ktvlgprsld klglgpevth vsgllprrlg kallfskgrv wrfdlksqkv |
| 661 | dpqsvirvdk efsgvpwnsh difqyqdkay fchgkffwrv sfqnevnkvd hevnqvddvg |
| 721 | yvtydllqcp |
| SEQ ID NO: 17 | |
| NM 019471 Murine MMP-10 | |
| 1 | ggcacgaaga aggatcggtt tttaaagtaa agactgtctg tatggagcca ctagccatcc |
| 61 | tggcactgct gagcctacca atctgctcag cgtatcctct gcatggggca gtgacacaag |
| 121 | gccacccaag catggatctt gctcagcaat acctagaaaa atactacaac tttaaaaaaa |
| 181 | atgagaaaca aattttcaaa agaaaggaca gtagtcctgt tgtcaaaaaa attcaagaaa |
| 241 | tgcagaagtt cctcgggttg gagatgacag ggaagctgga ctccaacact atggagctga |
| 301 | tgcacaagcc caggtgtggt gttcctgatg ttggtggctt cagtaccttc ccaggttcgc |
| 361 | caaaatggag gaaatcccac atcacctaca ggattgtgaa ttatacacca gatttgccaa |
| 421 | gacagagtgt ggattctgcc attgagaaag ctttgaaggt ctgggaggag gtgaccccac |
| 481 | tcactttctc caggatctct gaaggagagg ctgacataat gatctccttt gcagttggag |
| 541 | aacacggaga cttttaccct tttgatgggc caggacagag tctggctcat gcctacccac |
| 601 | ctggccctgg attttatgga gatgttcact tcgatgatga tgagaaatgg acacttgcac |
| 661 | cctcagggac caacttattc ctggttgcag cccatgaact tggccactcc ctgggtctct |
| 721 | ttcattccga caagaaagaa tctctgatgt acccagtcta caggttctcc acaagcccag |
| 781 | ctaacttcca cctttctcaa gatgatatag agggcattca atccctgtat ggagccggtc |
| 841 | cctcctccga tgccacagtg gttcctgtgt tgtctgtctc tccaagacct gagaccccag |
| 901 | acaaatgtga tcctgctttg tcctttgatt cagtcagcac gctgagaggg gaagtcctat |
| 961 | tctttaaaga caggtacttc tggcgcagat cccattggaa tcccgagcct gaatttcatt |
| 1021 | tgatatcagc attttggccc actcttcctt cagacttaga tgctgcctat gaggctcaca |
| 1081 | acacggacag tgttctgatt tttaaaggaa gtcagttctg ggcagtccga ggaaatgaag |
| 1141 | tccaagcagg ctacccaaag gggatccaca ctctcggttt tcctcccacc gtgaagaaga |
| 1201 | ttgatgcagc tgtttttgaa aaggagaaga agaaaacgta cttctttgta ggggacaaat |
| 1261 | actggagatt tgatgagaca agacatgtta tggataaagg cttcccaaga cagataacag |
| 1321 | atgattttcc aggaattgag ccacaagttg atgctgtgtt acacgaattt gggttttttt |
| 1381 | atttcttccg aggatcatca cagttcgagt ttgaccccaa tgccaggacg gtgacacaca |
| 1441 | tactgaagag caacagctgg ctgctgtgct gatcatcatg acaagacata tacaaccctg |
| 1501 | taaaatgagt ctcatgactt cccacctact ttattatgtg tcagaatgat tagttgctcc |
| 1561 | tgcatgttct gtggctccgg atgagcgcag cagatgtctt tcataatgag tcacaaagca |
| 1621 | tcacctgagc acagaagtga aattttctca ctgcagtagg tgagaggatg catccatccc |
| 1681 | catgggtatt ttattattta ataaagagct ttatttttga a |
| SEQ ID NO: 18 | |
| NP 062344 Murine MMP-10 | |
| 1 | meplailall slpicsaypl hgavtqghps mdlaqqylek yynfkknekq ifkrkdsspv |
| 61 | vkkiqemqkf lglemtgkld sntmelmhkp rcgvpdvggf stfpgspkwr kshityrivn |
| 121 | ytpdlprqsv dsaiekalkv weevtpltfs risegeadim isfavgehgd fypfdgpgqs |
| 181 | lahayppgpg fygdvhfddd ekwtlapsgt nlflvaahel ghslglfhsd kkeslmypvy |
| 241 | rfstspanfh lsqddiegiq slygagpssd atvvpvlsvs prpetpdkcd palsfdsvst |
| 301 | lrgevlffkd ryfwrrshwn pepefhlisa fwptlpsdld aayeahntds vlifkgsqfw |
| 361 | avrgnevqag ypkgihtlgf pptvkkidaa vfekekkkty ffvgdkywrf detrhvmdkg |
| 421 | fprqitddfp giepqvdavl hefgffyffr gssqfefdpn artvthilks nswllc |
| SEQ ID NO: 19 | |
| NM 008607 Murine MMP-13 | |
| 1 | gctgggcacc atgcattcag ctatcctggc caccttcttc ttgttgagct ggactccctg |
| 61 | ttggtccctg ccccttccct atggtgatga tgatgatgat gacctgtctg aggaagacct |
| 121 | tgtgtttgca gagcactact tgaaatcata ctaccatcct gcgactcttg cgggaatcct |
| 181 | gaagaagtct acagtgacct ccacagttga caggctccga gaaatgcaat ctttctttgg |
| 241 | cttagaggtg actggcaaac ttgatgatcc caccttagac atcatgagaa aaccaagatg |
| 301 | tggagtgcct gatgtgggtg aatacaatgt tttccctaga acactcaaat ggtcccaaac |
| 361 | gaacttaact tacaggattg tgaactatac tcctgatatg tcccattctg aagtggagaa |
| 421 | ggccttcaga aaagccttca aggtctggtc tgatgtgaca ccactgaatt tcaccagaat |
| 481 | ctatgatggc actgctgaca tcatgatatc ttttgggact aaagaacatg gtgacttcta |
| 541 | cccatttgat ggaccttctg gtcttctggc acacgctttt cctcctggac caaactatgg |
| 601 | tggggatgcc cattttgatg atgatgaaac ctggacaagc agttccaaag gctacaactt |
| 661 | gtttattgtt gctgcccatg agcttggcca ctccctaggt ctggatcact ccaaggaccc |
| 721 | aggagccctg atgtttccca tctataccta cactggcaaa agccatttca tgcttcctga |
| 781 | tgatgacgtt caaggaattc agtttcttta tggtccaggc gatgaagacc ccaaccctaa |
| 841 | gcatcccaaa acaccagaga agtgtgaccc agccctatcc cttgatgcca ttaccagtct |
| 901 | ccgaggagaa actatgatct ttaaagacag attcttctgg cgcctgcacc ctcagcaggt |
| 961 | tgaggctgag ctctttttga caaagtcctt ttggccagaa cttcccaacc atgtggatgc |
| 1021 | tgcatatgaa catccatccc gtgaccttat gtttatcttt agagggagaa aattctgggc |
| 1081 | tctgaatggt tatgacattc tggaaggtta tcccagaaaa atatctgacc tgggattccc |
| 1141 | aaaagaggtg aagagactga gcgctgcggt tcactttgag aacacgggga agaccctctt |
| 1201 | cttctctgag aaccacgtgt ggagttatga tgatgttaac cagactatgg acaaagatta |
| 1261 | tccccgcctc atagaagagg aattccctgg aattggcaac aaagtagatg ctgtctatga |
| 1321 | gaaaaatggc tatatctact ttttcaatgg gcccatacag tttgaataca gtatctggag |
| 1381 | taatcgcatt gtgagagtca tgccaacaaa ttccatattg tggtgttaag catctttaaa |
| 1441 | agttgttatt tatctcccag agagtatttg gaatactttc agatgtatgg ggtgggggtg |
| 1501 | gggtggagat atcaggggag agcttagttc tgtgaacgag cttcagtaag ttatctttga |
| 1561 | gcatacagta tctatatgac tatgcgtggc tggaaccaca tggaagaatt ttaaagtaat |
| 1621 | gcaattgaga accccaagga tcacctgatt cttgcgtgct atgaagaaac aagattgata |
| 1681 | ataacccaca gcaaacatgg ggtccatctg cttttgagag catgcataat tattaatata |
| 1741 | tttattttaa aaagcctaac agacataaaa taaatcatat ttatataact gaattgtctt |
| 1801 | tacaaaaaag tataaactta gaaacttgaa aattgtgagg agttcatgta tggggagcca |
| 1861 | cagatgagca cagataaagg gaaatgccta aaaaatgcac gttaacggac aactttccaa |
| 1921 | agagagattt cagcttttca ctgcgagcgt tcagatttac atccactttt atacaaccaa |
| 1981 | taaaaaaata ccaaagtcac taaagaaagg ggataacagc cactacaagg acagtggagg |
| 2041 | tggccttaca tttggcttaa tttttatgtt ggtcattact caaggctatg cacactggta |
| 2101 | gaagatattg agagagaaat ggaggagatt tctcttttta ttaaatattt aggcattgaa |
| 2161 | aagaccatag tgtgaaaagt caaaattgct ataagatacg taagcaatgc catagctttt |
| 2221 | tcatgaatta tttgactatt ttagaataaa actaatgttt caaccttgtt tatctaccca |
| 2281 | cttgttctaa tgacctatag actctttgat acatagtctc ttttctagta acttgtgtga |
| 2341 | caggggctaa ggcagaaata ttatgtagaa gtagatccag ctaagacaca gcaagccaga |
| 2401 | ataaagactg tgccagctgg tcagtcgccc ttttgagacc actcctttgt gctccaccat |
| 2461 | gtttgttaat ccctctctgc tttccttagc gagtaacact tggtgcttac tgatgtgtga |
| 2521 | aaagctattg tgtcaagaga cagtgttaat taaactggga aaatacaaaa gaactgtttt |
| 2581 | tttgaataat atgttagact gtatttatgt tgtttctaat aaaaataagt gttttcagca |
| 2641 | gaaaaaaaaa aaaaaaaaaa |
| SEQ ID NO: 20 | |
| NP 032633 Murine MMP-13 | |
| 1 | mhsailatff llswtpcwsl plpygddddd dlseedlvfa ehylksyyhp atlagilkks |
| 61 | tvtstvdrlr emqsffglev tgklddptld imrkprcgvp dvgeynvfpr tlkwsqtnlt |
| 121 | yrivnytpdm shsevekafr kafkvwsdvt plnftriydg tadimisfgt kehgdfypfd |
| 181 | gpsgllahaf ppgpnyggda hfdddetwts sskgynlfiv aahelghslg ldhskdpgal |
| 241 | mfpiytytgk shfmlpdddv qgiqflyqpg dedpnpkhpk tpekcdpals ldaitslrge |
| 301 | tmifkdrffw rlhpqqveae lfltksfwpe lpnhvdaaye hpsrdlmfif rgrkfwalng |
| 361 | ydilegyprk isdlgfpkev krlsaavhfe ntgktlffse nhvwsyddvn qtmdkdyprl |
| 421 | ieeefpgign kvdavyekng yiyffngpiq feysiwsnri vrvmptnsil wc |
| SEQ ID NO: 21 | |
| NM 008608 Murine MMP-14 | |
| 1 | ggagaaggga gggaccaaag gagagcagag agggcttcca actcagttcg ccgactaagc |
| 61 | agaagaaaga tcaaaaaacg gaaaagagaa gagcaaacag acatttccag gagcaattcc |
| 121 | ctcacctcca agccgaccgc gctctaggaa tccacattcc gttcctttag aagacaaagg |
| 181 | cgccccaaga gaggcggcgc gaccccaggg cgtgggcccc gccgcggagc ccgcaccgcc |
| 241 | cggcgccccg acgccgggga ccatgtctcc cgcccctcga ccctcccgca gcctcctgct |
| 301 | ccccctgctc acgcttggca cggcgctcgc ctccctcggc tgggcccaag gcagcaactt |
| 361 | cagccccgaa gcctggctgc agcagtatgg ctacctacct ccaggggacc tgcgtaccca |
| 421 | cacacaacgc tcaccccagt cactctcagc tgccattgcc gccatgcaaa agttctatgg |
| 481 | tttacaagtg acaggcaagg ctgatttggc aaccatgatg gccatgaggc gccctcgctg |
| 541 | tggtgttccg gataagtttg ggactgagat caaggccaat gttcggagga agcgctatgc |
| 601 | cattcagggc ctcaagtggc agcataatga gatcactttc tgcattcaga attacacccc |
| 661 | taaggtgggc gagtatgcca cattcgaggc cattcggaag gccttccgag tatgggagag |
| 721 | tgccacgcca ctgcgcttcc gagaagtgcc ctatgcctac atccgggagg gacatgagaa |
| 781 | gcaggctgac atcatgatct tgtttgctga gggtttccac ggcgacagta caccctttga |
| 841 | tggtgaagga gggttcctgg ctcatgccta cttcccaggc cccaatattg gaggggatac |
| 901 | ccactttgat tctgccgagc cctggactgt ccaaaatgag gatctaaatg ggaatgacat |
| 961 | cttcttggtg gctgtgcatg agttggggca tgccctaggc ctggaacatt ccaatgatcc |
| 1021 | ctccgccatc atggccccct tttaccagtg gatggacaca gagaacttcg tgttgcctga |
| 1081 | tgacgatcgc cgtggcatcc agcaacttta tggaagcaag tcagggtcac ccacaaagat |
| 1141 | gccccctcaa cccagaacta cctctcggcc ctctgtccca gataagccca aaaaccccgc |
| 1201 | ctatgggccc aacatctgtg acgggaactt tgacaccgtg gccatgctcc gaggagagat |
| 1261 | gtttgtcttc aaggagcgat ggttctggcg ggtgaggaat aaccaagtga tggatggata |
| 1321 | cccaatgccc attggccaat tctggagggg cctgcctgca tccatcaata ctgcctacga |
| 1381 | gaggaaggat ggcaaatttg tcttcttcaa aggagataag cactgggtgt ttgacgaagc |
| 1441 | ctccctggaa cccgggtacc ccaagcacat taaggagctg ggccgagggc tgcccacgga |
| 1501 | caagatcgat gcagctctct tctggatgcc caatgggaag acctacttct tccggggcaa |
| 1561 | taagtactac cggttcaatg aagaattcag ggcagtggac agcgagtacc ctaaaaacat |
| 1621 | caaagtctgg gaaggaatcc ctgaatctcc cagggggtca ttcatgggca gtgatgaagt |
| 1681 | cttcacatac ttctacaagg gaaacaaata ctggaagttc aacaaccaga agctgaaggt |
| 1741 | agagccaggg tatcccaagt cagctctgcg ggactggatg ggctgccctt cggggggccg |
| 1801 | gcccgatgag gggactgagg aggagacgga ggtgatcatc attgaggtgg atgaggaggg |
| 1861 | cagtggagct gtgagtgcgg ccgccgtggt cctgccggta ctactgctgc tcctggtact |
| 1921 | ggcagtgggc ctcgctgtct tcttcttcag acgccatggg acgcccaagc gactgcttta |
| 1981 | ctgccagcgt tcgctgctgg acaaggtctg acccccacca ctggcccacc cgcttctacc |
| 2041 | acaaggactt tgcctctgaa ggccagtggc tacaggtggt agcaggtggg ctgctctcac |
| 2101 | ccgtcctggg ctccctccct ccagcctccc ttctcagtcc ctaattggcc tctcccaccc |
| 2161 | tcaccccagc attgcttcat ccataagtgg gtcccttgag ggctgagcag aagacggttg |
| 2221 | gcctctggcc ctcaagggaa tctcacagct cggtgtgtgt tcagccctag ttgaatgttg |
| 2281 | tcaaggctct gcacttgaag gcaagaccct ctgaccttat aggcaacggc caaatggggc |
| 2341 | catctgcttc ttttccatcc ccctaactac ataccttaaa tctctgaact ctgacctcag |
| 2401 | gaggctctgg gcatatgagc cctatatgta ccaagtgtac ctagttggct gcctcccgcc |
| 2461 | actctgacta aaaggaatct taagagtgta cgtttggagg tggaaagatt gttcagttta |
| 2521 | ccctaaagac tttgataaga aagagaaaga aagaaagaaa gaaagaaaga aagaaagaaa |
| 2581 | gaaagaaaga aagaaag |
| SEQ ID NO: 22 | |
| NM 011594 Murine TIMP-2 | |
| 1 | mspaprpsrs lllplltlgt alaslgwaqg snfspeawlq qygylppgdl rthtqrspqs |
| 61 | lsaaiaamqk fyglqvtgka dlatmmamrr prcgvpdkfg teikanvrrk ryaiqglkwq |
| 121 | hneitfciqn ytpkvgeyat feairkafrv wesatplrfr evpyayireg hekqadimil |
| 181 | faegfhgdst pfdgeggfla hayfpgpnig gdthfdsaep wtvqnedlng ndiflvavhe |
| 241 | lghalglehs ndpsaimapf yqwmdtenfv lpdddrrgiq qlygsksgsp tkmppqprtt |
| 301 | srpsvpdkpk npaygpnicd gnfdtvamlr gemfvfkerw fwrvrnnqvm dgypmpigqf |
| 361 | wrglpasint ayerkdgkfv ffkgdkhwvf deaalepgyp khikelgrgl ptdkidaalf |
| 421 | wmpngktyff rgnkyyrfne efravdseyp knikvwegip esprgsfmgs devftyfykg |
| 481 | nkywkfnnqk lkvepgypks alrdwmgcps ggrpdegtee eteviiievd eegsgavsaa |
| 541 | avvlpvllll lvlavglavf ffrrhgtpkr llycqrslld kv |
| SEQ ID NO: 23 | |
| NM 011594 Murine TIMP-2 | |
| 1 | ccggcctgca ctggccgcca gccaccgaga ggaggagcag aggatcctcg gagcgcaata |
| 61 | aaacggcggc tcggcccgag cccgcagcaa acacagccat agaaggcagc ggaggagccg |
| 121 | agccgggctg cgctcgctcg ccgcccccca gcctctttct tctccgccgg gtgcactgcc |
| 181 | ctgcgccgtc ccctcgccgc tgcgcccctt gacaaagagg acagaaagtt tgcgcggggg |
| 241 | agcgggccag gtgaggaggg gcgtgcccgg cgccccagtc cgcgccccag cagccggacc |
| 301 | caggccccca gcgcgcccgc catgggcgcc gcggcccgca gcctccggct ggcgctcggc |
| 361 | ctcctgctgc tagccacgct gctgcgcccg gccgacgcct gcagctgctc cccggtgcac |
| 421 | ccgcaacagg cgttttgcaa tgcagacgta gtgatcagag ccaaagcagt gagcgagaag |
| 481 | gaggtggatt ccgggaatga catctatggc aaccccatca agaggattca gtatgagatc |
| 541 | aagcagataa agatgttcaa aggacctgac aaagacatcg agtttatcta cacggccccc |
| 601 | tcttcagcag tgtgcggggt ctcgctggac gttggaggaa agaaggagta tctaattgca |
| 661 | ggaaaggcag aaggagatgg caagatgcac attaccctct gtgacttcat tgtgccctgg |
| 721 | gacacgctta gcatcaccca gaagaagagc ctgaaccaca ggtaccagat gggctgtgag |
| 781 | tgcaagatca ctcgctgtcc catgatccct tgctacatct cctccccgga tgagtgcctc |
| 841 | tggatggact gggtcacaga gaagagcatc aatgggcacc aggccaagtt cttcgcctgc |
| 901 | atcaagagaa gtgatggttc ttgcgcgtgg taccgcgggg cggcaccccc caagcaagag |
| 961 | tttcttgaca tcgaggaccc gtaagaaggc tgacagagcc cctgtggcca attgaaaagc |
| 1021 | ctctgagggt ttagactggt ccagctttga catcccttcc tggaaacagc atgaataaaa |
| 1081 | catcaatcat ccaagtgggt tcacgctagt gtgattctgc cccctcccct attttcccta |
| 1141 | gacatggtag tgggtctgga gggacaggcg ggccaggttc cctgccatac cccttccctc |
| 1201 | tgccagcctg agcactgtgt gtctcagtct ttgatccttg ctacaggcag gagtggagca |
| 1261 | cagacttgtt accaggtctc tctggcactg tcacatgcag cagacaggca gcattaaggg |
| 1321 | taccctagct ctgttagggc agagcctggg aatgtgcatt ttgcagaaac tcttgaaggt |
| 1381 | tgttgtaaga ctgtgtagcc ggcctaccag gtccttttca tcctgagagt gacatgtccc |
| 1441 | tcgttttctg cagtggccac ctctctctct ggcccttgca aatgcttccc atccctcctg |
| 1501 | catctggtat ggactttcag gaccctggtc tccctcgggt ctaagaatca ccctccaacc |
| 1561 | agtggttcat ttttctagga gtcccagtca gccccatgaa tccacagact tcagcgaatg |
| 1621 | gaagccctcc ctgagccgtg tttctggctt caaccaagtc attgctgcct tcctctcccc |
| 1681 | tgtctctaca cacaccctca gtggggtctg tgaggtctca tgctgggggc agggatctgt |
| 1741 | ggtgaggggt gcttggcagt ccttgttgcc actctcaagc ttcccaagcc attcttcacc |
| 1801 | cctttccaag caagcttcaa gcatccaggc tgagcagcac ggctcggttt ggctctctgt |
| 1861 | cgcatcaggc cctgccgctg ttggggggcg ctaccagcac tccctctttt gcacaaactg |
| 1921 | atgatataaa aggccagtcc taggcaccta ggaaaagtct agtgaactct ccctgctaga |
| 1981 | tcagcggtca ttatgaccct gttgatttct gtgtcagtaa ctaagcacgc agcaggggag |
| 2041 | actttgcggt gggcagttct tcgtaccaag cccccccccc caaggatgct gtggcttgtt |
| 2101 | ttcagatcca tctcattttc ctaaaggtga attctcatgc atggctgaga gacatgtgta |
| 2161 | tgcagctctg cttctgtctc ttaatgtcgc ttaaggccct tatagggaac tggtatatct |
| 2221 | acttgctcct taaagcaatt ttctttctga tgccaatagg atgggggtta agacaggcag |
| 2281 | agctttatca ctaacaatat agacagccac tcttccttct gcctgcgtct taaaaaataa |
| 2341 | gctgtcccaa ggacacaaat gtatattatc catacatgca cgcatatgct cacacacaaa |
| 2401 | ctcagactct gaagtctggt agcctgtgaa tgttcctttt gtaaaatgct tccaaaagcc |
| 2461 | tctttgctcc aaccctgtcc taaccatcag aaaccccaaa gaaacggtta aggactcccc |
| 2521 | ctcagactct ccctacccaa gcccctacac caggacctgg ccagtccttt taagacagac |
| 2581 | tgggaggaca cacaggagtc agcctgcccc ttctgagggc attttcgtgt tgtgcagtga |
| 2641 | tgttcttcct tggatgctgg cctggaccag ccaacgagac cctgcagtct atcccgccct |
| 2701 | gcctgtttgc ttcctgtgcg gtggtatcaa tatgtaacag tgcctgtttc tgctgatttc |
| 2761 | atgacatgtt ctggtttgtt tctgatgttc gccgtgagcg ttcttgtgcc gtgttgatgc |
| 2821 | ctttcgtagc attagacttt gcacttttaa aaaaaaaaaa aacaaaaatg ttgaagcatc |
| 2881 | gaggaagcat cgaacccaga gtggaatgca tggtatggta gctggcctgg gacagaggga |
| 2941 | ccctttctca tcttcctttg agttctttga ctatctgctt ttccagcctc tcccgtcttt |
| 3001 | tgtatctggt tcaaattatt ataaaggaaa gaccctctga gtataccggt tctgaaagac |
| 3061 | ggcctttctg ttttccactc atgctggggt ttctagccac accaggcaga tgagaggaaa |
| 3121 | ccgagcgagc aaacgaacct ttgggacaaa gtgccagatg gcagctgagc aacagccact |
| 3181 | caaatgcctt cccagcaaac caattgcaat atatagttta aggtgttgtt ttacttctgt |
| 3241 | tatattctaa gccctgggcc tccctccctt actcccgtca tgccagcaac tcgcaatatt |
| 3301 | tcagatgacg tttacatggt agcaatttcc aaatcgctgc ctgatgcgta ttaagacata |
| 3361 | tccgtgggct tgctgcataa ctcaacgctt tgttgatttt gtttctgttt gaactcttgg |
| 3421 | ttgtaggggg ggaggggtgg aaccccatgt gcgtgctgga atatgaagtc tgagatgtac |
| 3481 | cccccaacac cccacgctgg cgatacgtga gagttgttga aagtcagcaa gccgagcgcg |
| 3541 | cctgatgctc tgtatcagtc tctactttta tttttatgag tttgctctgt caatggacaa |
| 3601 | taaaccatat tatcaaagag aaaaaaaaaa aaaaa |
| SEQ ID NO: 24 | |
| NP 035724 Murine TIMP-2 | |
| 1 | mgaaarslrl algllllatl lrpadacscs pvhpqqafcn advvirakav sekevdsgnd |
| 61 | iygnpikriq yeikqikmfk gpdkdiefiy tapssavcgv sldvggkkey liagkaegdg |
| 121 | kmhitlcdfi vpwdtlsitq kkslnhryqm gceckitrcp mipcyisspd eclwmdwvte |
| 181 | ksinghqakf facikrsdgs cawyrgaapp kqefldiedp |
| SEQ ID NO: 25 | |
| PCR Primer sequence Murine Flt3 Ligand Forward | |
| CATATCATGACAGTGCTGGCGCCAGCC | |
| SEQ ID NO: 26 | |
| PCR Primer sequence Murine Flt3 Ligand Reverse | |
| GTAAGGATCCTAGGGATGGGAGGGGAGG | |
| SEQ ID NO: 27 | |
| PCR Primer sequence MMP-13 Reverse | |
| GGCTCTGAATGGTTATGACATTCTG | |
| SEQ ID NO: 28 | |
| PCR Primer sequence MMP-13 Forward | |
| AGAGGGTCTTCCCCGTGTTCT | |
| SEQ ID NO: 29 | |
| PCR Primer sequence MMP-10 Reverse | |
| CGTGCTGACTGAATCAAAGGAC | |
| SEQ ID NO: 30 | |
| PCR Primer sequence MMP-10 Forward | |
| CCTGTGTTGTCTGTCTCTCCAAGA | |
| SEQ ID NO: 31 | |
| PCR Primer sequence MMP-9 Reverse | |
| GGAAACTCACACGCCAGAAGA | |
| SEQ ID NO: 32 | |
| PCR Primer sequence MMP-9 Forward | |
| AGCGTCATTCGCGTGGATA | |
| SEQ ID NO: 33 | |
| PCR Primer sequence TIMP-1 Reverse | |
| CTCAGAGTACGCCAGGGAAC | |
| SEQ ID NO: 34 | |
| PCR Primer sequence TIMP-1 Forward | |
| CATGGAAAGCCTCTGTGGAT | |
| SEQ ID NO: 35 | |
| PCR Primer sequence TIMP-2 Reverse | |
| CTCAGAGTACGCCAGGGAAC | |
| SEQ ID NO: 36 | |
| PCR Primer sequence TIMP-2 Forward | |
| GTCCATCCAGAGGCACTCAT | |
| SEQ ID NO: 37 | |
| PCR Primer sequence MMP-14 Reverse | |
| Reverse: CAATGGGCATTGGGTATCC | |
| SEQ ID NO: 38 | |
| PCR Primer sequence MMP-14 Forward | |
| AACTTTGACACCGTGGCCA | |
| SEQ ID NO: 39 | |
| PCR Primer sequence MMP-2 Reverse | |
| AAGGACCGGTTTATTTGGCG | |
| SEQ ID NO: 40 | |
| PCR Primer sequence MMP-2 Forward | |
| CTCCCCCGATGCTGATACTG | |
| SEQ ID NO: 41 | |
| PCR Primer sequence GAPDH Reverse | |
| GATCTCGCTCCTGGAAGATG | |
| SEQ ID NO: 42 | |
| PCR Primer sequence GAPDH Forward | |
| CAATGACCCCTTCATTGACC | |
| SEQ ID NO: 43 | |
| NP 003245 Human TIMP-1 | |
| 1 | mapfeplasg illllwliap sractcvpph pqtafcnsdl virakfvgtp evnqttlyqr |
| 61 | yeikmtkmyk grqalgdaad irfvytpame svcgyfhrsh nrseefliag klqdgllhit |
| 121 | tcgfvapwns lslaqrrgft ktytvgceec tvfpclsipc klqsgthclw tdgllqgsek |
| 181 | gfqsrhlacl prepglctwq slrsqia |
| SEQ ID NO: 44 | |
| NM 003254 Human TIMP-1 | |
| 1 | tttcgtcggc ccgccccttg gcttctgcac tgatggtggg tggatgagta atgcatccag |
| 61 | gaagcctgga ggcctgtggt ttccgcaccc gctgccaccc ccgcccctag cgtggacatt |
| 121 | tatcctctag cgctcaggcc ctgccgccat cgccgcagat ccagcgccca gagagacacc |
| 181 | agagaaccca ccatggcccc ctttgagccc ctggcttctg gcatcctgtt gttgctgtgg |
| 241 | ctgatagccc ccagcagggc ctgcacctgt gtcccacccc acccacagac ggccttctgc |
| 301 | aattccgacc tcgtcatcag ggccaagttc gtggggacac cagaagtcaa ccagaccacc |
| 361 | ttataccagc gttatgagat caagatgacc aagatgtata aagggttcca agccttaggg |
| 421 | gatgccgctg acatccggtt cgtctacacc cccgccatgg agagtgtctg cggatacttc |
| 481 | cacaggtccc acaaccgcag cgaggagttt ctcattgctg gaaaactgca ggatggactc |
| 541 | ttgcacatca ctacctgcag ttttgtggct ccctggaaca gcctgagctt agctcagcgc |
| 601 | cggggcttca ccaagaccta cactgttggc tgtgaggaat gcacagtgtt tccctgttta |
| 661 | tccatcccct gcaaactgca gagtggcact cattgcttgt ggacggacca gctcctccaa |
| 721 | ggctctgaaa agggcttcca gtcccgtcac cttgcctgcc tgcctcggga gccagggctg |
| 781 | tgcacctggc agtccctgcg gtcccagata gcctgaatcc tgcccggagt ggaagctgaa |
| 841 | gcctgcacag tgtccaccct gttcccactc ccatctttct tccggacaat gaaataaaga |
| 901 | gttaccaccc agcagaaaaa aaaaaaaaaa a |
| SEQ ID NO: 45 | |
| NP 001037849 Murine TIMP-1 | |
| 1 | mmapfaslas gillllslia sskacscapp hpqtafcnsd lvirakfmgs peinettlyq |
| 61 | rykikmtkml kgfkavgnaa diryaytpvm eslcgyahks qnrseeflit grlrngnlhi |
| 121 | sacsflvpwr tlspaqqraf sktysagcgv ctvfpclsip cklesdthcl wtdqvlvgse |
| 181 | dyqsrhfacl prnpglctwr slgar |
| SEQ ID NO: 46 | |
| NM 001044384 Murine TIMP-1 | |
| 1 | aggctttgac tccagcggtg ggtggatgag taatgcgtcc aggaagcctg agggcagtga |
| 61 | tttccccgcc aactccgccc ttcgcatgga catttattct ccactgtgca gcccctgccg |
| 121 | ccatcatcgc agatcggggc tcctagagac acaccagagc agataccatg atggccccct |
| 181 | ttgcatctct ggcatctggc atcctcttgt tgctatcact gatagcttcc agtaaggcct |
| 241 | gtagctgtgc cccaccccac ccacagacag ccttctgcaa ctcggacctg gtcataaggg |
| 301 | ctaaattcat gggttcccca gaaatcaacg agaccacctt ataccagcgt tataagatca |
| 361 | agatgactaa gatgctaaaa ggattcaagg ctgtgggaaa tgccgcagat atccggtacg |
| 421 | cctacacccc agtcatggaa agcctctgtg gatatgccca caagtcccag aaccgcagtg |
| 481 | aagagtttct catcacgggc cgcctaagga acggaaattt gcacatcagt gcctgcagct |
| 541 | tcttggttcc ctggcgtact ctgagccctg ctcagcaaag agctttctca aagacctata |
| 601 | gtgctggctg tggggtgtgc acagtgtttc cctgtttatc tatcccttgc aaactggaga |
| 661 | gtgacactca ctgtttgtgg acggatcagg tcctcgtggg ctctgaggac taccagagcc |
| 721 | gtcactttgc ttgcctgcca cggaatccag gcttgtgcac ctggagatcc cttggggccc |
| 781 | gatgacctga agccttcccc caggaaaaac tgaagcctga acactgtcta cttttcctcc |
| 841 | atctttcttt ctcttagatg gtgaaataaa gaactatcag acagcagcaa aaaaaaaaaa |
| 901 | aaaaa |

What is claimed:

1. A method for treating fibrosis in a mammal which comprises administering to the mammal an effective amount for treating fibrosis of Flt3 ligand.

2. The method of claim 1, wherein the Flt3 ligand is administered as a cell expressing and secreting the Flt3 ligand.

3. The method of claim 1, wherein the Flt3 ligand is administered as a plasmid comprising the nucleic acid sequence of Flt3 ligand, wherein
the plasmid is administered in an amount that is effective for yielding expression of Flt3 ligand in said patient; and wherein
the Flt3 ligand is expressed in an amount that is effective for the treatment of fibrosis.

4. The method of claim 1, wherein the fibrosis is hepatic or pulmonary fibrosis.

5. The method of claim 1, wherein the cell expresses Flt3 ligand as a mutant or variant of Flt3 ligand.

6. The method of claim 1, wherein the mammal is afflicted with fibrosis in the liver, pancreas, lung, heart, nervous system, skin, kidneys, bone marrow, lymph nodes, endomyocardium, or retroperitoneum.

7. The method of claim 1, further comprising administering a cytokine or growth factor.

8. The method of claim 1, wherein the cell is administered parenterally.

9. The method of claim 1, wherein the cell is administered orally.

10. The method of claim 1, wherein the cell is administered by inhalation.

11. The method of claim 2, wherein the patient is a human.

12. The method of claim 8, wherein the cell is administered by subcutaneous injection, intravenous injection, intramuscular injection, intracisternal injection or infusion.

13. A method for treating fibrosis in a patient in need thereof, comprising administering the patient an effective amount of dendritic cells.

14. The method of claim 13, wherein the dendritic cells are expanded from CD34⁺ progenitor cells treated with Flt3 ligand.

15. The method of claim 13, wherein the dendritic cells are expanded from CD34⁺ progenitor cells treated with a cytokine or growth factor.

16. The method of claim 13, wherein the patient is a human.

17. The method of claim 13, wherein the patient is afflicted with fibrosis in the liver, pancreas, lung, heart, nervous system, skin, kidneys, bone marrow, lymph nodes, endomyocardium or retroperitoneum.

18. The method of claim 13, wherein the fibrosis is hepatic or pulmonary fibrosis.

19. The method of claim 13, wherein the cell is administered parenterally.

20. The method of claim 13, wherein the cell is administered orally.

21. The method of claim 13, wherein the cell is administered by inhalation.

22. The method of claim 13, further comprising administration of one or more additional growth factors or cytokines.

23. The method of claim 19, wherein the cell is administered by subcutaneous injection, intravenous injection, intramuscular injection, intracisternal injection or infusion.

24. A pharmaceutical formulation comprising Flt3 ligand and a pharmaceutical carrier.

25. The pharmaceutical formulation of claim 24, further comprising another cytokine or growth factor.

26. A method for the treatment of fibrosis which comprises administering to a patient in need of such treatment an effective amount for treating fibrosis of the pharmaceutical formulation according to claim 24.

27. A method for the treatment of fibrosis which comprises administering to a patient in need of such treatment an effective amount of the pharmaceutical formulation according to claim 24, and wherein the fibrosis is afflicting an organ or tissue selected from liver, pancreas, lung, heart, nervous system, skin, kidneys, bone marrow, lymph nodes, endomyocardium, and retroperitoneum.

28. A method of treatment, comprising administering to a patient in need of such treatment an effective amount for treating said disease or condition of the pharmaceutical formulation according to claim 24, wherein the disease or condition is a member selected from the group consisting of cirrhosis, diffuse parenchymal lung disease, post-vasectomy pain syndrome, tuberculosis, sickle-cell anemia, rheumatoid arthritis, progressive massive fibrosis, idiopathic pulmonary fibrosis, injection fibrosis, renal fibrosis, myelofibrosis, cardiac fibrosis, liver fibrosis, pancreatic fibrosis, skin fibrosis, scleroderma, intestinal fibrosis or strictures, and mediastinal fibrosis.

29. A pharmaceutical formulation comprising a dendritic cell expanded from CD34⁺ progenitor cells or bone marrow cells treated with Flt3 ligand.

30. A method for increasing the amount of dendritic cells in a mammal, comprising administering to the mammal, an effective amount of Flt3 ligand.

31. The method of claim 30, further comprising administering to the mammal an additional cytokine or growth factor.

32. The method of claim 30, wherein Flt3 ligand is administered parenterally.


SEQUENCE LISTING (46 sequences)

SEQ ID NO: 1 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 2 - DNA, 3848 bp, Homo sapiens
acctgcagcgcgaggcgcgccgctccaggcggcatcgcagggctgggccggcgcggcctggggaccccgggctccggaggccatgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgacgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagctgtctgcgtttactcttgttttcaaagggacttttgtaaaatcaaatcatcctgtcacaaggcaggaggagctgataatgaactttattggagcattgatctgcatccaaggccttctcaggctggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacagaattgcccattattgaattatttacatggtataattagggcaagtcttaactggagttcacgaaccccctgaaattgtgcacccatagccacctacacattccttccagagcacgtgtgcttttaccccaagatacaaggaatgtgtaggcagctatggttgtcacagcctaagatttctgcaacaacaggggttgtattgggggaagtttataatgaataggtgttctaccataaagagtaatacatcacctagacactttggcggccttcccagactcagggccagtcagaagtaacatggaggattagtattttcaataaagttactcttgtccccacaaaaaaa

SEQ ID NO: 3 - protein, 1000 aa, Mus musculus
MRALAQRSDRRLLLLVVLSVMILETVTNQDLPVIKCVLISHENNGSSAGKPSSYRMVRGSPEDLQCAPRRQSEGTVYEAATVEVAESGSITLQVQLATPGDLSCLWVFKHSSLGCQPHFDLQNRGIVSMAILNVTETQAGEYLLHIQSEAANYTVLFTVNVRDTQLYVLRRPYFRKMENQDALLCISEGVPEPTVEWVLCSSHRESCKEEGPAVVRKEEKVLHELFGTDIRCCARNALGRECTKLFTIDLNQAPQSTLPQLFLKVGEPLWIRCKAIHVNHGFGLTWELEDKALEEGSYFEMSTYSTNRTMIRILLAFVSSVGRNDTGYYTCSSSKHPSQSALVTILEKGFINATSSQEEYEIDPYEKFCFSVRFKAYPRIRCTWIFSQASFPCEQRGLEDGYSISKFCDHKNKPGEYIFYAENDDAQFTKMFTLNIRKKPQVLANASASQASCSSDGYPLPSWTWKKCSDKSPNCTEEIPEGVWNKKANRKVFGQWVSSSTLNMSEAGKGLLVKCCAYNSMGTSCETIFLNSPGPFPFIQDNISFYATIGLCLPFIVVLIVLICHKYKKQFRYESQLQMIQVTGPLDNEYFYVDFRDYEYDLKWEFPRENLEFGKVLGSGAFGRVMNATAYGISKTGVSIQVAVKMLKEKADSCEKEALMSELKMMTHLGHHDNIVNLLGACTLSGPVYLIFEYCCYGDLLNYLRSKREKFHRTWTEIFKEHNFSFYPTFQAHSNSSMPGSREVQLHPPLDQLSGFNGNLIHSEDEIEYENQKRLAEEEEEDLNVLTFEDLLCFAYQVAKGMEFLEFKSCVHRDLAARNVLVTHGKVVKICDFGLARDILSDSSYVVRGNARLPVKWMAPESLFEGIYTIKSDVWSYGILLWEIFSLGVNPYPGIPVDANFYKLIQSGFKMEQPFYATEGIYFVMQSCWAFDSRKRPSFPNLTSFLGCQLAEAEEAMYQNMGGNVPEHPSIYQNRRPLSREAGSEPPSPQAQVKIHGERS

SEQ ID NO: 4 - DNA, 3664 bp, Mus musculus
gggcacgtgggatcggctgcagcactgcgccagttcagcccgcctagcagcgagcggccgcggcctctggagagaggttcctccccctctgctctgcaccagtccgagggaatctgtggtcagtgacgcgcatccttcagcgagccacctgcagcccggggcgcgccgctgggaccgcatcacaggctgggccggcggcctggctaccgcgcgctccggaggccatgcgggcgttggcgcagcgcagcgaccggcggctgctgctgcttgttgttttgtcagtaatgattcttgagaccgttacaaaccaagacctgcctgtgatcaagtgtgttttaatcagtcatgagaacaatggctcatcagcgggaaagccatcatcgtaccgaatggtgcgaggatccccagaagacctccagtgtgccccgaggcgccagagtgaagggacggtatatgaagcggccaccgtggaggtggccgagtctgggtccatcaccctgcaagtgcagctcgccaccccaggggacctttcctgcctctgggtctttaagcacagctccctgggctgccagccgcactttgatttacaaaacagaggaatcgtttccatggccatcttgaacgtgacagagacccaggcaggagaatacctactccatattcagagcgaagccgccaactacacagtactgttcacagtgaatgtaagagatacacagctgtacgtgctaagaagaccttactttaggaagatggaaaaccaggacgcactgctctgcatctccgagggtgttccagagcccactgtggagtgggtgctctgcagctcccacagggaaagctgtaaagaagaaggccctgctgttgtcagaaaggaggaaaaggtacttcatgagttgttcggaacagacatcagatgctgtgctagaaatgcactgggccgcgaatgcaccaagctgttcaccatagatctaaaccaggctcctcagagcacactgccccagttattcctgaaagtgggggaacccttgtggatcaggtgtaaggccatccatgtgaaccatggattcgggctcacctgggagctggaagacaaagccctggaggagggcagctactttgagatgagtacctactccacaaacaggaccatgattcggattctcttggcctttgtgtcttccgtgggaaggaacgacaccggatattacacctgctcttcctcaaagcaccccagccagtcagcgttggtgaccatcctagaaaaagggtttataaacgctaccagctcgcaagaagagtatgaaattgacccgtacgaaaagttctgcttctcagtcaggtttaaagcgtacccacgaatccgatgcacgtggatcttctctcaagcctcatttccttgtgaacagagaggcctggaggatgggtacagcatatctaaattttgcgatcataagaacaagccaggagagtacatattctatgcagaaaatgatgacgcccagttcaccaaaatgttcacgctgaatataagaaagaaacctcaagtgctagcaaatgcctcagccagccaggcgtcctgttcctctgatggctacccgctaccctcttggacctggaagaagtgttcggacaaatctcccaattgcacggaggaaatcccagaaggagtttggaataaaaaggctaacagaaaagtgtttggccagtgggtgtcgagcagtactctaaatatgagtgaggccgggaaagggcttctggtcaaatgctgtgcgtacaattctatgggcacgtcttgcgaaaccatctttttaaactcaccaggccccttccctttcatccaagacaacatctccttctatgcgaccattgggctctgtctccccttcattgttgttctcattgtgttgatctgccacaaatacaaaaagcaatttaggtacgagagtcagctgcagatgatccaggtgactggccccctggataacgagtacttctacgttgacttcagggactatgaatatgaccttaagtgggagttcccgagagagaacttagagtttgggaaggtcctggggtctggcgctttcgggagggtgatgaacgccacggcctatggcattagtaaaacgggagtctcaattcaggtggcggtgaagatgctaaaagagaaagctgacagctgtgaaaaagaagctctcatgtcggagctcaaaatgatgacccacctgggacaccatgacaacatcgtgaatctgctgggggcatgcacactgtcagggccagtgtacttgatttttgaatattgttgctatggtgacctcctcaactacctaagaagtaaaagagagaagtttcacaggacatggacagagatttttaaggaacataatttcagtttttaccctactttccaggcacattcaaattccagcatgcctggttcacgagaagttcagttacacccgcccttggatcagctctcagggttcaatgggaatttaattcattctgaagatgagattgaatatgaaaaccagaagaggctggcagaagaagaggaggaagatttgaacgtgctgacgtttgaagacctcctttgctttgcgtaccaagtggccaaaggcatggaattcctggagttcaagtcgtgtgtccacagagacctggcagccaggaatgtgttggtcacccacgggaaggtggtgaagatctgtgactttggactggcccgagacatcctgagcgactccagctacgtcgtcaggggcaacgcacggctgccggtgaagtggatggcacctgagagcttatttgaagggatctacacaatcaagagtgacgtctggtcctacggcatccttctctgggagatattttcactgggtgtgaacccttaccctggcattcctgtcgacgctaacttctataaactgattcagagtggatttaaaatggagcagccattctatgccacagaagggatatactttgtaatgcaatcctgctgggcttttgactcaaggaagcggccatccttccccaacctgacttcatttttaggatgtcagctggcagaggcagaagaagcgatgtatcagaacatgggtggcaacgtcccagaacatccatccatctaccaaaacaggcggcccctcagcagagaggcaggctcagagccgccatcgccacaggcccaggtgaagattcacggagaaagaagttagcgaggaggccttggaccccgccaccctagcaggctgtagaccacagagccaagattagcctcgcctctgaggaagcgccctacaggccgttgcttcgctggacttttctctagatgctgtctgccattactccaaagtgacttctataaaatcaaacctctcctcgcacaggtgggagagccaataatgagacttgttggtgagcccgcctaccctggggggcctttccaggccccccaggcttgaggggaaagccatgtatctgaaatatagtatattcttgtaaatacgtgaaacaaaccaaacccgttttttgctaagggaaagctaaatatgatttttaaaaatctatgttttaaaatactatgtaactttttcatctatttagtgatatattttatggatggaaataaactttctactgtagaaaaaaaaaa

SEQ ID NO: 5 - DNA, 1973 bp, Homo sapiens
gggatattggagtagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcggctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgtttt

SEQ ID NO: 6 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 7 - DNA, 2387 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccaggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagcggctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttggcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaacctttaaaaaaaaaaaaaaaaaaaaaaaaaaaaaaaaaaaaaaaaaaaaaaaaaaaa

SEQ ID NO: 8 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 9 - DNA, 1743 bp, Homo sapiens
aaagaaggtaagggcagtgagaatgatgcatcttgcattccttgtgctgttgtgtctgccagtctgctctgcctatcctctgagtggggcagcaaaagaggaggactccaacaaggatcttgcccagcaatacctagaaaagtactacaacctcgaaaaggatgtgaaacagtttagaagaaaggacagtaatctcattgttaaaaaaatccaaggaatgcagaagttccttgggttggaggtgacagggaagctagacactgacactctggaggtgatgcgcaagcccaggtgtggagttcctgacgttggtcacttcagctcctttcctggcatgccgaagtggaggaaaacccaccttacatacaggattgtgaattatacaccagatttgccaagagatgctgttgattctgccattgagaaagctctgaaagtctgggaagaggtgactccactcacattctccaggctgtatgaaggagaggctgatataatgatctctttcgcagttaaagaacatggagacttttactcttttgatggcccaggacacagtttggctcatgcctacccacctggacctgggctttatggagatattcactttgatgatgatgaaaaatggacagaagatgcatcaggcaccaatttattcctcgttgctgctcatgaacttggccactccctggggctctttcactcagccaacactgaagctttgatgtacccactctacaactcattcacagagctcgcccagttccgcctttcgcaagatgatgtgaatggcattcagtctctctacggacctccccctgcctctactgaggaacccctggtgcccacaaaatctgttccttcgggatctgagatgccagccaagtgtgatcctgctttgtccttcgatgccatcagcactctgaggggagaatatctgttctttaaagacagatatttttggcgaagatcccactggaaccctgaacctgaatttcatttgatttctgcattttggccctctcttccatcatatttggatgctgcatatgaagttaacagcagggacaccgtttttatttttaaaggaaatgagttctgggccatcagaggaaatgaggtacaagcaggttatccaagaggcatccataccctgggttttcctccaaccataaggaaaattgatgcagctgtttctgacaaggaaaagaagaaaacatacttctttgcagcggacaaatactggagatttgatgaaaatagccagtccatggagcaaggcttccctagactaatagctgatgactttccaggagttgagcctaaggttgatgctgtattacaggcatttggatttttctacttcttcagtggatcatcacagtttgagtttgaccccaatgccaggatggtgacacacatattaaagagtaacagctggttacattgctaggcgagatagggggaagacagatatgggtgtttttaataaatctaataattattcatctaatgtattatgagccaaaatggttaatttttcctgcatgttctgtgactgaagaagatgagccttgcagatatctgcatgtgtcatgaagaatgtttctggaattcttcacttgcttttgaattgcactgaacagaattaagaaatactcatgtgcaataggtgagagaatgtattttcatagatgtgttattacttcctcaataaaaagttttattttgggcctgttcctt

SEQ ID NO: 10 - protein, 476 aa, Homo sapiens
MMHLAFLVLLCLPVCSAYPLSGAAKEEDSNKDLAQQYLEKYYNLEKDVKQFRRKDSNLIVKKIQGMQKFLGLEVTGKLDTDTLEVMRKPRCGVPDVGHFSSFPGMPKWRKTHLTYRIVNYTPDLPRDAVDSAIEKALKVWEEVTPLTFSRLYEGEADIMISFAVKEHGDFYSFDGPGHSLAHAYPPGPGLYGDIHFDDDEKWTEDASGTNLFLVAAHELGHSLGLFHSANTEALMYPLYNSFTELAQFRLSQDDVNGIQSLYGPPPASTEEPLVPTKSVPSGSEMPAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 11 - DNA, 3558 bp, Homo sapiens
cagaccccagttcgccgactaagcagaagaaagatcaaaaaccggaaaagaggagaagagcaaacaggcactttgaggaacaatcccctttaactccaagccgacagcggtctaggaattcaagttcagtgcctaccgaagacaaaggcgccccgagggagtggcggtgcgaccccagggcgtgggcccggccgcggagcccacactgcccggctgacccggtggtctcggaccatgtctcccgccccaagacccccccgttgtctcctgctccccctgctcacgctcggcaccgcgctcgcctccctcggctcggcccaaagcagcagcttcagccccgaagcctggctacagcaatatggctacctgcctcccggggacctacgtacccacacacagcgctcaccccagtcactctcagcggccatcgctgccatgcagaagttttacggcttgcaagtaacaggcaaagctgatgcagacaccatgaaggccatgaggcgcccccgatgtggtgttccagacaagtttggggctgagatcaaggccaatgttcgaaggaagcgctacgccatccagggtctcaaatggcaacataatgaaatcactttctgcatccagaattacacccccaaggtgggcgagtatgccacatacgaggccattcgcaaggcgttccgcgtgtgggagagtgccacaccactgcgcttccgcgaggtgccctatgcctacatccgtgagggccatgagaagcaggccgacatcatgatcttctttgccgagggcttccatggcgacagcacgcccttcgatggtgagggcggcttcctggcccatgcctacttcccaggccccaacattggaggagacacccactttgactctgccgagccttggactgtcaggaatgaggatctgaatggaaatgacatcttcctggtggctgtgcacgagctgggccatgccctggggctcgagcattccagtgacccctcggccatcatggcacccttttaccagtggatggacacggagaattttgtgctgcccgatgatgaccgccggggcatccagcaactttatgggggtgagtcagggttccccaccaagatgccccctcaacccaggactacctcccggccttctgttcctgataaacccaaaaaccccacctatgggcccaacatctgtgacgggaactttgacaccgtggccatgctccgaggggagatgtttgtcttcaaggagcgctggttctggcgggtgaggaataaccaagtgatggatggatacccaatgcccattggccagttctggcggggcctgcctgcgtccatcaacactgcctacgagaggaaggatggcaaattcgtcttcttcaaaggagacaagcattgggtgtttgatgaggcgtccctggaacctggctaccccaagcacattaaggagctgggccgagggctgcctaccgacaagattgatgctgctctcttctggatgcccaatggaaagacctacttcttccgtggaaacaagtactaccgtttcaacgaagagctcagggcagtggatagcgagtaccccaagaacatcaaagtctgggaagggatccctgagtctcccagagggtcattcatgggcagcgatgaagtcttcacttacttctacaaggggaacaaatactggaaattcaacaaccagaagctgaaggtagaaccgggctaccccaagtcagccctgagggactggatgggctgcccatcgggaggccggccggatgaggggactgaggaggagacggaggtgatcatcattgaggtggacgaggagggcggcggggcggtgagcgcggctgccgtggtgctgcccgtgctgctgctgctcctggtgctggcggtgggccttgcagtcttcttcttcagacgccatgggacccccaggcgactgctctactgccagcgttccctgctggacaaggtctgacgcccaccgccggcccgcccactcctaccacaaggactttgcctctgaaggccagtggcagcaggtggtggtgggtgggctgctcccatcgtcccgagccccctccccgcagcctccttgcttctctctgtcccctggctggcctccttcaccctgaccgcctccctccctcctgccccggcattgcatcttccctagataggtcccctgagggctgagtgggagggcggccctttccagcctctgcccctcaggggaaccctgtagctttgtgtctgtccagccccatctgaatgtgttgggggctctgcacttgaaggcaggaccctcagacctcgctggtaaaggtcaaatggggtcatctgctccttttccatcccctgacataccttaacctctgaactctgacctcaggaggctctgggcactccagccctgaaagccccaggtgtacccaattggcagcctctcactactctttctggctaaaaggaatctaatcttgttgagggtagagaccctgagacagtgtgagggggtggggactgccaagccaccctaagaccttgggaggaaaactcagagagggtcttcgttgctcagtcagtcaagttcctcggagatctgcctctgcctcacctaccccagggaacttccaaggaaggagcctgagccactggggactaagtgggcagaagaaacccttggcagccctgtgcctctcgaatgttagccttggatggggctttcacagttagaagagctgaaaccaggggtgcagctgtcaggtagggtggggccggtgggagaggcccgggtcagagccctgggggtgagcctgaaggccacagagaaagaaccttgcccaaactcaggcagctggggctgaggcccaaaggcagaacagccagagggggcaggaggggaccaaaaaggaaaatgaggacgtgcagcagcattggaaggctggggccgggcaggccaggccaagccaagcagggggccacagggtgggctgtggagctctcaggaagggccctgaggaaggcacacttgctcctgttggtccctgtccttgctgcccaggcagcgtggaggggaagggtagggcagccagagaaaggagcagagaaggcacacaaacgaggaatgaggggcttcacgagaggccacagggcctggctggccacgctgtcccggcctgctcaccatctcagtgaggggcaggagctggggctcgcttaggctgggtccacgcttccctggtgccagcacccctcaagcctgtctcaccagtggcctgccctctcgctcccccacccagcccacccattgaagtctccttgggccaccaaaggtggtggccatggtaccggggacttgggagagtgagacccagtggagggagcaagaggagagggatgtcgggggggtggggcacggggtaggggaaatggggtgaacggtgctggcagttcggctagatttctgtcttgtttgtttttttgttttgtttaatgtatatttttattataattattatatatgaattccaaaaaaaaaaaaaaaaaaaaa

SEQ ID NO: 12 - protein, 582 aa, Homo sapiens
MSPAPRPPRCLLLPLLTLGTALASLGSAQSSSFSPEAWLQQYGYLPPGDLRTHTQRSPQSLSAAIAAMQKFYGLQVTGKADADTMKAMRRPRCGVPDKFGAEIKANVRRKRYAIQGLKWQHNEITFCIQNYTPKVGEYATYEAIRKAFRVWESATPLRFREVPYAYIREGHEKQADIMIFFAEGFHGDSTPFDGEGGFLAHAYFPGPNIGGDTHFDSAEPWTVRNEDLNGNDIFLVAVHELGHALGLEHSSDPSAIMAPFYQWMDTENFVLPDDDRRGIQQLYGGESGFPTKMPPQPRTTSRPSVPDKPKNPTYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSGGRPDEGTEEETEVIIIEVDEEGGGAVSAAAVVLPVLLLLLVLAVGLAVFFFRRHGTPRRLLYCQRSLLDKV

SEQ ID NO: 13 - DNA, 3670 bp, Homo sapiens
cgcagcaaacacatccgtagaaggcagcgcggccgccgagaaccgcagcgccgctcgcccgccgccccccaccccgccgccccgcccggcgaattgcgccccgcgcccctcccctcgcgcccccgagacaaagaggagagaaagtttgcgcggccgagcggggcaggtgaggagggtgagccgcgcgggaggggcccgcctcggccccggctcagcccccgcccgcgcccccagcccgccgccgcgagcagcgcccggaccccccagcggcggcccccgcccgcccagccccccggcccgccatgggcgccgcggcccgcaccctgcggctggcgctcggcctcctgctgctggcgacgctgcttcgcccggccgacgcctgcagctgctccccggtgcacccgcaacaggcgttttgcaatgcagatgtagtgatcagggccaaagcggtcagtgagaaggaagtggactctggaaacgacatttatggcaaccctatcaagaggatccagtatgagatcaagcagataaagatgttcaaagggcctgagaaggatatagagtttatctacacggccccctcctcggcagtgtgtggggtctcgctggacgttggaggaaagaaggaatatctcattgcaggaaaggccgagggggacggcaagatgcacatcaccctctgtgacttcatcgtgccctgggacaccctgagcaccacccagaagaagagcctgaaccacaggtaccagatgggctgcgagtgcaagatcacgcgctgccccatgatcccgtgctacatctcctccccggacgagtgcctctggatggactgggtcacagagaagaacatcaacgggcaccaggccaagttcttcgcctgcatcaagagaagtgacggctcctgtgcgtggtaccgcggcgcggcgccccccaagcaggagtttctcgacatcgaggacccataagcaggcctccaacgcccctgtggccaactgcaaaaaaagcctccaagggtttcgactggtccagctctgacatcccttcctggaaacagcatgaataaaacactcatcccatgggtccaaattaatatgattctgctccccccttctccttttagacatggttgtgggtctggagggagacgtgggtccaaggtcctcatcccatcctccctctgccaggcactatgtgtctggggcttcgatccttgggtgcaggcagggctgggacacgcggcttccctcccagtccctgccttggcaccgtcacagatgccaagcaggcagcacttagggatctcccagctgggttagggcagggcctggaaatgtgcattttgcagaaacttttgagggtcgttgcaagactgtgtagcaggcctaccaggtccctttcatcttgagagggacatggcccttgttttctgcagcttccacgcctctgcactccctgcccctggcaagtgctcccatcgccccggtgcccaccatgagctcccagcacctgactccccccacatccaagggcagcctggaaccagtggctagttcttgaaggagccccatcaatcctattaatcctcagaattccagtgggagcctccctctgagccttgtagaaatgggagcgagaaaccccagctgagctgcgttccagcctcagctgagtctttttggtctgcacccacccccccacccccccccccccgcccacatgctccccagcttgcaggaggaatcggtgaggtcctgtcctgaggctgctgtccggggccggtggctgccctcaaggtcccttccctagctgctgcggttgccattgcttcttgcctgttctggcatcaggcacctggattgagttgcacagctttgctttatccgggcttgtgtgcagggcccggctgggctccccatctgcacatcctgaggacagaaaaagctgggtcttgctgtgccctcccaggcttagtgttccctccctcaaagactgacagccatcgttctgcacggggctttctgcatgtgacgccagctaagcatagtaagaagtccagcctaggaagggaaggattttggaggtaggtggctttggtgacacactcacttctttctcagcctccaggacactatggcctgttttaagagacatcttatttttctaaaggtgaattctcagatgataggtgaacctgagttgcagatataccaacttctgcttgtatttcttaaatgacaaagattacctagctaagaaacttcctagggaactagggaacctatgtgttccctcagtgtggtttcctgaagccagtgatatgggggttaggataggaagaactttctcggtaatgataaggagaatctcttgtttcctcccacctgtgttgtaaagataaactgacgatatacaggcacattatgtaaacatacacacgcaatgaaaccgaagcttggcggcctgggcgtggtcttgcaaaatgcttccaaagccaccttagcctgttctattcagcggcaaccccaaagcacctgttaagactcctgacccccaagtggcatgcagcccccatgcccaccgggacctggtcagcacagatcttgatgacttccctttctagggcagactgggagggtatccaggaatcggcccctgccccacgggcgttttcatgctgtacagtgacctaaagttggtaagatgtcataatggaccagtccatgtgatttcagtatatacaactccaccagacccctccaacccatataacaccccacccctgttcgcttcctgtatggtgatatcatatgtaacatttactcctgtttctgctgattgtttttttaatgttttggtttgtttttgacatcagctgtaatcattcctgtgctgtgttttttattacccttggtaggtattagacttgcacttttttaaaaaaaggtttctgcatcgtggaagcatttgacccagagtggaacgcgtggcctatgcaggtggattccttcaggtctttcctttggttctttgagcatctttgctttcattcgtctcccgtctttggttctccagttcaaattattgcaaagtaaaggatctttgagtaggttcggtctgaaaggtgtggcctttatatttgatccacacacgttggtcttttaaccgtgctgagcagaaaacaaaacaggttaagaagagccgggtggcagctgacagaggaagccgctcaaataccttcacaataaatagtggcaatatatatatagtttaagaaggctctccatttggcatcgtttaatttatatgttatgttctaagcacagctctcttctcctattttcatcctgcaagcaactcaaaatatttaaaataaagtttacattgtagttattttcaaatctttgcttgataagtattaagaaatattggacttgctgccgtaatttaaagctctgttgattttgtttccgtttggatttttgggggaggggagcactgtgtttatgctggaatatgaagtctgagaccttccggtgctgggaacacacaagagttgttgaaagttgacaagcagactgcgcatgtctctgatgctttgtatcattcttgagcaatcgctcggtccgtggacaataaacagtattatcaaagagaaaaaaaaaaaaaaaaa

SEQ ID NO: 14 - protein, 220 aa, Homo sapiens
MGAAARTLRLALGLLLLATLLRPADACSCSPVHPQQAFCNADVVIRAKAVSEKEVDSGNDIYGNPIKRIQYEIKQIKMFKGPEKDIEFIYTAPSSAVCGVSLDVGGKKEYLIAGKAEGDGKMHITLCDFIVPWDTLSTTQKKSLNHRYQMGCECKITRCPMIPCYISSPDECLWMDWVTEKNINGHQAKFFACIKRSDGSCAWYRGAAPPKQEFLDIEDP

SEQ ID NO: 15 - DNA, 3185 bp, Mus musculus
ctcaccatgagtccctggcagcccctgctcctggctctcctggctttcggctgcagctctgctgccccttaccagcgccagccgacttttgtggtcttccccaaagacctgaaaacctccaacctcacggacacccagctggcagaggcatacttgtaccgctatggttacacccgggccgcccagatgatgggagagaagcagtctctacggccggctttgctgatgcttcagaagcagctctccctgccccagactggtgagctggacagccagacactaaaggccattcgaacaccacgctgtggtgtcccagacgtgggtcgattccaaaccttcaaaggcctcaagtgggaccatcataacatcacatactggatccaaaactactctgaagacttgccgcgagacatgatcgatgacgccttcgcgcgcgccttcgcggtgtggggcgaggtggcacccctcaccttcacccgcgtgtacggacccgaagcggacattgtcatccagtttggtgtcgcggagcacggagacgggtatcccttcgacggcaaggacggccttctggcacacgcctttccccctggcgccggcgttcagggagatgcccatttcgacgacgacgagttgtggtcgctgggcaaaggcgtcgtgatccccacttactatggaaactcaaatggtgccccatgtcactttcccttcaccttcgagggacgctcctattcggcctgcaccacagacggccgcaacgacggcacgccttggtgtagcacaacagctgactacgataaggacggcaaatttggtttctgccctagtgagagactctacacggagcacggcaacggagaaggcaaaccctgtgtgttcccgttcatctttgagggccgctcctactctgcctgcaccactaaaggccgctcggatggttaccgctggtgcgccaccacagccaactatgaccaggataaactgtatggcttctgccctacccgagtggacgcgaccgtagttgggggcaactcggcaggagagctgtgcgtcttccccttcgtcttcctgggcaagcagtactcttcctgtaccagcgacggccgcagggatgggcgcctctggtgtgcgaccacatcgaacttcgacactgacaagaagtggggtttctgtccagaccaagggtacagcctgttcctggtggcagcgcacgagttcggccatgcactgggcttagatcattccagcgtgccggaagcgctcatgtacccgctgtatagctacctcgagggcttccctctgaataaagacgacatagacggcatccagtatctgtatggtcgtggctctaagcctgacccaaggcctccagccaccaccacaactgaaccacagccgacagcacctcccactatgtgtcccactatacctcccacggcctatcccacagtgggccccacggttggccctacaggcgccccctcacctggccccacaagcagcccgtcacctggccctacaggcgccccctcacctggccctacagcgccccctactgcgggctcttctgaggcctctacagagtctttgagtccggcagacaatccttgcaatgtggatgtttttgatgctattgctgagatccagggcgctctgcatttcttcaaggacggttggtactggaagttcctgaatcatagaggaagcccattacagggccccttccttactgcccgcacgtggccagccctgcctgcaacgctggactccgcctttgaggatccgcagaccaagagggttttcttcttctctggacgtcaaatgtgggtgtacacaggcaagaccgtgctgggccccaggagtctggataagttgggtctaggcccagaggtaacccacgtcagcgggcttctcccgcgtcgtctcgggaaggctctgctgttcagcaaggggcgtgtctggagattcgacttgaagtctcagaaggtggatccccagagcgtcattcgcgtggataaggagttctctggtgtgccctggaactcacacgacatcttccagtaccaagacaaagcctatttctgccatggcaaattcttctggcgtgtgagtttccaaaatgaggtgaacaaggtggaccatgaggtgaaccaggtggacgacgtgggctacgtgacctacgacctcctgcagtgcccttgaactagggctccttctttgcttcaaccgtgcagtgcaagtctctagagaccaccaccaccaccaccacacacaaaccccatccgagggaaaggtgctagctggccaggtacagactggtgatctcttctagagactgggaaggagtggaggcaggcagggctctctctgcccaccgtcctttcttgttggactgtttctaataaacacggatccccaaccttttccagctactttagtcaatcagcttatctgtagttgcagatgcatccgagcaagaagacaactttgtagggtggattctgaccttttatttttgtgtggcgtctgagaattgaatcagctggcttttgtgacaggcacttcaccggctaaaccacctctcccgactccagcccttttatttattatgtatgaggttatgttcacatgcatgtatttaacccacagaatgcttactgtgtgtcgggcgcggctccaaccgctgcataaatattaaggtattcagttgcccctactggaaggtattatgtaactatttctctcttacattggagaacaccaccgagctatccactcatcaaacatttattgagagcatccctagggagccaggctctctactgggcgttagggacagaaatgttggttcttccttcaaggattgctcagagattctccgtgtcctgtaaatctgctgaaaccagaccccagactcctctctctcccgagagtccaactcactcactgtggttgctggcagctgcagcatgcgtatacagcatgtgtgctagagaggtagagggggtctgtgcgttatggttcaggtcagactgtgtcctccaggtgagatgacccctcagctggaactgatccaggaaggataaccaagtgtcttcctggcagtcttttttaaataaatgaataaatgaatatttacttaaaaaaaaaaaaaaaaaaaaaaaaaaaaaaaaaaaaaaaaaaaaaaa

SEQ ID NO: 16 - protein, 730 aa, Mus musculus
MSPWQPLLLALLAFGCSSAAPYQRQPTFVVFPKDLKTSNLTDTQLAEAYLYRYGYTRAAQMMGEKQSLRPALLMLQKQLSLPQTGELDSQTLKAIRTPRCGVPDVGRFQTFKGLKWDHHNITYWIQNYSEDLPRDMIDDAFARAFAVWGEVAPLTFTRVYGPEADIVIQFGVAEHGDGYPFDGKDGLLAHAFPPGAGVQGDAHFDDDELWSLGKGVVIPTYYGNSNGAPCHFPFTFEGRSYSACTTDGRNDGTPWCSTTADYDKDGKFGFCPSERLYTEHGNGEGKPCVFPFIFEGRSYSACTTKGRSDGYRWCATTANYDQDKLYGFCPTRVDATVVGGNSAGELCVFPFVFLGKQYSSCTSDGRRDGRLWCATTSNFDTDKKWGFCPDQGYSLFLVAAHEFGHALGLDHSSVPEALMYPLYSYLEGFPLNKDDIDGIQYLYGRGSKPDPRPPATTTTEPQPTAPPTMCPTIPPTAYPTVGPTVGPTGAPSPGPTSSPSPGPTGAPSPGPTAPPTAGSSEASTESLSPADNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRLGKALLFSKGRVWRFDLKSQKVDPQSVIRVDKEFSGVPWNSHDIFQYQDKAYFCHGKFFWRVSFQNEVNKVDHEVNQVDDVGYVTYDLLQCP

SEQ ID NO: 17 - DNA, 1721 bp, Mus musculus
ggcacgaagaaggatcggtttttaaagtaaagactgtctgtatggagccactagccatcctggcactgctgagcctaccaatctgctcagcgtatcctctgcatggggcagtgacacaaggccacccaagcatggatcttgctcagcaatacctagaaaaatactacaactttaaaaaaaatgagaaacaaattttcaaaagaaaggacagtagtcctgttgtcaaaaaaattcaagaaatgcagaagttcctcgggttggagatgacagggaagctggactccaacactatggagctgatgcacaagcccaggtgtggtgttcctgatgttggtggcttcagtaccttcccaggttcgccaaaatggaggaaatcccacatcacctacaggattgtgaattatacaccagatttgccaagacagagtgtggattctgccattgagaaagctttgaaggtctgggaggaggtgaccccactcactttctccaggatctctgaaggagaggctgacataatgatctcctttgcagttggagaacacggagacttttacccttttgatgggccaggacagagtctggctcatgcctacccacctggccctggattttatggagatgttcacttcgatgatgatgagaaatggacacttgcaccctcagggaccaacttattcctggttgcagcccatgaacttggccactccctgggtctctttcattccgacaagaaagaatctctgatgtacccagtctacaggttctccacaagcccagctaacttccacctttctcaagatgatatagagggcattcaatccctgtatggagccggtccctcctccgatgccacagtggttcctgtgttgtctgtctctccaagacctgagaccccagacaaatgtgatcctgctttgtcctttgattcagtcagcacgctgagaggggaagtcctattctttaaagacaggtacttctggcgcagatcccattggaatcccgagcctgaatttcatttgatatcagcattttggcccactcttccttcagacttagatgctgcctatgaggctcacaacacggacagtgttctgatttttaaaggaagtcagttctgggcagtccgaggaaatgaagtccaagcaggctacccaaaggggatccacactctcggttttcctcccaccgtgaagaagattgatgcagctgtttttgaaaaggagaagaagaaaacgtacttctttgtaggggacaaatactggagatttgatgagacaagacatgttatggataaaggcttcccaagacagataacagatgattttccaggaattgagccacaagttgatgctgtgttacacgaatttgggtttttttatttcttccgaggatcatcacagttcgagtttgaccccaatgccaggacggtgacacacatactgaagagcaacagctggctgctgtgctgatcatcatgacaagacatatacaaccctgtaaaatgagtctcatgacttcccacctactttattatgtgtcagaatgattagttgctcctgcatgttctgtggctccggatgagcgcagcagatgtctttcataatgagtcacaaagcatcacctgagcacagaagtgaaattttctcactgcagtaggtgagaggatgcatccatccccatgggtattttattatttaataaagagctttatttttgaa

SEQ ID NO: 18 - protein, 476 aa, Mus musculus
MEPLAILALLSLPICSAYPLHGAVTQGHPSMDLAQQYLEKYYNFKKNEKQIFKRKDSSPVVKKIQEMQKFLGLEMTGKLDSNTMELMHKPRCGVPDVGGFSTFPGSPKWRKSHITYRIVNYTPDLPRQSVDSAIEKALKVWEEVTPLTFSRISEGEADIMISFAVGEHGDFYPFDGPGQSLAHAYPPGPGFYGDVHFDDDEKWTLAPSGTNLFLVAAHELGHSLGLFHSDKKESLMYPVYRFSTSPANFHLSQDDIEGIQSLYGAGPSSDATVVPVLSVSPRPETPDKCDPALSFDSVSTLRGEVLFFKDRYFWRRSHWNPEPEFHLISAFWPTLPSDLDAAYEAHNTDSVLIFKGSQFWAVRGNEVQAGYPKGIHTLGFPPTVKKIDAAVFEKEKKKTYFFVGDKYWRFDETRHVMDKGFPRQITDDFPGIEPQVDAVLHEFGFFYFFRGSSQFEFDPNARTVTHILKSNSWLLC

SEQ ID NO: 19 - DNA, 2660 bp, Mus musculus
gctgggcaccatgcattcagctatcctggccaccttcttcttgttgagctggactccctgttggtccctgccccttccctatggtgatgatgatgatgatgacctgtctgaggaagaccttgtgtttgcagagcactacttgaaatcatactaccatcctgcgactcttgcgggaatcctgaagaagtctacagtgacctccacagttgacaggctccgagaaatgcaatctttctttggcttagaggtgactggcaaacttgatgatcccaccttagacatcatgagaaaaccaagatgtggagtgcctgatgtgggtgaatacaatgttttccctagaacactcaaatggtcccaaacgaacttaacttacaggattgtgaactatactcctgatatgtcccattctgaagtggagaaggccttcagaaaagccttcaaggtctggtctgatgtgacaccactgaatttcaccagaatctatgatggcactgctgacatcatgatatcttttgggactaaagaacatggtgacttctacccatttgatggaccttctggtcttctggcacacgcttttcctcctggaccaaactatggtggggatgcccattttgatgatgatgaaacctggacaagcagttccaaaggctacaacttgtttattgttgctgcccatgagcttggccactccctaggtctggatcactccaaggacccaggagccctgatgtttcccatctatacctacactggcaaaagccatttcatgcttcctgatgatgacgttcaaggaattcagtttctttatggtccaggcgatgaagaccccaaccctaagcatcccaaaacaccagagaagtgtgacccagccctatcccttgatgccattaccagtctccgaggagaaactatgatctttaaagacagattcttctggcgcctgcaccctcagcaggttgaggctgagctctttttgacaaagtccttttggccagaacttcccaaccatgtggatgctgcatatgaacatccatcccgtgaccttatgtttatctttagagggagaaaattctgggctctgaatggttatgacattctggaaggttatcccagaaaaatatctgacctgggattcccaaaagaggtgaagagactgagcgctgcggttcactttgagaacacggggaagaccctcttcttctctgagaaccacgtgtggagttatgatgatgttaaccagactatggacaaagattatccccgcctcatagaagaggaattccctggaattggcaacaaagtagatgctgtctatgagaaaaatggctatatctactttttcaatgggcccatacagtttgaatacagtatctggagtaatcgcattgtgagagtcatgccaacaaattccatattgtggtgttaagcatctttaaaagttgttatttatctcccagagagtatttggaatactttcagatgtatggggtgggggtggggtggagatatcaggggagagcttagttctgtgaacgagcttcagtaagttatctttgagcatacagtatctatatgactatgcgtggctggaaccacatggaagaattttaaagtaatgcaattgagaaccccaaggatcacctgattcttgcgtgctatgaagaaacaagattgataataacccacagcaaacatggggtccatctgcttttgagagcatgcataattattaatatatttattttaaaaagcctaacagacataaaataaatcatatttatataactgaattgtctttacaaaaaagtataaacttagaaacttgaaaattgtgaggagttcatgtatggggagccacagatgagcacagataaagggaaatgcctaaaaaatgcacgttaacggacaactttccaaagagagatttcagcttttcactgcgagcgttcagatttacatccacttttatacaaccaataaaaaaataccaaagtcactaaagaaaggggataacagccactacaaggacagtggaggtggccttacatttggcttaatttttatgttggtcattactcaaggctatgcacactggtagaagatattgagagagaaatggaggagatttctctttttattaaatatttaggcattgaaaagaccatagtgtgaaaagtcaaaattgctataagatacgtaagcaatgccatagctttttcatgaattatttgactattttagaataaaactaatgtttcaaccttgtttatctacccacttgttctaatgacctatagactctttgatacatagtctcttttctagtaacttgtgtgacaggggctaaggcagaaatattatgtagaagtagatccagctaagacacagcaagccagaataaagactgtgccagctggtcagtcgcccttttgagaccactcctttgtgctccaccatgtttgttaatccctctctgctttccttagcgagtaacacttggtgcttactgatgtgtgaaaagctattgtgtcaagagacagtgttaattaaactgggaaaatacaaaagaactgtttttttgaataatatgttagactgtatttatgttgtttctaataaaaataagtgttttcagcagaaaaaaaaaaaaaaaaaaa

SEQ ID NO: 20 - protein, 472 aa, Mus musculus
MHSAILATFFLLSWTPCWSLPLPYGDDDDDDLSEEDLVFAEHYLKSYYHPATLAGILKKSTVTSTVDRLREMQSFFGLEVTGKLDDPTLDIMRKPRCGVPDVGEYNVFPRTLKWSQTNLTYRIVNYTPDMSHSEVEKAFRKAFKVWSDVTPLNFTRIYDGTADIMISFGTKEHGDFYPFDGPSGLLAHAFPPGPNYGGDAHFDDDETWTSSSKGYNLFIVAAHELGHSLGLDHSKDPGALMFPIYTYTGKSHFMLPDDDVQGIQFLYGPGDEDPNPKHPKTPEKCDPALSLDAITSLRGETMIFKDRFFWRLHPQQVEAELFLTKSFWPELPNHVDAAYEHPSRDLMFIFRGRKFWALNGYDILEGYPRKISDLGFPKEVKRLSAAVHFENTGKTLFFSENHVWSYDDVNQTMDKDYPRLIEEEFPGIGNKVDAVYEKNGYIYFFNGPIQFEYSIWSNRIVRVMPTNSILWC

SEQ ID NO: 21 - DNA, 2597 bp, Mus musculus
ggagaagggagggaccaaaggagagcagagagggcttccaactcagttcgccgactaagcagaagaaagatcaaaaaacggaaaagagaagagcaaacagacatttccaggagcaattccctcacctccaagccgaccgcgctctaggaatccacattccgttcctttagaagacaaaggcgccccaagagaggcggcgcgaccccagggcgtgggccccgccgcggagcccgcaccgcccggcgccccgacgccggggaccatgtctcccgcccctcgaccctcccgcagcctcctgctccccctgctcacgcttggcacggcgctcgcctccctcggctgggcccaaggcagcaacttcagccccgaagcctggctgcagcagtatggctacctacctccaggggacctgcgtacccacacacaacgctcaccccagtcactctcagctgccattgccgccatgcaaaagttctatggtttacaagtgacaggcaaggctgatttggcaaccatgatggccatgaggcgccctcgctgtggtgttccggataagtttgggactgagatcaaggccaatgttcggaggaagcgctatgccattcagggcctcaagtggcagcataatgagatcactttctgcattcagaattacacccctaaggtgggcgagtatgccacattcgaggccattcggaaggccttccgagtatgggagagtgccacgccactgcgcttccgagaagtgccctatgcctacatccgggagggacatgagaagcaggctgacatcatgatcttgtttgctgagggtttccacggcgacagtacaccctttgatggtgaaggagggttcctggctcatgcctacttcccaggccccaatattggaggggatacccactttgattctgccgagccctggactgtccaaaatgaggatctaaatgggaatgacatcttcttggtggctgtgcatgagttggggcatgccctaggcctggaacattccaatgatccctccgccatcatggcccccttttaccagtggatggacacagagaacttcgtgttgcctgatgacgatcgccgtggcatccagcaactttatggaagcaagtcagggtcacccacaaagatgccccctcaacccagaactacctctcggccctctgtcccagataagcccaaaaaccccgcctatgggcccaacatctgtgacgggaactttgacaccgtggccatgctccgaggagagatgtttgtcttcaaggagcgatggttctggcgggtgaggaataaccaagtgatggatggatacccaatgcccattggccaattctggaggggcctgcctgcatccatcaatactgcctacgagaggaaggatggcaaatttgtcttcttcaaaggagataagcactgggtgtttgacgaagcctccctggaacccgggtaccccaagcacattaaggagctgggccgagggctgcccacggacaagatcgatgcagctctcttctggatgcccaatgggaagacctacttcttccggggcaataagtactaccggttcaatgaagaattcagggcagtggacagcgagtaccctaaaaacatcaaagtctgggaaggaatccctgaatctcccagggggtcattcatgggcagtgatgaagtcttcacatacttctacaagggaaacaaatactggaagttcaacaaccagaagctgaaggtagagccagggtatcccaagtcagctctgcgggactggatgggctgcccttcggggggccggcccgatgaggggactgaggaggagacggaggtgatcatcattgaggtggatgaggagggcagtggagctgtgagtgcggccgccgtggtcctgccggtactactgctgctcctggtactggcagtgggcctcgctgtcttcttcttcagacgccatgggacgcccaagcgactgctttactgccagcgttcgctgctggacaaggtctgacccccaccactggcccacccgcttctaccacaaggactttgcctctgaaggccagtggctacaggtggtagcaggtgggctgctctcacccgtcctgggctccctccctccagcctcccttctcagtccctaattggcctctcccaccctcaccccagcattgcttcatccataagtgggtcccttgagggctgagcagaagacggttggcctctggccctcaagggaatctcacagctcggtgtgtgttcagccctagttgaatgttgtcaaggctctgcacttgaaggcaagaccctctgaccttataggcaacggccaaatggggccatctgcttcttttccatccccctaactacataccttaaatctctgaactctgacctcaggaggctctgggcatatgagccctatatgtaccaagtgtacctagttggctgcctcccgccactctgactaaaaggaatcttaagagtgtacgtttggaggtggaaagattgttcagtttaccctaaagactttgataagaaagagaaagaaagaaagaaagaaagaaagaaagaaagaaagaaagaaagaaagaaag

SEQ ID NO: 22 - protein, 582 aa, Mus musculus
MSPAPRPSRSLLLPLLTLGTALASLGWAQGSNFSPEAWLQQYGYLPPGDLRTHTQRSPQSLSAAIAAMQKFYGLQVTGKADLATMMAMRRPRCGVPDKFGTEIKANVRRKRYAIQGLKWQHNEITFCIQNYTPKVGEYATFEAIRKAFRVWESATPLRFREVPYAYIREGHEKQADIMILFAEGFHGDSTPFDGEGGFLAHAYFPGPNIGGDTHFDSAEPWTVQNEDLNGNDIFLVAVHELGHALGLEHSNDPSAIMAPFYQWMDTENFVLPDDDRRGIQQLYGSKSGSPTKMPPQPRTTSRPSVPDKPKNPAYGPNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSGGRPDEGTEEETEVIIIEVDEEGSGAVSAAAVVLPVLLLLLVLAVGLAVFFFRRHGTPKRLLYCQRSLLDKV

SEQ ID NO: 23 - DNA, 3635 bp, Mus musculus
ccggcctgcactggccgccagccaccgagaggaggagcagaggatcctcggagcgcaataaaacggcggctcggcccgagcccgcagcaaacacagccatagaaggcagcggaggagccgagccgggctgcgctcgctcgccgccccccagcctctttcttctccgccgggtgcactgccctgcgccgtcccctcgccgctgcgccccttgacaaagaggacagaaagtttgcgcgggggagcgggccaggtgaggaggggcgtgcccggcgccccagtccgcgccccagcagccggacccaggcccccagcgcgcccgccatgggcgccgcggcccgcagcctccggctggcgctcggcctcctgctgctagccacgctgctgcgcccggccgacgcctgcagctgctccccggtgcacccgcaacaggcgttttgcaatgcagacgtagtgatcagagccaaagcagtgagcgagaaggaggtggattccgggaatgacatctatggcaaccccatcaagaggattcagtatgagatcaagcagataaagatgttcaaaggacctgacaaagacatcgagtttatctacacggccccctcttcagcagtgtgcggggtctcgctggacgttggaggaaagaaggagtatctaattgcaggaaaggcagaaggagatggcaagatgcacattaccctctgtgacttcattgtgccctgggacacgcttagcatcacccagaagaagagcctgaaccacaggtaccagatgggctgtgagtgcaagatcactcgctgtcccatgatcccttgctacatctcctccccggatgagtgcctctggatggactgggtcacagagaagagcatcaatgggcaccaggccaagttcttcgcctgcatcaagagaagtgatggttcttgcgcgtggtaccgcggggcggcaccccccaagcaagagtttcttgacatcgaggacccgtaagaaggctgacagagcccctgtggccaattgaaaagcctctgagggtttagactggtccagctttgacatcccttcctggaaacagcatgaataaaacatcaatcatccaagtgggttcacgctagtgtgattctgccccctcccctattttccctagacatggtagtgggtctggagggacaggcgggccaggttccctgccataccccttccctctgccagcctgagcactgtgtgtctcagtctttgatccttgctacaggcaggagtggagcacagacttgttaccaggtctctctggcactgtcacatgcagcagacaggcagcattaagggtaccctagctctgttagggcagagcctgggaatgtgcattttgcagaaactcttgaaggttgttgtaagactgtgtagccggcctaccaggtccttttcatcctgagagtgacatgtccctcgttttctgcagtggccacctctctctctggcccttgcaaatgcttcccatccctcctgcatctggtatggactttcaggaccctggtctccctcgggtctaagaatcaccctccaaccagtggttcatttttctaggagtcccagtcagccccatgaatccacagacttcagcgaatggaagccctccctgagccgtgtttctggcttcaaccaagtcattgctgccttcctctcccctgtctctacacacaccctcagtggggtctgtgaggtctcatgctgggggcagggatctgtggtgaggggtgcttggcagtccttgttgccactctcaagcttcccaagccattcttcacccctttccaagcaagcttcaagcatccaggctgagcagcacggctcggtttggctctctgtcgcatcaggccctgccgctgttggggggcgctaccagcactccctcttttgcacaaactgatgatataaaaggccagtcctaggcacctaggaaaagtctagtgaactctccctgctagatcagcggtcattatgaccctgttgatttctgtgtcagtaactaagcacgcagcaggggagactttgcggtgggcagttcttcgtaccaagcccccccccccaaggatgctgtggcttgttttcagatccatctcattttcctaaaggtgaattctcatgcatggctgagagacatgtgtatgcagctctgcttctgtctcttaatgtcgcttaaggcccttatagggaactggtatatctacttgctccttaaagcaattttctttctgatgccaataggatgggggttaagacaggcagagctttatcactaacaatatagacagccactcttccttctgcctgcgtcttaaaaaataagctgtcccaaggacacaaatgtatattatccatacatgcacgcatatgctcacacacaaactcagactctgaagtctggtagcctgtgaatgttccttttgtaaaatgcttccaaaagcctctttgctccaaccctgtcctaaccatcagaaaccccaaagaaacggttaaggactccccctcagactctccctacccaagcccctacaccaggacctggccagtccttttaagacagactgggaggacacacaggagtcagcctgccccttctgagggcattttcgtgttgtgcagtgatgttcttccttggatgctggcctggaccagccaacgagaccctgcagtctatcccgccctgcctgtttgcttcctgtgcggtggtatcaatatgtaacagtgcctgtttctgctgatttcatgacatgttctggtttgtttctgatgttcgccgtgagcgttcttgtgccgtgttgatgcctttcgtagcattagactttgcacttttaaaaaaaaaaaaaacaaaaatgttgaagcatcgaggaagcatcgaacccagagtggaatgcatggtatggtagctggcctgggacagagggaccctttctcatcttcctttgagttctttgactatctgcttttccagcctctcccgtcttttgtatctggttcaaattattataaaggaaagaccctctgagtataccggttctgaaagacggcctttctgttttccactcatgctggggtttctagccacaccaggcagatgagaggaaaccgagcgagcaaacgaacctttgggacaaagtgccagatggcagctgagcaacagccactcaaatgccttcccagcaaaccaattgcaatatatagtttaaggtgttgttttacttctgttatattctaagccctgggcctccctcccttactcccgtcatgccagcaactcgcaatatttcagatgacgtttacatggtagcaatttccaaatcgctgcctgatgcgtattaagacatatccgtgggcttgctgcataactcaacgctttgttgattttgtttctgtttgaactcttggttgtagggggggaggggtggaaccccatgtgcgtgctggaatatgaagtctgagatgtaccccccaacaccccacgctggcgatacgtgagagttgttgaaagtcagcaagccgagcgcgcctgatgctctgtatcagtctctacttttatttttatgagtttgctctgtcaatggacaataaaccatattatcaaagagaaaaaaaaaaaaaaa

SEQ ID NO: 24 - protein, 220 aa, Mus musculus
MGAAARSLRLALGLLLLATLLRPADACSCSPVHPQQAFCNADVVIRAKAVSEKEVDSGNDIYGNPIKRIQYEIKQIKMFKGPDKDIEFIYTAPSSAVCGVSLDVGGKKEYLIAGKAEGDGKMHITLCDFIVPWDTLSITQKKSLNHRYQMGCECKITRCPMIPCYISSPDECLWMDWVTEKSINGHQAKFFACIKRSDGSCAWYRGAAPPKQEFLDIEDP

SEQ ID NO: 25 - DNA, 27 bp, Artificial Sequence
  Description of Artificial Sequence Synthetic primer
catatcatgacagtgctggcgccagcc

SEQ ID NO: 26 - DNA, 28 bp, Artificial Sequence
  Description of Artificial Sequence Synthetic primer
gtaaggatcctagggatgggaggggagg

SEQ ID NO: 27 - DNA, 25 bp, Artificial Sequence
  Description of Artificial Sequence Synthetic primer
ggctctgaatggttatgacattctg

SEQ ID NO: 28 - DNA, 21 bp, Artificial Sequence
  Description of Artificial Sequence Synthetic primer
agagggtcttccccgtgttct

SEQ ID NO: 29 - DNA, 22 bp, Artificial Sequence
  Description of Artificial Sequence Synthetic primer
cgtgctgactgaatcaaaggac

SEQ ID NO: 30 - DNA, 24 bp, Artificial Sequence
  Description of Artificial Sequence Synthetic primer
cctgtgttgtctgtctctccaaga

SEQ ID NO: 31 - DNA, 21 bp, Artificial Sequence
  Description of Artificial Sequence Synthetic primer
ggaaactcacacgccagaaga

SEQ ID NO: 32 - DNA, 19 bp, Artificial Sequence
  Description of Artificial Sequence Synthetic primer
agcgtcattcgcgtggata

SEQ ID NO: 33 - DNA, 20 bp, Artificial Sequence
  Description of Artificial Sequence Synthetic primer
ctcagagtacgccagggaac

SEQ ID NO: 34 - DNA, 20 bp, Artificial Sequence
  Description of Artificial Sequence Synthetic primer
catggaaagcctctgtggat

SEQ ID NO: 35 - DNA, 20 bp, Artificial Sequence
  Description of Artificial Sequence Synthetic primer
ctcagagtacgccagggaac

SEQ ID NO: 36 - DNA, 20 bp, Artificial Sequence
  Description of Artificial Sequence Synthetic primer
gtccatccagaggcactcat

SEQ ID NO: 37 - DNA, 19 bp, Artificial Sequence
  Description of Artificial Sequence Synthetic primer
caatgggcattgggtatcc

SEQ ID NO: 38 - DNA, 19 bp, Artificial Sequence
  Description of Artificial Sequence Synthetic primer
aactttgacaccgtggcca

SEQ ID NO: 39 - DNA, 20 bp, Artificial Sequence
  Description of Artificial Sequence Synthetic primer
aaggaccggtttatttggcg

SEQ ID NO: 40 - DNA, 20 bp, Artificial Sequence
  Description of Artificial Sequence Synthetic primer
ctcccccgatgctgatactg

SEQ ID NO: 41 - DNA, 20 bp, Artificial Sequence
  Description of Artificial Sequence Synthetic primer
gatctcgctcctggaagatg

SEQ ID NO: 42 - DNA, 20 bp, Artificial Sequence
  Description of Artificial Sequence Synthetic primer
caatgaccccttcattgacc

SEQ ID NO: 43 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 44 - DNA, 931 bp, Homo sapiens
tttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagcagaaaaaaaaaaaaaaaa

SEQ ID NO: 45 - protein, 205 aa, Mus musculus
MMAPFASLASGILLLLSLIASSKACSCAPPHPQTAFCNSDLVIRAKFMGSPEINETTLYQRYKIKMTKMLKGFKAVGNAADIRYAYTPVMESLCGYAHKSQNRSEEFLITGRLRNGNLHISACSFLVPWRTLSPAQQRAFSKTYSAGCGVCTVFPCLSIPCKLESDTHCLWTDQVLVGSEDYQSRHFACLPRNPGLCTWRSLGAR

SEQ ID NO: 46 - DNA, 905 bp, Mus musculus
aggctttgactccagcggtgggtggatgagtaatgcgtccaggaagcctggaggcagtgatttccccgccaactccgcccttcgcatggacatttattctccactgtgcagcccctgccgccatcatcgcagatcggggctcctagagacacaccagagcagataccatgatggccccctttgcatctctggcatctggcatcctcttgttgctatcactgatagcttccagtaaggcctgtagctgtgccccaccccacccacagacagccttctgcaactcggacctggtcataagggctaaattcatgggttccccagaaatcaacgagaccaccttataccagcgttataagatcaagatgactaagatgctaaaaggattcaaggctgtgggaaatgccgcagatatccggtacgcctacaccccagtcatggaaagcctctgtggatatgcccacaagtcccagaaccgcagtgaagagtttctcatcacgggccgcctaaggaacggaaatttgcacatcagtgcctgcagcttcttggttccctggcgtactctgagccctgctcagcaaagagctttctcaaagacctatagtgctggctgtggggtgtgcacagtgtttccctgtttatctatcccttgcaaactggagagtgacactcactgtttgtggacggatcaggtcctcgtgggctctgaggactaccagagccgtcactttgcttgcctgccacggaatccaggcttgtgcacctggagatcccttggggcccgatgacctgaagccttcccccaggaaaaactgaagcctgaacactgtctacttttcctccatctttctttctcttagatggtgaaataaagaactatcagacagcagcaaaaaaaaaaaaaaaaa